(19) United States
(12) Patent Application Publication
(10) Pub. No.: US 2003/0236215 A1
(43) Pub. Date: Dec. 25, 2003

(54) Polynucleotide encoding a polypeptide having heparanase activity and expression of same in genetically modified cells

(75) Inventors: Iris Pecker, Rishon LeZion (IL); Israel Vlodavsky, Mevaseret Zion (IL); Elena Feinstein, Rehovot (IL)

Correspondence Address: G.E. EHRLICH (1995) LTD., SUITE 207, 2001 JEFFERSON DAVIS HIGHWAY, ARLINGTON, VA, 22202, US

(73) Assignees: Insight Strategy & Marketing Ltd.; Hadasit Medical Research Services and Development Ltd.

(21) Appl. No.: 10/456,573

(22) Filed: Jun. 9, 2003

Related U.S. Application Data
(63) Continuation-in-part of application No. 09/435,739, filed on Nov. 8, 1999.
Application No. 09/435,739 is a continuation of application No. 09/258,892, filed on Mar. 1, 1999, now abandoned.
Application No. 09/258,892 is a continuation-in-part of application No. PCT/US98/17954, filed on Aug. 31, 1998.

(51) Int. Cl. A61K 48/00; C07H 21/04

(52) U.S. Cl. 514/44; 536/23.2

(57) ABSTRACT

A polynucleotide (hpa) encoding a polypeptide having heparanase activity, vectors including same, genetically modified cells expressing heparanase, a recombinant protein having heparanase activity and antisense oligonucleotides and constructs for modulating heparanase expression.

This is a continuation in part of U.S. patent application Ser. No. 09/435,739, filed Nov. 8, 1999, which is a continuation of U.S. patent application Ser. No. 09/258,892, filed Mar. 1, 1999, which is a continuation-in-part of PCT/US98/17954, filed Aug. 31, 1998, which in turn claims priority from U.S. patent application Ser. No. 08/922,170, filed Sep. 2, 1997, now U.S. Pat. No. 5,968,822, issued Oct. 19, 1999, and U.S. patent application Ser. No. 09/109,386, filed on Jul. 2, 1998, all of which are hereby incorporated by reference as if fully set forth herein.

### FIELD OF THE INVENTION

The present invention relates to a polynucleotide, referred to hereinbelow as hpa, encoding a polypeptide having heparanase activity, vectors (nucleic acid constructs) including same and genetically modified cells expressing heparanase. The invention further relates to a recombinant protein having heparanase activity and to antisense oligonucleotides, constructs and ribozymes for down regulating heparanase activity. In addition, the invention relates to heparanase promoter sequences and their uses.

### BACKGROUND OF THE INVENTION

Heparan sulfate proteoglycans: Heparan sulfate proteoglycans (HSPG) are ubiquitous macromolecules associated with the cell surface and extra cellular matrix (ECM) of a wide range of cells of vertebrate and invertebrate tissues (1-4). The basic HSPG structure includes a protein core to which several linear heparan sulfate chains are covalently attached. These polysaccharide chains are typically composed of repeating hexuronic and D-glucosamine disaccharide units that are substituted to a varying extent with N- and O-linked sulfate moieties and N-linked acetyl groups (1-4). Studies on the involvement of ECM molecules in cell attachment, growth and differentiation revealed a central role of HSPG in embryonic morphogenesis, angiogenesis, neurite outgrowth and tissue repair (1-5). HSPG are prominent components of blood vessels (3). In large blood vessels they are concentrated mostly in the intima and inner media, whereas in capillaries they are found mainly in the subendothelial basement membrane where they support proliferating and migrating endothelial cells and stabilize the structure of the capillary wall. The ability of HSPG to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion. Cleavage of the heparan sulfate (HS) chains may therefore result in degradation of the subendothelial ECM and hence may play a decisive role in extravasation of blood-borne cells. HS catabolism is observed in inflammation, wound repair, diabetes, and cancer metastasis, suggesting that enzymes which degrade HS play important roles in pathologic processes. Heparanase activity has been described in activated immune system cells and highly metastatic cancer cells (6-8), but research has been handicapped by the lack of biologic tools to explore potential causative roles of heparanase in disease conditions.

Involvement of Heparanase in Tumor Cell Invasion and Metastasis: Circulating tumor cells arrested in the capillary beds of different organs must invade the endothelial cell lining and degrade its underlying basement membrane (BM) in order to invade into the extravascular tissue(s) where they establish metastasis (9, 10). Metastatic tumor cells often attach at or near the intercellular junctions between adjacent endothelial cells. Such attachment of the metastatic cells is followed by rupture of the junctions, retraction of the endothelial cell borders and migration through the breach in the endothelium toward the exposed underlying BM (9). Once located between endothelial cells and the BM, the invading cells must degrade the subendothelial glycoproteins and proteoglycans of the BM in order to migrate out of the vascular compartment. Several cellular enzymes (e.g., collagenase IV, plasminogen activator, cathepsin B, elastase, etc.) are thought to be involved in degradation of BM (10). Among these enzymes is an endo-β-D-glucuronidase (heparanase) that cleaves HS at specific intrachain sites (6, 8, 11). Expression of a HS degrading heparanase was found to correlate with the metastatic potential of mouse lymphoma (11), fibrosarcoma and melanoma (8) cells. Moreover, elevated levels of heparanase were detected in sera from metastatic tumor bearing animals and melanoma patients (8) and in tumor biopsies of cancer patients (12).

The control of cell proliferation and tumor progression by the local microenvironment, focusing on the interaction of cells with the extracellular matrix (ECM) produced by cultured corneal and vascular endothelial cells, was investigated previously by the present inventors. This cultured ECM closely resembles the subendothelium in vivo in its morphological appearance and molecular composition. It contains collagens (mostly type III and IV, with smaller amounts of types I and V), proteoglycans (mostly heparan sulfate- and dermatan sulfate-proteoglycans, with smaller amounts of chondroitin sulfate proteoglycans), laminin, fibronectin, entactin and elastin (13, 14). The ability of cells to degrade HS in the cultured ECM( was studied by allowing cells to interact with a metabolically sulfate labeled ECM, followed by gel filtration (Sepharose 6B) analysis of degradation products released into the culture medium (11). while intact HSPG are eluted next to the void volume of the column (Kav<0.2, Mr˜0.5×10⁶), labeled degradation fragments of HS side chains are eluted more toward the Vₜ of the column (0.5<kav<0.8, Mr=5-7×10³) (11).

The heparanase inhibitory effect of various non-anticoagulant species of heparin that might be of potential use in preventing extravasation of blood-borne cells was also investigated by the present inventors. Inhibition of heparanase was best achieved by heparin species containing 16 sugar units or more and having sulfate groups at both the N and O positions. While O-desulfation abolished the heparanase inhibiting effect of heparin, O-sulfated, N-acetylated heparin retained a high inhibitory activity, provided that the N-substituted molecules had a molecular size of about 4,000 daltons or more (7). Treatment of experimental animals with heparanase inhibitors (e.g., non-anticoagulant species of heparin) markedly reduced (>90%) the incidence of lung metastases induced by B16 melanoma, Lewis lung carcinoma and mammary adenocarcinoma cells (7, 8, 16). Heparin fractions with high and low affinity to anti-thrombin III exhibited a comparable high anti-metastatic activity, indicating that the heparanase inhibiting activity of heparin, rather than its anticoagulant activity, plays a role in the anti-metastatic properties of the polysaccharide (7).

Heparanase activity in the urine of cancer patients: In an attempt to further elucidate the involvement of heparanase in tumor progression and its relevance to human cancer, urine samples for heparanase activity were screened (16a). Heparanase activity was detected in the urine of some, but not all, cancer patients. High levels of heparanase activity were determined in the urine of patients with an aggressive metastatic disease and there was no detectable activity in the urine of healthy donors.

Heparanase activity was also found in the urine of 20% of normal and microalbuminuric insulin dependent diabetes mellitus (IDDM) patients, most likely due to diabetic nephropathy, the most important single disorder leading to renal failure in adults.

Possible involvement of heparanase in tumor angiogenesis: Fibroblast growth factors are a family of structurally related polypeptides characterized by high affinity to heparin (17). They are highly mitogenic for vascular endothelial cells and are among the most potent inducers of neovascularization (17, 18). Basic fibroblast growth factor (bFGF) has been extracted from the subendothelial ECM produced in vitro (19) and from basement membranes of the cornea (20), suggesting that ECM may serve as a reservoir for bFGF. Immunohistochemical staining revealed the localization of bFGF in basement membranes of diverse tissues and blood vessels (21). Despite the ubiquitous presence of bFGF in normal tissues, endothelial cell proliferation in these tissues is usually very low, suggesting that bFGF is somehow sequestered from its site of action. Studies on the interaction of bFGF with ECM revealed that bFGF binds to HSPG in the ECM and can be released in an active form by HS degrading enzymes (15, 20, 22). It was demonstrated that heparanase activity expressed by platelets, mast cells, neutrophils, and lymphoma cells is involved in release of active bFGF from ECM and basement membranes (23), suggesting that heparanase activity may not only function in cell migration and invasion, but may also elicit an indirect neovascular response. These results suggest that the ECM HSPG provides a natural storage depot for bFGF and possibly other heparin-binding growth promoting factors (24, 25). Displacement of bFGF from its storage within basement membranes and ECM may therefore provide a novel mechanism for induction of neovascularization in normal and pathological situations.

Recent studies indicate that heparin and HS are involved in binding of bFGF to high affinity cell surface receptors and in bFGF cell signaling (26, 27). Moreover, the size of HS required for optimal effect was similar to that of HS fragments released by heparanase (28). Similar results were obtained with vascular endothelial cells growth factor (VEGF) (29), suggesting the operation of a dual receptor mechanism involving HS in cell interaction with heparin-binding growth factors. It is therefore proposed that restriction of endothelial cell growth factors in ECM prevents their systemic action on the vascular endothelium, thus maintaining a very low rate of endothelial cells turnover and vessel growth. On the other hand, release of bFGF from storage in ECM as a complex with HS fragment, may elicit localized endothelial cell proliferation and neovascularization in processes such as wound healing, inflammation and tumor development (24, 25).

Expression of heparanase by cells of the immune system: Heparanase activity correlates with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of HS by a specific heparanase activity (6). The enzyme is released from intracellular compartments (e.g., lysosomes, specific granules, etc.) in response to various activation signals (e.g., thrombin, calcium ionophore, immune complexes, antigens, mitogens, etc.), suggesting its regulated involvement in inflammation and cellular immunity.

Some of the observations regarding the heparanase enzyme were reviewed in reference No. 6 and are listed hereinbelow:

First, a proteolytic activity (plasminogen activator) and heparanase participate synergistically in sequential degradation of the ECM HSPG by inflammatory leukocytes and malignant cells.

Second, a large proportion of the platelet heparanase exists in a latent form, probably as a complex with chondroitin sulfate. The latent enzyme is activated by tumor cell-derived factor(s) and may then facilitate cell invasion through the vascular endothelium in the process of tumor metastasis.

Third, release of the platelet heparanase from α-granules is induced by a strong stimulant (i.e., thrombin), but not in response to platelet activation on ECM.

Fourth, the neutrophil heparanase is preferentially and readily released in response to a threshold activation and upon incubation of the cells on ECM.

Fifth, contact of neutrophils with ECM inhibited release of noxious enzymes (proteases, lysozyme) and oxygen radicals, but not of enzymes (heparanase, gelatinase) which may enable diapedesis. This protective role of the subendothelial ECM was observed when the cells were stimulated with soluble factors but not with phagocytosable stimulants.

Sixth, intracellular heparanase is secreted within minutes after exposure of T cell. lines to specific antigens.

Seventh, mitogens (Con A, LPS) induce synthesis and secretion of heparanase by normal T and B lymphocytes maintained in vitro. T lymphocyte heparanase is also induced by immunization with antigen in vivo.

Eighth, heparanase activity is expressed by pre-B lymphomas and B-lymphomas, but not by plasmacytomas and resting normal B lymphocytes.

Ninth, heparanase activity is expressed by activated macrophages during incubation with ECM, but there was little or no release of the enzyme into the incubation medium. Similar results were obtained with human myeloid leukemia cells induced to differentiate to mature macrophages.

Tenth, T-cell mediated delayed type hypersensitivity and experimental autoimmunity are suppressed by low doses of heparanase inhibiting non-anticoagulant species of heparin (30).

Eleventh, heparanase activity expressed by platelets, neutrophils and metastatic tumor cells releases active bFGF from ECM and basement membranes. Release of bFGF from storage in ECM may elicit a localized neovascular response in processes such as wound healing, inflammation and tumor development.

Twelfth, among the breakdown products of the ECM generated by heparanase is a tri-sulfated disaccharide that can inhibit T-cell mediated inflammation in vivo (31). This inhibition was associated with an inhibitory effect of the disaccharide on the production of biologically active TNFα by activated T cells in vitro (31).

Other potential therapeutic applications: Apart from its involvement in tumor cell metastasis, inflammation and autoimmunity, mammalian heparanase may be applied to modulate: bioavailability of heparin-binding growth factors (15); cellular responses to heparin-binding growth factors (e.g., bFGF, VEGF) and cytokines (IL-8) (31a, 29); cell interaction with plasma lipoproteins (32); cellular susceptibility to certain viral and some bacterial and protozoa infections (33, 33a, 33b); and disintegration of amyloid plaques (34). Heparanase may thus prove useful for conditions such as wound healing, angiogenesis, restenosis, atherosclerosis, inflammation, neurodegenerative diseases and viral infections. Mammalian heparanase can be used to neutralize plasma heparin, as a potential replacement of protamine. Anti-heparanase antibodies may be applied for immunodetection and diagnosis of micrometastases, autoimmune lesions and renal failure in biopsy specimens, plasma samples, and body fluids. Common use in basic research is expected.

The identification of the hpa gene encoding for heparanase enzyme will enable the production of a recombinant enzyme in heterologous expression systems. Availability of the recombinant protein will pave the way for solving the protein structure function relationship and will provide a tool for developing new inhibitors.

Viral Infection: The presence of heparan sulfate on cell surfaces have been shown to be the principal requirement for the binding of Herpes Simplex (33) and Dengue (33a) viruses to cells and for subsequent infection of the cells. Removal of the cell surface heparan sulfate by heparanase may therefore abolish virus infection. In fact, treatment of cells with bacterial heparitinase (degrading heparan sulfate) or heparinase (degrading heparan) reduced the binding of two related animal herpes viruses to cells and rendered the cells at least partially resistant to virus infection (33). There are some indications that the cell surface heparan sulfate is also involved in HIV infection (33b).

Neurodegenerative diseases: Heparan sulfate proteoglycans were identified in the prion protein amyloid plaques of Genstmann-Straussler Syndrome, Creutzfeldt-Jakob disease and Scrape (34). Heparanase may disintegrate these amyloid plaques which are also thought to play a role in the pathogenesis of Alzheimer's disease.

Restenosis and Atherosclerosis: Proliferation of arterial smooth muscle cells (SMCs) in response to endothelial injury and accumulation of cholesterol rich lipoproteins are basic events in the pathogenesis of atherosclerosis and restenosis (35). Apart from its involvement in SMC proliferation (i.e., low affinity receptors for heparin-binding growth factors), HS is also involved in lipoprotein binding, retention and uptake (36). It was demonstrated that HSPG and lipoprotein lipase participate in a novel catabolic pathway that may allow substantial cellular and interstitial accumulation of cholesterol rich lipoproteins (32). The latter pathway is expected to be highly atherogenic by promoting accumulation of apoB and apoE rich lipoproteins (i.e. LDL, VLDL, chylomicrons), independent of feed back inhibition by the cellular sterol content. Removal of SMC HS by heparanase is therefore expected to inhibit both SMC proliferation and lipid accumulation and thus may halt the progression of restenosis and atherosclerosis.

Gene Therapy:

The ultimate goal in the management of inherited as well as acquired diseases is a rational therapy with the aim to eliminate the underlying biochemical defects associated with the disease rather then symptomatic treatment. Gene therapy is a promising candidate to meet these objectives. Initially it was developed for treatment of genetic disorders, however, the consensus view today is that it offers the prospect of providing therapy for a variety of acquired diseases, including cancer, viral infections, vascular diseases and neurodegenerative disorders.

The gene-based therapeutic can act either intracellularly, affecting only the cells to which it is delivered, or extracellularly, using the recipient cells as local endogenous factories for the therapeutic product(s). The application of gene therapy may follow any of the following strategies: (i) prophylactic gene therapy, such. as using gene transfer to protect cells against viral infection; (ii) cytotoxic gene therapy, such as cancer therapy, where genes encode cytotoxic products to render the target cells vulnerable to attack by the normal immune response; (iii) biochemical correction, primarily for the treatment of single gene defects, where a normal copy of the gene is added to the affected or other cells.

To allow efficient transfer of the therapeutic genes, a variety of gene delivery techniques have been developed based on viral and non-viral vector systems. The most widely used and most efficient systems for delivering genetic material into target cells are viral vectors. So far, 329 clinical studies (phase I, I/II and II) with over 2,500 patients have been initiated Worldwide since 1989 (50).

The approach of gene addition pose serious barriers. The expression of many genes is tightly regulated and context dependent, so achieving the correct balance and function of expression is challenging. The gene itself is often quite large, containing many exons and introns. The delivery vector is usually a virus, which can infect with a high efficiency but may, on the other hand, induce immunological response and consequently decreases effectiveness, especially upon secondary administration. Most of the current expression vector-based gene therapy protocols fail to achieve clinically significant transgene expression required for treating genetic diseases. Apparently, it is difficult to deliver enough virus to the right cell type to elicit an effective and therapeutic effect (51)

Homologous recombination, which was initially considered to be of limited use for gene therapy because of its low frequency in mammalian cells, has recently emerged as a potential strategy for developing gene therapy. Different approaches have been used to study homologous recombination in mammalian cells; some involve DNA repair mechanisms. These studies aimed at either gene disruption or gene correction and include RNA/DNA chimeric oligonucleotides, small or large homologous DNA fragments, or adeno-associated viral vectors. Most of these studies show a reasonable frequency of homologous recombination, which warrants further in vivo testing (52). Homologous recombination-based gene therapy has the potential to develop into a powerful therapeutic modality for genetic diseases. It can offer permanent expression and normal regulation of corrected genes in appropriate cells or organs and probably can be used for treating dominantly inherited diseases such as polycystic kidney disease.

Genomic Sequences Function in Regulation of Gene Expression:

The efficient expression of therapeutic genes in target cells or tissues is an important component of efficient and safe gene therapy. The expression of genes is driven by the promoter region upstream of the coding sequence, although regulation of expression may be supplemented by farther upstream or downstream DNA sequences or DNA in the introns of the gene. Since this important information is embedded in the DNA, the description of gene structure is crucial to the analysis of gene regulation. Characterization of cell specific or tissue specific promoters, as well as other tissue specific regulatory elements enables the use of such sequences to direct efficient cell specific, or developmental stage specific gene expression. This information provides the basis for targeting individual genes and for control of their expression by exogenous agents, such as drugs. Identification of transcription factors and other regulatory proteins required for proper gene expression will point at new potential targets for modulating gene expression, when so desired or required.

Efficient expression of many mammalian genes depends on the presence of at least one intron. The expression of mouse thymidylate synthase (TS) gene, for example, is greatly influenced by intron sequences. The addition of almost any of the introns from the mouse TS gene to an intronless TS minigene leads to a large increase in expression (42). The involvement of intron 1 in the regulation of expression was demonstrated for many other genes. In human factor IX (hFIX), intron 1 is able to increase the expression level about 3 fold mare as compared to that of the hFIX cDNA (43). The expression enhancing activity of intron 1 is due to efficient functional splicing sequences, present in. the precursor mRNA. By being efficiently assembled into spliceosome complexes, transcripts with splicing sequences may be better protected in the nucleus from random degradations, than those without such sequences (44).

A forward-inserted intron1-carrying HFIX expression cassette suggested to be useful for directed gene transfer, while for retroviral-mediated gene transfer system, reversely-inserted intron 1-carrying HFIX expression cassette was considered (43).

A highly conserved cis-acting sequence element was identified in the first intron of the mouse and rat c-Ha-ras, and in the first exon of Ha- and Ki-ras genes of human, mouse and rat. This cis-acting regulatory sequence confers strong transcription enhancer activity that is differentially modulated by steroid hormones in metastatic and nonmetastatic subpopulations. Perturbations in the regulatory activities of such cis-acting sequences may play an important role in governing oncogenic potency of Ha-ras through transcriptional control mechanisms (45).

Intron sequences affect tissue specific, as well as inducible gene expression. A 182 bp intron 1 DNA segment of the mouse Col2a1 gene contains the necessary information to confer high-level, temporally correct, chondrocyte expression on a reporter gene in intact mouse embryos, while Col2a1 promoter sequences are dispensable for chondrocyte expression (46). In Col1A1 gene the intron plays little or no role in constitutive expression of collagen in the skin, and in cultured cells derived from the skin, however, in the lungs of young mice, intron deletion results in decrease of expression to less than 50% (47).

A classical enhancer activity was shown in the 2 kb intron fragment in bovine beta-casein gene. The enhancer activity was largely dependent on the lactogenic hormones, especially prolactin. It was suggested that several elements in the intron-1 of the bovine beta-casein gene cooperatively interact not only with each other but also with its promoter for hormonal induction (48).

Identification and characterization of regulatory elements in genomic non-coding sequences, such as introns, provides a tool for designing and constructing novel vectors for tissue specific, hormone regulated or any other defined expression pattern, for gene therapy. Such an expression cassette was developed, utilizing regulatory elements from the human cytokeratin 18 (K18) gene, including 5′ genomic sequences and one of its introns. This cassette efficiently expresses reporter genes, as well as the human cystic fibrosis transmembrane conductance regulator (CFTR) gene, in cultured lung epithelial cells (49).

Alternative Splicing:

Alternative splicing of pre mRNA is a powerful and versatile regulatory mechanism that can effect quantitative control of gene expression and functional diversification of proteins. It contributes to major developmental decisions and also to a fine-tuning of gene function. Genetic and biochemical approaches have identified cis-acting regulatory elements and trans-acting factors that control alternative splicing of specific mRNAs. This mechanism results in the generation of variant isoforms of various proteins from a single gene. These include cell surface molecules such as CD44, receptors, cytokines such as VEGF and enzymes. Products of alternatively spliced transcripts differ in their expression pattern, substrate specificity and other biological parameters.

The FGF receptor RNA undergoes alternative splicing which results in the production of several isoforms, which exhibit different ligand binding specificities. The alternative splicing is regulated in a cell specific manner (53).

Alternative spliced mRNAs are often correlated with malignancy. An increase in specific splice variant of tyrosinase was identified in murine melanomas (54). Multiple splicing variants of estrogen receptor are present in individual human breast tumors. CD44 has various isoform, some are characteristic of malignant tissues.

Identification of tumor specific alternative splice variants provide new tool for cancer diagnostics. CD44 variants have been used for detection of malignancy in urine samples from patients with urothelial cancer by competitive RT-PCR (55). CD44 exon 6 was suggested as prognostic indicator of metastasis in breast cancer (56).

Different enzymes or polypeptides generated by alternative splicing may have different function or catalytic specificity. The identification and characterization of the enzyme forms, which are involved in pathological processes, is crucial for the design of appropriate and efficient drugs.

Modulation of Gene Expression—Antisense Technology:

An antisense oligonucleotide (e.g., antisense oligodeoxyribonucleotide) may bind its target nucleic acid either by Watson-Crick base pairing or Hoogsteen and anti-Hoogsteen base pairing (64). According to the Watson-Crick base pairing, heterocyclic bases of the antisense oligonucleotide form hydrogen bonds with the heterocyclic bases of target single-stranded nucleic acids (RNA or single-stranded DNA), whereas according to the Hoogsteen base pairing, the heterocyclic bases of the target nucleic acid are double-stranded DNA, wherein a third strand is accommodated in the major groove of the B-form DNA duplex by Hoogsteen and anti-Hoogsteen base pairing to form a triple helix structure.

According to both the Watson-Crick and the Hoogsteen base pairing models, antisense oligonucleotides have the potential to regulate gene expression and to disrupt the essential functions of the nucleic acids in cells. Therefore, antisense oligonucleotides have possible uses in modulating a wide range of diseases in which gene expression is altered.

Since the development of effective methods for chemically synthesizing oligonucleotides, these molecules have been extensively used in biochemistry and biological research and have the potential use in medicine, since carefully devised oligonucleotides can be used to control gene expression by regulating levels of transcription, transcripts and/or translation.

Oligodeoxyribonucleotides as long as 100 base pairs (bp) are routinely synthesized by solid phase methods using commercially available, fully automated synthesis machines. The chemical synthesis of oligoribonucleotides, however, is far less routine. Oligoribonucleotides are also much less stable than oligodeoxyribonucleotides, a fact which has contributed to the more prevalent use of oligodeoxyribonucleotides in medical and biological research, directed at, for example, the regulation of transcription or translation levels.

Gene expression involves few distinct and well regulated steps. The first major step of gene expression involves transcription of a messenger RNA (mRNA) which is an RNA sequence complementary to the antisense (i.e., −) DNA strand, or, in other words, identical in sequence to the DNA sense (i.e., +) strand, composing the gene. In eukaryotes, transcription occurs in the cell nucleus.

The second major step of gene expression involves translation of a protein (e.g., enzymes, structural proteins, secreted proteins, gene expression factors, etc.) in which the mRNA interacts with ribosomal RNA complexes (ribosomes) and amino acid activated transfer RNAs (tRNAs) to direct the synthesis of the protein coded for by the mRNA sequence.

Initiation of transcription requires specific recognition of a promoter DNA sequence located upstream to the coding sequence of a gene by an RNA-synthesizing enzyme—RNA polymerase. This recognition is preceded by sequence-specific binding of one or more transcription factors to the promoter sequence. Additional proteins which bind at or close to the promoter sequence may trans upregulate transcription via cis elements known as enhancer sequences. Other proteins which bind to or close to the promoter, but whose binding prohibits the action of RNA polymerase, are known as repressors.

There are also evidence that in some cases gene expression is downregulated by endogenous antisense RNA repressors that bind a complementary mRNA transcript and thereby prevent its translation into a functional protein.

Thus, gene expression is typically upregulated by transcription factors and enhancers and downregulated by repressors.

However, in many disease situation gene expression is impaired. In many cases, such as different types of cancer, for various reasons the expression of a specific endogenous or exogenous (e.g., of a pathogen such as a virus) gene is upregulated. Furthermore, in infectious diseases caused by pathogens such as parasites, bacteria or viruses, the disease progression depends on expression of the pathogen genes, this phenomenon may also be considered as far as the patient is concerned as upregulation of exogenous genes.

Most conventional drugs function by interaction with and modulation of one or more targeted endogenous or exogenous proteins, e.g., enzymes. Such drugs, however, typically are not specific for targeted proteins but interact with other proteins as well. Thus, a relatively large dose of drug must be used to effectively modulate a targeted protein.

Typical daily doses of drugs are from 10⁻⁵-10⁻¹ millimoles per kilogram of body weight or 10⁻³-10 millimoles for a 100 kilogram person. If this modulation instead could be effected by interaction with and inactivation of. mRNA, a dramatic reduction in the necessary amount of drug could likely be achieved, along with a corresponding reduction in side effects. Further reductions could be effected if such interaction could be rendered site-specific. Given that a functioning gene continually produces mRNA, it would thus be even more advantageous if gene transcription could be arrested in its entirety.

Given these facts, it would be advantageous if gene expression could be arrested or downmodulated at the transcription level.

The ability of chemically synthesizing oligonucleotides and analogs thereof having a selected predetermined sequence offers means for downmodulating gene expression. Three types of gene expression modulation strategies may be considered.

At the transcription level, antisense or sense oligonucleotides or analogs that bind to the genomic DNA by strand displacement or the formation of a triple helix, may prevent transcription (64).

At the transcript level, antisense oligonucleotides or analogs that bind target mRNA molecules lead to the enzymatic cleavage of the hybrid by intracellular RNase H (65). In this case, by hybridizing to the targeted mRNA, the oligonucleotides or oligonucleotide analogs provide a duplex hybrid recognized and destroyed by the RNase H enzyme. Alternatively, such hybrid formation may lead to interference with correct splicing (66). As a result, in both cases, the number of the target mRNA intact transcripts ready for translation is reduced or eliminated.

At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs (67).

Thus, antisense sequences, which as described hereinabove may arrest the expression of any endogenous and/or exogenous gene depending on their specific sequence, attracted much attention by scientists and pharmacologists who were devoted at developing the antisense approach into a new pharmacological tool (68).

For example, several antisense oligonucleotides have been shown to arrest hematopoietic cell proliferation (69), growth (70), entry into the S phase of the cell cycle (71), reduced survival (72) and prevent receptor mediated responses (73). For use of antisense oligonucleotides as antiviral agents the reader is referred to reference 74.

For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity.

Unmodified oligonucleotides are impractical for use as antisense sequences since they have short in vivo half-lives, during which they are degraded rapidly by nucleases. Furthermore, they are difficult to prepare in more than milligram quantities. In addition, such oligonucleotides are poor cell membrane penetraters (75).

Thus it is apparent that in order to meet all the above listed requirements, oligonucleotide analogs need to be devised in a suitable manner. Therefore, an extensive search for modified oligonucleotides has been initiated.

For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done, nevertheless with little success.

Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, α-anomeric bridges and borane derivatives. For further details the reader is referred to reference 76.

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—). However, the application provides no data supporting the specific binding of an oligonucleotide analog to a target oligonucleotide.

International patent application WO 92/20702 describe an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other (77). PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal.

Thus, antisense technology requires pairing of messenger RNA with an oligonucleotide to form a double helix that inhibits translation. The concept of antisense-mediated gene therapy was already introduced in 1978 for cancer therapy. This approach was based on certain genes that are crucial in cell division and growth of cancer cells. Synthetic fragments of genetic substance DNA can achieve this goal. Such molecules bind to the targeted gene molecules in RNA of tumor cells, thereby inhibiting the translation of the genes and resulting in dysfunctional growth of these cells. Other mechanisms has also been proposed. These strategies have been used, with some success in treatment of cancers, as well as other illnesses, including viral and other infectious diseases. Antisense oligonucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used modification in antisense oligonucleotide ongoing clinical trials (57). A new generation of antisense molecules consist of hybrid antisense oligonucleotide with a central portion of synthetic DNA while four bases on each end have been modified with 2′O-methyl ribose to resemble RNA. In preclinical studies in laboratory animals, such compounds have demonstrated greater stability to metabolism in body tissues and an improved safety profile when compared with the first-generation unmodified phosphorothioate (Hybridon Inc. news). Dosens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived protein (57).

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy. A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

Another approach is the use of specific nucleic acid sequences to act as decoys for transcription factors. Since transcription factors bind specific DNA sequences it is possible to synthesize oligonucleotides that will effectively compete with the native DNA sequences for available transcription factors in vivo. This approach requires the identification of gene specific transcription factor (57).

Indirect inhibition of gene expression was demonstrated for matrix metalloproteinase genes (MMP-1, −3, and −9), which are associated with invasive potential of human cancer cells. E1AF is a transcription activator of MMP genes. Expression of E1AF antisense RNA in HSC3AS cells showed decrease in mRNA and protein levels of MMP-1, −3, and −9. Moreover, HSC3AS showed lower invasive potential in vitro and in vivo. These results imply that transfection of antisense inhibits tumor invasion by down-regulating MMP genes (58).

Ribozymes:

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials (62). More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Gene Disruption in Animal Models:

The emergence of gene inactivation by homologous recombination methodology in embryonic stem cells has revolutionized the field of mouse genetics. The availability of a rapidly growing number of mouse null mutants has represented an invaluable source of knowledge on mammalian development, cellular biology and physiology, and has provided many models for human inherited diseases. Animal models are required for an effective drug delivery development program and evaluation of gene therapy approach. The improvement of the original knockout strategy, as well as exploitation of exogenous enzymatic systems that are active in the recombination process, has been considerably extended the range of genetic manipulations that can be produced. Additional methods have been developed to provide versatile research tools: Double replacement method, sequential gene targeting, conditional cell type specific gene targeting, single copy integration method, inducible gene targeting, gene disruption by viral delivery, replacing one gene with another, the so called knock-in method and the induction of specific balanced chromosomal translocation. It is now possible to introduce a point mutation as a unique change in the entire genome, therefore allowing very fine dissection of gene function in vivo. Furthermore, the advent of methods allowing conditional gene targeting opens the way for analysis of consequence of a particular mutation in a defined organ and at a specific time during the life of the experimental animal (59).

DNA Vaccination:

Observations in the early 1990s that plasmid DNA could directly transfect animal cells in vivo sparked exploration of the use of DNA plasmids to induce immune response by direct injection into animal of DNA encoding antigenic protein. When a DNA vaccine plasmid enters the eukaryotic cell, the protein it encodes is transcribed and translated within the cell. In the case of pathogens, these proteins are presented to the immune system in their native form, mimicking the presentation of antigens during a natural infection. DNA vaccination is particularly useful for the induction of T cell activation. It was applied for viral and bacterial infectious diseases, as well as for allergy and for cancer. The central hypothesis behind active specific immunotherapy for cancer is that tumor cells express unique antigens that should stimulate the immune system. The first DNA vaccine against tumor was carcino-embrionic antigen (CEA). DNA vaccinated animals expressed immunoprotection and immunotherapy of human CEA-expressing syngeneic mouse colon and breast carcinoma (61). In a mouse model of neuroblastoma, DNA immunization with HuD resulted in tumor growth inhibition with no neurological disease (60). Immunity to the brown locus protein, gp⁷⁵ tyrosinase-related protein-1, associated with melanoma, was investigated in a syngeneic mouse model. Priming with human gp75 DNA broke tolerance to mouse gp75. Immunity against mouse gp75 provided significant tumor protection (60).

Glycosyl Hydrolases:

Glycosyl hydrolases are a widespread group of enzymes that hydrolyze the o-glycosidic bond between two or more carbohydrates or between a carbohydrate and a noncarbohydrate moiety. The enzymatic hydrolysis of glycosidic bond occurs by using major one or two mechanisms leading to overall retention or inversion of the anomeric configuration. In both mechanisms catalysis involves two residues: a proton donor and a nucleophile. Glycosyl hydrolyses have been classified into 58 families based on amino acid similarities. The glycosyl hydrolyses from families 1, 2, 5, 10, 17, 30, 35, 39 and 42 act on a large variety of substrates, however, they all hydrolyze the glycosidic bond in a general acid catalysis mechanism, with retention of the anomeric configuration. The mechanism involves two glutamic acid residues, which are the proton donors and the nucleophile, with an aspargine always preceding the proton donor. Analyses of a set of known 3D structures from this group revealed that their catalytic domains, despite the low level of sequence identity, adopt a similar (α/β) 8 fold with the proton donor and the nucleophile located at the C-terminal ends of strands β4 and β7, respectively. Mutations in the functional conserved amino acids of lysosomal glycosyl hydrolases were identified in lysosomal storage diseases. Lysosomal glycosyl hydrolases including β-glucuronidase, β-manosidase, β-glucocerebrosidase, β-galactosidase and α-L iduronidase, are all exo-glycosyl hydrolases, belong to the GH-A clan and share a similar catalytic site. However, many endo-glucanases from various organisms, such as bacterial and fungal xylenases and cellulases share this catalytic domain.

Genomic Sequence of hpa Gene and its Implications:

It is well established that heparanase activity is correlated with cancer metastasis. This correlation was demonstrated at the level of enzymatic activity as well as the levels of protein and hpa cDNA expression in highly metastatic cancer cells as compared with non-metastatic cells. As such, inhibition of heparanase activity is desirable, and has been attempted by several means. The genomic region, encoding the hpa gene and the surrounding, provides a new powerful tool for regulation of heparanase activity at the level of gene expression. Regulatory sequences may reside in noncoding regions both upstream)and downstream the transcribed region as well as in intron sequences. A DNA sequence upstream of the transcription start site contains the promoter region and potential regulatory elements. Regulatory factors, which interact with the promoter region may be identified and be used as potential drugs for inhibition of cancer, metastasis and inflammation. The promoter region can be used to screen for inhibitors of heparanase gene expression. Furthermore, the hpa promoter can be used to direct cell specific, particularly cancer cell specific, expression of foreign genes, such as cytotoxic or apoptotic genes, in order to specifically destroy cancer cells.

Cancer and yet unknown related genetic disorders may involve rearrangements and mutations in the heparanase gene, either in coding or non-coding regions. Such mutations may affect expression level or enzymatic activity. The genomic sequence of hpa enables the amplification of specific genomic DNA fragments, identification and diagnosis of mutations.

### SUMMARY OF THE INVENTION

There is thus a widely recognized need for, and it would be highly advantageous to have genomic, cDNA and composite polynucleotides encoding a polypeptide having heparanase activity, vectors including same, genetically modified cells expressing heparanase and a recombinant protein having heparanase activity, as well as antisense oligonucleotides, constructs and ribozymes which can be used for down regulation heparanase activity. Cloning of the human hpa gene which encodes heparanase, and expression of recombinant heparanase by transfected host cells is reported herein, as well as downregulation of heparanase activity by antisense technology.

A purified preparation of heparanase isolated from human hepatoma cells was subjected to tryptic digestion and microsequencing. The YGPDVGQPR (SEQ ID NO:8) sequence revealed was used to screen EST databases for homology to the corresponding back translated DNA sequence. Two closely related EST sequences were identified and were thereafter found to be identical. Both clones contained an insert of 1020 bp which included an open reading frame of 973 bp followed by a 27 bp of 3′ untranslated region and a Poly A tail. Translation start site was not identified.

Cloning of the missing 5′ end of hpa was performed by PCR amplification of DNA from placenta Marathon RACE cDNA composite using primers selected according to the EST clones sequence and the linkers of the composite. A 900 bp PCR fragment, partially overlapping with the identified 3′ encoding EST clones was obtained. The joined cDNA fragment (hpa), 1721 bp long (SEQ ID NO:9), contained an open reading frame which encodes a polypeptide of 543 amino acids (SEQ ID NO:10) with a calculated molecular weight of 61,192 daltons.

Cloning an extended 5′ sequence was enabled from the human SK-hep1 cell line by PCR amplification using the Marathon RACE. The 5′ extended sequence of the SK-hep1 hpa cDNA was assembled with the sequence of the hpa cDNA isolated from human placenta (SEQ ID NO:9). The assembled sequence contained an open reading frame, SEQ ID NOs: 13 and 15, which encodes, as shown in SEQ ID NOs:14 and 15, a polypeptide of 592 amino acids with a calculated molecular weight of 66,407 daltons.

The ability of the hpa gene product to catalyze degradation of heparan sulfate in an in vitro assay was examined by expressing the entire open reading frame of hpa in insect cells, using the Baculovirus expression system. Extracts and conditioned media of cells infected with virus containing the hpa gene, demonstrated a high level of heparan sulfate degradation activity both towards soluble ECM-derived HSPG and intact ECM. This degradation activity was inhibited by heparin, which is another substrate of heparanase. Cells infected with a similar construct containing no hpa gene had no such activity, nor did non-infected cells. The ability of heparanase expressed from the extended 5′ clone towards heparin was demonstrated in a mammalian expression system.

The expression pattern of hpa RNA in various tissues and cell lines was investigated using RT-PCR. It was found to be expressed only in tissues and cells previously known to have heparanase activity.

A panel of monochromosomal human/CHO and human/mouse somatic cell hybrids was used to localize the human heparanase gene to human chromosome 4. The newly isolated heparanase sequence can be used to identify a chromosome region harboring a human heparanase gene in a chromosome spread.

A human genomic library was screened and the human locus harboring the heparanase gene isolated, sequenced and characterized. Alternatively spliced heparanase mRNAs were identified and characterized. The human heparanase promoter has been isolated, identified and positively tested for activity. The mouse heparanase promoter has been isolated and identified as well. Antisense heparanase constructs were prepared and their influence on cells in vitro tested. A predicted heparanase active site was identified. And finally, the presence of sequences hybridizing with human heparanase sequences was demonstrated for a variety of mammalians and for an avian.

According to one aspect of the present invention there is provided an isolated nucleic acid comprising a genomic, complementary or composite polynucleotide sequence encoding a polypeptide having heparanase catalytic activity.

According to further features in preferred embodiments of the invention described below, the polynucleotide or a portion thereof is hybridizable with SEQ ID NOs: 9, 13, 42, 43 or a portion thereof at 68° C. in 6×SSC, 1% SDS, 5×Denharts, 10% dextran sulfate, 100 μg/ml salmon sperm DNA, and ³²p labeled probe and wash at 68° C. with 3×SSC and 0.1% SDS.

According to still further features in the described preferred embodiments the polynucleotide or a portion thereof is at least 60% identical with SEQ ID NOs: 9, 13, 42, 43 or portions thereof as determined using the Bestfit procedure of the DNA sequence analysis software package developed by the Genetic Computer Group (GCG) at the university of Wisconsin (gap creation penalty—12, gap extension penalty—4).

According to still further features in the described preferred embodiments the polypeptide is as set forth in SEQ ID NOs:10, 14, 44 or portions thereof.

According to still further features in the described preferred embodiments the polypeptide is at least 60% homologous to SEQ ID NOs:10, 14, 44 or portions thereof as determined with the Smith-Waterman algorithm, using the Bioaccelerator platform developed by Compugene (gapop: 10.0, gapext: 0.5, matrix: blosum62).

According to additional aspects of the present invention there are provided a nucleic acid construct (vector) comprising the isolated nucleic acid described herein and a host cell comprising the construct.

According to a further aspect of the present invention there is provided an antisense oligonucleotide comprising a polynucleotide or a polynucleotide analog of at least 10 bases being hybridizable in vivo, under. physiological conditions, with a portion of a polynucleotide strand encoding a polypeptide having heparanase catalytic activity.

According to an additional aspect of the present invention there is provided a method of in vivo downregulating heparanase activity comprising the step of in vivo administering the antisense oligonucleotide herein described.

According to yet an additional aspect of the present invention there is provided a pharmaceutical composition comprising the antisense oligonucleotide herein described and a pharmaceutically acceptable carrier.

According to still an additional aspect of the present invention there is provided a ribozyme comprising the antisense oligonucleotide described herein and a ribozyme sequence.

According to a further aspect of the present invention there is provided an antisense nucleic acid construct comprising a promoter sequence and a polynucleotide sequence directing the synthesis of an antisense RNA sequence of at least 10 bases being hybridizable in vivo, under physiological conditions, with a portion of a polynucleotide strand encoding a polypeptide having heparanase catalytic activity.

According to further features in preferred embodiments of the invention described below, the polynucleotide strand encoding the polypeptide having heparanase catalytic activity is as set forth in SEQ ID NOs: 9, 13, 42 or 43.

According to still further features in the described preferred embodiments the polypeptide having heparanase catalytic activity is as set forth in SEQ ID NOs: 10, 14 or 44.

According to still a further aspect of the present invention there is provided a method of in vivo downregulating heparanase activity comprising the step of in vivo administering the antisense nucleic acid construct herein described.

According to yet a further aspect of the present invention there is provided a pharmaceutical composition comprising the antisense nucleic acid construct herein described and a pharmaceutically acceptable carrier.

According to a further aspect of the present invention there is provided a nucleic acid construct comprising a polynucleotide sequence functioning as a promoter, the polynucleotide sequence is derived from SEQ ID NO:42 and includes at least nucleotides 2535-2635 thereof or from SEQ ID NO:43 and includes at least nucleotides 320-420.

According to a further aspect of the present invention there is provided a method of expressing a polynucleotide sequence comprising the step of ligating the polynucleotide sequence into the nucleic acid construct described above, downstream of the polynucleotide sequence derived from SEQ ID NOs:42 or 43.

According to a further aspect of the present invention there is provided a recombinant protein comprising a polypeptide having heparanase catalytic activity.

According to further features in preferred embodiments of the invention described below, the polypeptide includes at least a portion of SEQ ID NOs:10, 14 or 44.

According to still further features in the described preferred embodiments the protein is encoded by a polynucleotide hybridizable with SEQ ID NOs: 9, 13, 42, 43 or a portion thereof at 68° C. in 6×SSC, 1% SDS, 5×Denharts, 10% dextran sulfate, 100 μg/ml salmon sperm DNA, and ³²p labeled probe and wash at 68° C. with 3×SSC and 0.1% SDS.

According to still further features in the described preferred embodiments the protein is encoded by a polynucleotide at least 60% identical with SEQ ID NOs: 9, 13, 42, 43 or portions thereof as determined using the Bestfit procedure of the DNA sequence analysis software package developed by the Genetic Computer Group (GCG) at the university of Wisconsin (gap creation penalty—12, gap extension penalty—4).

According to a further aspect of the present invention there is provided a pharmaceutical composition comprising, as an active ingredient, the recombinant protein herein described.

According to a further aspect of the present invention there is provided a method of identifying a chromosome region harboring a heparanase gene in a chromosome spread comprising the steps of (a) hybridizing the chromosome spread with a tagged polynucleotide probe encoding heparanase; (b) washing the chromosome spread, thereby removing excess of non-hybridized probe; and (c) searching for signals associated with the hybridized tagged polynucleotide probe, wherein detected signals being indicative of a chromosome region harboring a heparanase gene.

According to a further aspect of the present invention there is provided a method of in vivo eliciting anti-heparanase antibodies comprising the steps of administering a nucleic acid construct including a polynucleotide segment corresponding to at least a portion of SEQ ID NOs:9, 13 or 43 and a promoter for directing the expression of said polynucleotide segment in vivo. Accordingly, there is provided also a DNA vaccine for in vivo eliciting anti-heparanase antibodies comprising a nucleic acid construct including a polynucleotide segment corresponding to at least a portion of SEQ ID NOs:9, 13 or 43 and a promoter for directing the expression of said polynucleotide segment in vivo.

The present invention can be used to develop new drugs to inhibit tumor cell metastasis, inflammation and autoimmunity. The identification of the hpa gene encoding for heparanase enzyme enables the production of a recombinant enzyme in heterologous expression systems. Additional features, advantages, uses and applications of the present invention in biological science and in diagnostic and therapeutic medicine are described hereinafter.

### BRIEF DESCRIPTION OF THE DRAWINGS

The invention herein described, by way of example only, with reference to the accompanying drawings, wherein:

FIG. 1 presents nucleotide sequence and deduced amino acid sequence of hpa cDNA. A single nucleotide difference at position 799 (A to T) between the EST (Expressed Sequence Tag) and the PCR amplified cDNA (reverse transcribed RNA) and the resulting amino acid substitution (Tyr to Phe) are indicated above and below the substituted unit, respectively. Cysteine residues and the poly adenylation consensus sequence are underlined. The asterisk denotes the stop codon TGA.

FIG. 2 demonstrates degradation of soluble sulfate labeled HSPG substrate by lysates of High Five cells infected with pFhpa2 virus. Lysates of High Five cells that were infected with pFhpa2 virus (•) or control pF2 virus (□) were incubated (18 h, 37° C.) with sulfate labeled ECM-derived soluble HSPG (peak I). The incubation medium was then subjected to gel filtration on Sepharose 6B. Low molecular weight HS degradation fragments (peak II) were produced only during incubation with the pFhpa2 infected cells, but there was no degradation of the HSPG substrate ( [symbol] ) by lysates of pF2 infected cells.

FIGS. 3a-b demonstrate degradation of soluble sulfate labeled HSPG substrate by the culture medium of pFhpa2 and pFhpa4 infected cells. Culture media of High Five cells infected with pFhpa2 (3a) or pFhpa4 (3b) viruses (•), or with control viruses (□) were incubated (18 h, 37° C.) with sulfate labeled ECM-derived soluble HSPG (peak I, [symbol] ). The incubation media were then subjected to gel filtration on Sepharose 6B. Low molecular weight HS degradation fragments (peak II) were produced only during incubation with the hpa gene containing viruses. There was no degradation of the HSPG substrate by the culture medium of cells infected with control viruses.

FIG. 4 presents size fractionation of heparanase activity expressed by pFhpa2 infected cells. Culture medium of pFhpa2 infected High Five cells was applied onto a 50 kDa cut-off membrane. Heparanase activity (conversion of the peak I substrate, ( [symbol] ) into peak II HS degradation fragments) was found in the high (>50 kDa) (•),but not low (<50 kDa) (∘) molecular weight compartment.

FIGS. 5a-b demonstrate the effect of heparin on heparanase activity expressed by pFhpa2 and pFhpa4 infected High Five cells. Culture media of pFhpa2 (5a) and pFhpa4 (5b) infected High Five cells were incubated (18 h, 37° C.) with sulfate labeled ECM-derived soluble HSPG (peak I, [symbol] ) in the absence (•) or presence (Δ) of 10 μg/ml heparin. Production of low molecular weight HS degradation fragments was completely abolished in the presence of heparin, a potent inhibitor of heparanase activity (6, 7).

FIGS. 6a-b demonstrate degradation of sulfate labeled intact ECM by virus infected High Five and Sf21 cells. High Five (6a) and Sf21 (6b) cells were plated on sulfate labeled ECM and infected (48 h, 28° C.) with pFhpa4 (•) or control pF1 (□) viruses. Control non-infected Sf21 cells (▴) were plated on the labeled ECM as well. The pH of the cultured medium was adjusted to 6.0-6.2 followed by 24 h incubation at 37° C. Sulfate labeled material released into the incubation medium was analyzed by gel filtration on Sepharose 6B. HS degradation fragments were produced only by cells infected with the hpa containing virus.

FIGS. 7a-b demonstrate degradation of sulfate labeled intact ECM by virus infected cells. High Five (7a) and Sf21 (7b) cells were plated on sulfate labeled ECM and infected (48 h, 28° C.) with pFhpa4 (•) or control pF1 (□) viruses. Control non-infected Sf21 cells (▴) were plate on labeled ECM as well. The pH of the cultured medium was adjusted to 6.0-6.2, followed by 48 h incubation at 28° C. Sulfate labeled degradation fragments released into the incubation medium was analyzed by gel filtration on Sepharose 6B. HS degradation fragments were produced only by cells infected with the hpa containing virus.

FIGS. 8a-b demonstrate degradation of sulfate labeled intact ECM by the culture medium of pFhpa4 infected cells. Culture media of High Five (8a) and Sf21 (8b) cells that were infected with pFhpa4 (•) or control pF1 (□) viruses were incubated (48 h, 37° C., pH 6.0) with intact sulfate labeled ECM. The ECM was also incubated with the culture medium of control non-infected Sf21 cells (▴). Sulfate labeled material released into the reaction mixture was subjected to gel filtration analysis. Heparanase activity was detected only in the culture medium of pFhpa4 infected cells.

FIGS. 9a-b demonstrate the effect of heparin on heparanase activity in the culture medium of pFhpa4 infected cells. Sulfate labeled ECM was incubated (24 h, 37° C., pH 6.0) with culture medium of pFhpa4 infected High Five (9a) and Sf21 (9b) cells in the absence (•) or presence (Δ) of 10 μg/ml heparin. Sulfate labeled material released into the incubation medium was subjected to gel filtration on Sepharose 6B. Heparanase activity (production of peak II HS degradation fragments) was completely inhibited in the presence of heparin.

FIGS. 10a-b demonstrate purification of recombinant heparanase on heparin-Sepharose. Culture medium of Sf21 cells infected with pFhpa4 virus was subjected to heparin-Sepharose chromatography. Elution of fractions was performed with 0.35-2 M NaCl gradient ( [symbol] ). Heparanase activity in the eluted fractions is demonstrated in FIG. 10a (•). Fractions 15-28 were subjected to 15% SDS-polyacrylamide gel electrophoresis followed by silver nitrate staining. A correlation is demonstrated between a major protein band (MW˜63,000) in fractions 19-24 and heparanase activity.

FIGS. 11a-b demonstrate purification of recombinant heparanase on a Superdex 75 gel filtration column. Active fractions eluted from heparin-Sepharose (FIG. 10a) were pooled, concentrated and applied onto Superdex 75 FPLC column. Fractions were collected and aliquots of each fraction were tested for heparanase activity (∘, FIG. 11a) and analyzed by SDS-polyacrylamide gel electrophoresis followed by silver nitrate staining (FIG. 11b). A correlation is seen between the appearance of a major protein band (MW˜63,000) in fractions 4-7 and heparanase activity.

FIGS. 12a-e demonstrate expression of the hpa gene by RT-PCR with total RNA from human embryonal tissues (12a), human extra-embryonal tissues (12b) and cell lines from different origins (12c-e). RT-PCR products using hpa specific primers (I), primers for GAPDH housekeeping gene (II), and control reactions without reverse transcriptase demonstrating absence of genomic DNA or other contamination in RNA samples (III). M-DNA molecular weight marker VI (Boehringer Mannheim). For 12a: lane 1—neutrophil cells (adult), lane 2—muscle, lane 3—thymus, lane 4—heart, lane 5—adrenal. For 12b: lane 1—kidney, lane 2—placenta (8 weeks), lane 3—placenta (11 weeks), lanes 4-7—mole (complete hydatidiform mole), lane 8—cytotrophoblast cells (freshly isolated), lane 9—cytotrophoblast cells (1.5 h in vitro), lane 10—cytotrophoblast cells (6 h in vitro), lane 11—cytotrophoblast cells (18 h in vitro), lane 12—cytotrophoblast cells (48 h in vitro). For 12c: lane 1—JAR bladder cell line, lane 2—NCITT testicular tumor cell line, lane 3—SW-480 human hepatoma cell line, lane 4—HTR (cytotrophoblasts transformed by SV40), lane 5—HPTLP-I hepatocellular carcinoma cell line, lane 6—EJ-28 bladder carcinoma cell line. For 12d: lane 1—SK-hep-1 human hepatoma cell line, lane 2—DAMI human megakaryocytic cell line, lane 3—DAMI cell line+PMA, lane 4—CHRF cell line+PMA, lane 5—CHRF cell line. For 12e: lane 1—ABAE bovine aortic endothelial cells, lane 2-1063 human ovarian cell line, lane 3—human breast carcinoma MDA435 cell line, lane 4—human breast carcinoma MDA231 cell line.

FIG. 13 presents a comparison between nucleotide sequences of the human hpa and a mouse EST cDNA fragment (SEQ ID NO:12) which is 80% homologous to the 3′ end (starting at nucleotide 1066 of SEQ ID NO:9) of the human hpa. The aligned termination codons are underlined.

FIG. 14 demonstrates the chromosomal localization of the hpa gene. PCR products of DNA derived from somatic cell hybrids and of genomic DNA of hamster, mouse and human of were separated on 0.7% agarose gel following amplification with hpa specific primers. Lane 1—Lambda DNA digested with BstEII, lane 2—no DNA control, lanes 3-29, PCR amplification products. Lanes 3-5—human, mouse and hamster genomic DNA, respectively. Lanes 6-29, human monochromosomal somatic cell hybrids representing chromosomes 1-22 and X and Y, respectively. Lane 30—Lambda DNA digested with BstEII. An amplification product of approximately 2.8 Kb is observed only in lanes 5 and 9, representing human genomic DNA and DNA derived from cell hybrid carrying human chromosome 4, respectively. These results demonstrate that the hpa gene is localized in human chromosome 4.

FIG. 15 demonstrates the genomic exon-intron structure of the human hpa locus (top) and the relative positions of the lambda clones used as sequencing templates to sequence the locus (below). The vertical rectangles represent exons (E) and the horizontal lines therebetween represent introns (I), upstream (U) and downstream (D) regions. Continuous lines represent DNA fragments, which were used for sequence analysis. The discontinuous line in lambda 6 represent a region, which overlaps with lambda 8 and hence was not analyzed. The plasmid contains a PCR product, which bridges the gap between L3 and L6.

FIGS. 16a-p presents the nucleotide sequence of the genomic region of the hpa gene. Exon sequences appear in upper case and intron sequences in lower case. The deduced amino acid sequence of the exons is printed below the nucleotide sequence. Two predicted transcription start sites are shown in bold.

FIG. 17 presents an alignment of the amino acid sequences of human heparanase, mouse and partial sequences of rat homologues. The human and the mouse sequences were determined by sequence analysis of the isolated cDNAs. The rat sequence is derived from two different EST clones, which represent two different regions (5′ and 3′) of the rat hpa cDNA. The human sequence and the amino acids in the mouse and rat homologues, which are identical to the human sequence, appear in bold.

FIG. 18 presents a heparanase Zoo blot. Ten micrograms of genomic DNA from various sources were digested with EcoRI and separated on 0.7% agarose—TBE gel. Following electrophoresis, the was gel treated with HCl and than with NaOH and the DNA fragments were downward transferred to a nylon membrane (Hybond N+, Amersham) with 0.4 N NaOH. The membrane was hybridized with a 1.6 Kb DNA probe that contained the entire hpa cDNA. Lane order: H—Human; M—Mouse; Rt—Rat; P—Pig; Cw—Cow; Hr—Horse; S—Sheep; Rb—Rabbit; D—Dog; Ch—Chicken; F—Fish. Size markers (Lambda BstEII) are shown on the left.

FIG. 19 demonstrates the secondary structure prediction for heparanase performed using the PHD server—Profile network Prediction Heidelberg. H—helix, E—extended (beta strand), The glutamic acid predicted as the proton donor is marked by asterisk and the possible nucleophiles are underlined.

### DESCRIPTION OF THE PREFERRED EMBODIMENTS

The present invention is of a polynucleotide or nucleic acid, referred to hereinbelow interchangeably as hpa, hpa cDNA or hpa gene or identified by its SEQ ID NOs, encoding a polypeptide having heparanase activity, vectors or nucleic acid constructs including same and which are used for over-expression or antisense inhibition of heparanase, genetically modified cells expressing same, recombinant protein having heparanase activity, antisense oligonucleotides and ribozymes for heparanase modulation, and heparanase promoter sequences which can be used to direct the expression of desired genes.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Cloning of the human and mouse hpa genes, cDNAs and genomic sequence (for human), encoding heparanase and expressing recombinant heparanase by transfected cells is reported herein. These are the first mammalian heparanase genes to be cloned.

A purified preparation of heparanase isolated from human hepatoma cells was subjected to tryptic digestion and microsequencing.

The YGPDVGQPR (SEQ ID NO:8) sequence revealed was used to screen EST databases for homology to the corresponding back translated DNA sequences. Two closely related EST sequences were identified and were thereafter found to be identical.

Both clones contained an insert of 1020 bp which includes an open reading frame of 973 bp followed by a 3′ untranslated region of 27 bp and a Poly A tail, whereas a translation start site was not identified.

Cloning of the missing 5′ end was performed by PCR amplification of DNA from placenta Marathon RACE cDNA composite using primers selected according to the EST clones sequence and the linkers of the composite.

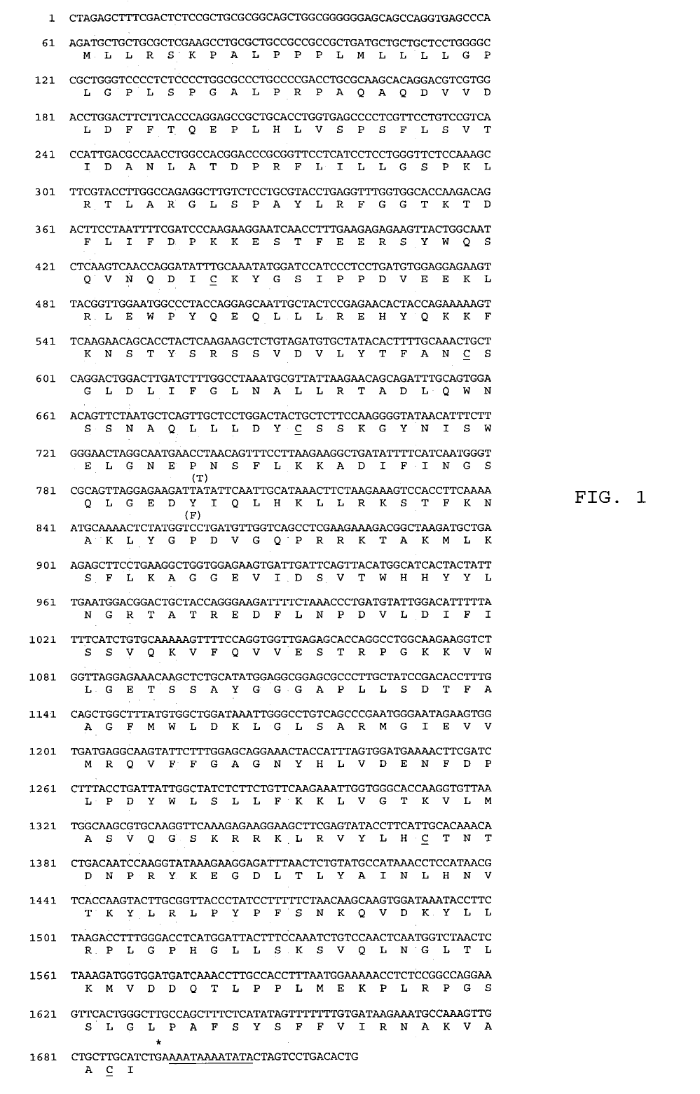

A 900 bp PCR fragment, partially overlapping with the identified 3′ encoding EST clones was obtained. The joined cDNA fragment (hpa), 1721 bp long (SEQ ID NO:9), contained an open reading frame which encodes, as shown in FIG. 1 and SEQ ID NO:11, a polypeptide of 543 amino acids, (SEQ ID NO:10) with a calculated molecular weight of 61,192 daltons.

A single nucleotide difference at position 799 (A to T) between the EST clones and the PCR amplified cDNA was observed. This difference results in a single amino acid substitution (Tyr to Phe) (FIG. 1). Furthermore, the published EST sequences contained an unidentified nucleotide, which following DNA sequencing of both the EST clones was resolved into two nucleotides (G and C at positions 1630 and 1631 in SEQ ID NO:9, respectively).

The ability of the hpa gene product to catalyze degradation of heparan sulfate in an in vitro assay was examined by expressing the entire open reading frame in insect cells, using the Baculovirus expression system.

Extracts and conditioned media of cells infected with virus containing the hpa gene, demonstrated a high level of heparan sulfate degradation activity both towards soluble ECM-derived HSPG and intact ECM, which was inhibited by heparin, while cells infected with a similar construct containing no hpa gene had no such activity, nor did non-infected cells.

The expression pattern of hpa RNA in various tissues and cell lines was investigated using RT-PCR. It was found to be expressed only in tissues and cells previously known to have heparanase activity.

Cloning an extended 5′ sequence was enabled from the human SK-hep1 cell line by PCR amplification using the Marathon RACE. The 5′ extended sequence of the SK-hep1 hpa cDNA was assembled with the sequence of the hpa cDNA isolated from human placenta (SEQ ID NO:9). The assembled sequence contained an open reading frame, SEQ ID NOs: 13 and 15, which encodes, as shown in SEQ ID NOs:14 and 15, a polypeptide of 592 amino acids, with a calculated molecular weight of 66,407 daltons. This open reading frame was shown to direct the expression of catalytically active heparanase in a mammalian cell expression system. The expressed heparanase was detectable by anti heparanase antibodies in Western blot analysis.

A panel of monochromosomal human/CHO and human/mouse somatic cell hybrids was used to localize the human heparanase gene to human chromosome 4. The newly isolated heparanase sequence can therefore be used to identify a chromosome region harboring a human heparanase gene in a chromosome spread.

The hpa cDNA was then used as a probe to screen a a human genomic library. Several phages were positive. These phages were analyzed and were found to cover most of the hpa locus, except for a small portion which was recovered by bridging PCR. The hpa locus covers about 50,000 bp. The hpa gene-includes 12 exons separated by 11 introns.

RT-PCR performed on a variety of cells revealed alternatively spliced hpa transcripts.

The amino acid sequence of human heparanase was used to search for homologous sequences in the DNA and protein databases. Several human EST's were identified, as well as mouse sequences highly homologous to human heparanase. The following mouse EST's were identified AA177901, AA674378, AA67997, AA047943, AA690179, AI122034, all sharing an identical sequence and correspond to amino acids 336-543 of the human heparanase sequence. The entire mouse heparanase cDNA was cloned, based on the nucleotide sequence of the mouse EST's using Marathon cDNA libraries. The mouse and the human hpa genes share an average homology of 78% between the nucleotide sequences and 81% similarity between the deduced amino acid sequences. hpa homologous sequences from rat were also uncovered (EST's AI060284 and AI237828).

Homology search of heparanase amino acid sequence against the DNA and the protein databases and prediction of its protein secondary structure enabled to identify candidate amino acids that participate in the heparanase active site.

Expression of hpa antisense in mammalian cell lines resulted in about five fold decrease in the number of recoverable cells as compared to controls.

Human Hpa cDNA was shown to hybridize with genomic DNAs of a variety of mammalian species and with an avian.

The human and mouse hpa promoters were identified and the human promoter was tested positive in directing the expression of a reporter gene.

Thus, according to the present invention there is provided an isolated nucleic acid comprising a genomic, complementary or composite polynucleotide sequence encoding a polypeptide having heparanase catalytic activity.

The phrase “composite polynucleotide sequence” refers to a sequence which includes exonal sequences required to encode the polypeptide having heparanase activity, as well as any number of intronal sequences. The intronal sequences can be of any source and typically will include conserved splicing signal sequences. Such intronal sequences may further include cis acting expression regulatory elements.

The term “heparanase catalytic activity” or its equivalent term “heparanase activity” both refer to a mammalian endoglycosidase hydrolyzing activity which is specific for heparan or heparan sulfate proteoglycan substrates, as opposed to the activity of bacterial enzymes (heparinase I, II and III) which degrade heparin or heparan sulfate by means of β-elimination (37).

According to a preferred embodiment of the present invention the polynucleotide or a portion thereof is hybridizable with SEQ ID NOs: 9, 13, 42, 43 or a portion thereof at 68° C. in 6×SSC, 1% SDS, 5×Denharts, 10% dextran sulfate, 100 μg/ml salmon sperm DNA, and ³²p labeled probe and wash at 68° C. with 3, 2, 1, 0.5 or 0.1×SSC and 0.1% SDS.

According to another preferred embodiment of the present invention the polynucleotide or a portion thereof is at least 60%, preferably at least 65%, more preferably at least 70%, more preferably at least 75%, more preferably at least 80%, more preferably at least 85%, more preferably at least 90%, most preferably, 95-100% identical with SEQ ID NOs: 9, 13, 42, 43 or portions thereof as determined using the Bestfit procedure of the DNA sequence analysis software package developed by the Genetic Computer Group (GCG) at the university of Wisconsin (gap creation penalty—12, gap extension penalty—4—which are the default parameters).

According to another preferred embodiment of the present invention the polypeptide encoded by the polynucleotide sequence is as set forth in SEQ ID NOs:10, 14, 44 or portions thereof having heparanase catalytic activity. Such portions are expected to include amino acids Asp-Glu 224-225 (SEQ ID NO:10), which can serve as proton donors and glutamic acid 343 or 396 which can serve as a nucleophile.

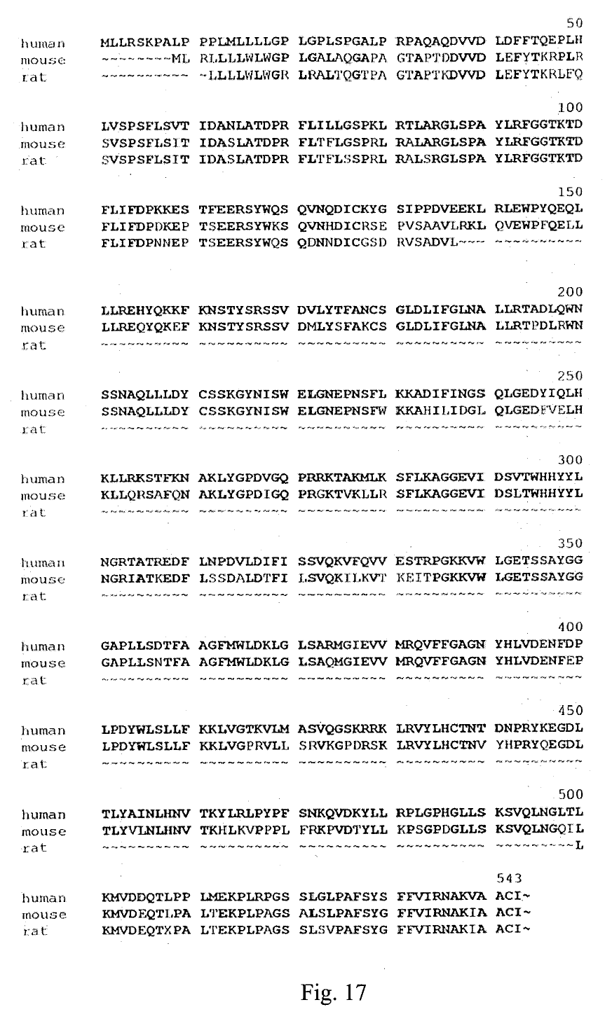

According to another preferred embodiment of the present invention the polypeptide encoded by the polynucleotide sequence is at least 60%, preferably at least 65%, more preferably at least 70%, more preferably at least 75%, more preferably at least 80%, more preferably at least 85%, more preferably at least 90%, most preferably, 95-100% homologous (both similar and identical acids) to SEQ ID NOs:10, 14, 44 or portions thereof as determined with the Smith-Waterman algorithm, using the Bioaccelerator platform developed by Compugene (gapop: 10.0, gapext: 0.5, matrix: blosum62, see also the description to FIG. 17).

Further according to the present invention there is provided a nucleic acid construct comprising the isolated nucleic acid described herein. The construct may and preferably further include an origin of replication and trans regulatory elements, such as promoter and enhancer sequences. The construct or vector can be of any type. It may be a phage which infects bacteria or a virus which infects eukaryotic cells. It may also be a plasmid, phagemid, cosmid, bacmid or an artificial chromosome.

Further according to the present invention there is provided a host cell comprising the nucleic acid construct described herein. The host cell can be of any type. It may be a prokaryotic cell, an eukaryotic cell, a cell line, or a cell as a portion of an organism. The polynucleotide encoding heparanase can be permanently or transiently present in the cell. In other words, genetically modified cells obtained following stable or transient transfection, transformation or transduction are all within the scope of the present invention. The polynucleotide can be present in the cell in low copy (say 1-5 copies) or high copy number (say 5-50 copies or more). It may be integrated in one or more chromosomes at any location or be present as an extrachromosomal material.

The present invention is further directed at providing a heparanase over-expression system which includes a cell overexpressing heparanase catalytic activity. The cell may be a genetically modified host cell transiently or stably transfected or transformed with any suitable vector which includes a polynucleotide sequence encoding a polypeptide having heparanase activity and a suitable promoter and enhancer sequences to direct over-expression of heparanase. However, the overexpressing cell may also be a product of an insertion (e.g., via homologous recombination) of a promoter and/or enhancer sequence downstream to the endogenous heparanase gene of the expressing cell, which will direct over-expression from the endogenous gene.

The term “over-expression” as used herein in the specification and claims below refers to a level of expression which is higher than a basal level of expression typically characterizing a given cell under otherwise identical conditions.

According to another aspect the present invention provides an antisense oligonucleotide comprising a polynucleotide or a polynucleotide analog of at least about 10, preferably from about 11 to about 15, more preferably about 16 or about 17, more preferably about 18, more preferably from about 19 to about 25, more preferably from about 26 to about 35, and optionally and most preferably from about 35 to about 100 bases being hybridizable in vivo, under physiological conditions, with a portion of a polynucleotide strand encoding a polypeptide having heparanase catalytic activity. The antisense oligonucleotide can be used for downregulating heparanase activity by in vivo administration thereof to a patient. As such, the antisense oligonucleotide according to the present invention can be used to treat types of cancers which are characterized by impaired (over) expression of heparanase, and are dependent on the expression of heparanase for proliferating or forming metastases.

The antisense oligonucleotide can be DNA or RNA or even include nucleotide analogs, examples of which are provided in the Background section hereinabove. The antisense oligonucleotide according to the present invention can be synthetic and is preferably prepared by solid phase synthesis. In addition, it can be of any desired length which still provides specific base pairing (e.g., 8 or 10, preferably more, nucleotides long) and it can include mismatches that do not hamper base pairing under physiological conditions.

Further according to the present invention there is provided a pharmaceutical composition comprising the antisense oligonucleotide herein described and a pharmaceutically acceptable carrier. The carrier can be, for example, a liposome loadable with the antisense oligonucleotide.

According to a preferred embodiment of the present invention the antisense oligonucleotide further includes a ribozyme sequence. The ribozyme sequence serves to cleave a heparanase RNA molecule to which the antisense oligonucleotide binds, to thereby downregulate heparanase expression.

Further according to the present invention there is provided an antisense nucleic acid construct comprising a promoter sequence and a polynucleotide sequence directing the synthesis of an antisense RNA sequence of at least 10 bases being hybridizable in vivo, under physiological conditions, with a portion of a polynucleotide strand encoding a polypeptide having heparanase catalytic activity. Like the antisense oligonucleotide, the antisense construct can be used for downregulating heparanase activity by in vivo administration thereof to a patient. As such, the antisense construct, like the antisense oligonucleotide, according to the present invention can be used to treat types of cancers which are characterized by impaired (over) expression of heparanase, and are dependent on the expression of heparanase for proliferating or forming metastases.

Thus, further according to the present invention there is provided a pharmaceutical composition comprising the antisense construct herein described and a pharmaceutically acceptable carrier. The carrier can be, for example, a liposome loadable with the antisense construct. Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable. Coated condoms, stents, active pads, and other medical devices may also be useful. Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include, but are not limited to, sterile aqueous solutions which may also contain buffers, diluents and other suitable additives.

Dosing is dependent on severity and responsiveness of the condition to be treated, but will normally be one or more doses per day, week or month with course of treatment lasting from several days to several months or until a cure is effected or a diminution of disease state is achieved. Persons ordinarily skilled in the art can easily determine optimum dosages, dosing methodologies and repetition rates.

Further according to the present invention there is provided a nucleic acid construct comprising a polynucleotide sequence functioning as a promoter, the polynucleotide sequence is derived from SEQ ID NO:42 and includes at least nucleotides 2135-2635, preferably 2235-2635, more preferably 2335-2635, more preferably 2435-2635, most preferably 2535-2635 thereof, or SEQ ID NO:43 and includes at least nucleotides 1-420, preferably 120-420, more preferably 220-420, most preferably 320-420, thereof. These nucleotides are shown in the example section that follows to direct the synthesis of a reporter gene in transformed cells. Thus, further according to the present invention there is provided a method of expressing a polynucleotide sequence comprising the step of ligating the polynucleotide sequence downstream to either of the promoter sequences described herein. Heparanase promoters can be isolated from a variety of mammalian an other species by cloning genomic regions present 5′ to the coding sequence thereof. This can be readily achievable by one ordinarily skilled in the art using the heparanase polynucleotides described herein, which are shown in the Examples section that follows to participate in efficient cross species hybridization.

Further according to the present invention there is provided a recombinant protein comprising a polypeptide having heparanase catalytic activity. The protein according to the present invention include modifications known as post translational modifications, including, but not limited to, proteolysis (e.g., removal of a signal peptide and of a pro- or preprotein sequence), methionine modification, glycosylation, alkylation (e.g., methylation), acetylation, etc. According to preferred embodiments the polypeptide includes at least a portion of SEQ ID NOs:10, 14 or 44, the portion has heparanase catalytic activity. According to preferred embodiments of the present invention the protein is encoded by any of the above described isolated nucleic acids. Further according to the present invention there is provided a pharmaceutical composition comprising, as an active ingredient, the recombinant protein described herein.

The recombinant protein may be purified by any conventional protein purification procedure close to homogeneity and/or be mixed with additives. The recombinant protein may be manufactured using any of the genetically modified cells described above, which include any of the expression nucleic acid constructs described herein. The recombinant protein may be in any form. It may be in a crystallized form, a dehydrated powder form or in solution. The recombinant protein may be useful in obtaining pure heparanase, which in turn may be useful in eliciting anti-heparanase antibodies, either poly or monoclonal antibodies, and as a screening active ingredient in an anti-heparanase inhibitors or drugs screening assay or system.

Further according to the present invention there is provided a method of identifying a chromosome region harboring a human heparanase gene in a chromosome spread. the method is executed implementing the following method steps, in which in a first step the chromosome spread (either interphase or metaphase spread) is hybridized with a tagged polynucleotide probe encoding heparanase. The tag is preferably a fluorescent tag. In a second step according to the method the chromosome spread is washed, thereby excess of non-hybridized probe is removed. Finally, signals associated with the hybridized tagged polynucleotide probe are searched for, wherein detected signals being indicative of a chromosome region harboring the human heparanase gene. One ordinarily skilled in the art would know how to use the sequences disclosed herein in suitable labeling reactions and how to use the tagged probes to detect, using in situ hybridization, a chromosome region harboring a human heparanase gene.

Further according to the present invention there is provided a method of in vivo eliciting anti-heparanase antibodies comprising the steps of administering a nucleic acid construct including a polynucleotide segment corresponding to at least a portion of SEQ ID NOs:9, 13 or 43 and a promoter for directing the expression of said polynucleotide segment in vivo. Accordingly; there is provided also a DNA vaccine for in vivo eliciting anti-heparanase antibodies comprising a nucleic acid construct including a polynucleotide segment corresponding to at least a portion of SEQ ID NOs:9, 13 or 43 and a promoter for directing the expression of said polynucleotide segment in vivo. The vaccine optionally further includes a pharmaceutically acceptable carrier, such as a virus, liposome or an antigen presenting cell. Alternatively, the vaccine is employed as a naked DNA vaccine

The present invention can be used to develop treatments for various diseases, to develop diagnostic assays for these diseases and to provide new tools for basic research especially in the fields of medicine and biology.

Specifically, the present invention can be used to develop new drugs to inhibit tumor cell metastasis, inflammation and autoimmunity. The identification of the hpa gene encoding for the heparanase enzyme enables the production of a recombinant enzyme in heterologous expression systems.

Furthermore, the present invention can be used to modulate bioavailability of heparin-binding growth factors, cellular responses to heparin-binding growth factors (e.g., bFGF, VEGF) and cytokines (e.g., IL-8), cell interaction with plasma lipoproteins, cellular susceptibility to viral, protozoa and some bacterial infections, and disintegration of neurodegenerative plaques. Recombinant heparanase offers a potential treatment for wound healing, angiogenesis, restenosis, atherosclerosis, inflammation, neurodegenerative diseases (such as, for example, Genstmann-Straussler Syndrome, Creutzfeldt-Jakob disease, Scrape and Alzheimer's disease) and certain viral and some bacterial and protozoa infections. Recombinant heparanase can be used to neutralize plasma heparin, as a potential replacement of protamine.

As used herein, the term “modulate” includes substantially inhibiting, slowing or reversing the progression of a disease, substantially ameliorating clinical symptoms of a disease or condition, or substantially preventing the appearance of clinical symptoms of a disease or condition. A “modulator” therefore includes an agent which may modulate a disease or condition. Modulation of viral, protozoa and bacterial infections includes any effect which substantially interrupts, prevents or reduces any viral, bacterial or protozoa activity and/or stage of the virus, bacterium or protozoon life cycle, or which reduces or prevents infection by the virus, bacterium or protozoon in a subject, such as a human or lower animal.

As used herein, the term “wound” includes any injury to any portion of the body of a subject including, but not limited to, acute conditions such as thermal burns, chemical burns, radiation burns, burns caused by excess exposure to ultraviolet radiation such as sunburn, damage to bodily tissues such as the perineum as a result of labor and childbirth, including injuries sustained during medical procedures such as episiotomies, trauma-induced injuries including cuts, those injuries sustained in automobile and other mechanical accidents, and those caused by bullets, knives and other weapons, and post-surgical injuries, as well as chronic conditions such as pressure sores, bedsores, conditions related to diabetes and poor circulation, and all types of acne, etc.

Anti-heparanase antibodies, raised against the recombinant enzyme, would be useful for immunodetection and diagnosis of micrometastases, autoimmune lesions and renal failure in biopsy specimens, plasma samples, and body fluids. Such antibodies may also serve as neutralizing agents for heparanase activity.

The genomic heparanase sequences described herein can be used to construct knock-in and knock-out constructs. Such constructs include a fragment of 10-20 Kb of a heparanase locus and a negative and a positive selection markers and can be used to provide heparanase knock-in and knock-out animal models by methods known to the skilled artisan. Such animal models can be used for studying the function of heparanase in developmental processes, and in normal as well as pathological processes. They can also serve as an experimental model for testing drugs and gene therapy protocols. The complementary heparanase sequence (cDNA) can be used to derive transgenic animals, overexpressing heparanase for same. Alternatively, if cloned in the antisense orientation, the complementary heparanase sequence (cDNA) can be used to derive transgenic animals under-expressing heparanase for same.

The heparanase promoter sequences described herein and other cis regulatory elements linked to the heparanase locus can be used to regulated the expression of genes. For example, these promoters can be used to direct the expression of a cytotoxic protein, such as TNF, in tumor cells. It will be appreciated that heparanase itself is abnormally expressed under the control of its own promoter and other cis acting elements in a variety of tumors, and its expression is correlated with metastasis. It is also abnormally highly expressed in inflammatory cells. The introns of the heparanase gene can be used for the same purpose, as it is known that introns, especially upstream introns include cis acting element which affect expression. A heparanase promoter fused to a reporter protein can be used to study/monitor its activity.

The polynucleotide sequences described herein can also be used to provide DNA vaccines which will elicit in vivo anti heparanase antibodies. Such vaccines can therefore be used to combat inflammatory reactions and cancer.

Antisense oligonucleotides derived according to the heparanase sequences described herein, especially such oligonucleotides supplemented with ribozyme activity, can be used to modulate heparanase expression. Such oligonucleotides can be from the coding region, from the introns or promoter specific. Antisense heparanase nucleic acid constructs can similarly function, as well known in the art.

The heparanase sequences described herein can be used to study the catalytic mechanism of heparanase. Carefully selected site directed mutagenesis can be employed to provide modified heparanase proteins having modified characteristics in terms of, for example, substrate specificity, sensitivity to inhibitors, etc.

While studying heparanase expression in a variety of cell types alternatively spliced transcripts were identified. Such transcripts if found characteristic of certain pathological conditions can be used as markers for such conditions. Such transcripts are expected to direct the synthesis of heparanases with altered functions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturers' specifications. These techniques and various other techniques are generally performed according to Sambrook et al., Molecular Cloning—A Laboratory Manual, Cold Spring Harbor Laboratory, Cold Spring Harbor, N.Y. (1989), which is incorporated herein by reference. Other general references are provided throughout this document. The procedures therein are believed to be well known in the art and are provided for the convenience of the reader. All the information contained therein is incorporated herein by reference.

The following protocols and experimental details are referenced in the Examples that follow:

Purification and characterization of heparanase from a human hepatoma cell line and human placenta: A human hepatoma cell line (Sk-hep-1) was chosen as a source for purification of a human tumor-derived heparanase. Purification was essentially as described in U.S. Pat. No. 5,362,641 to Fuks, which is incorporated by reference as if fully set forth herein. Briefly, 500 liter, 5×10¹¹ cells were grown in suspension and the heparanase enzyme was purified about 240,000 fold by applying the following steps: (i) cation exchange (CM-Sephadex) chromatography performed at pH 6.0, 0.3-1.4 M NaCl gradient; (ii) cation exchange (CM-Sephadex) chromatography performed at pH 7.4 in the presence of 0.1% CHAPS, 0.3-1.1 M NaCl gradient; (iii) heparin-Sepharose chromatography performed at pH 7.4 in the presence of 0.1% CHAPS, 0.35-1.1 M NaCl gradient; (iv) ConA-Sepharose chromatography performed at pH 6.0 in buffer containing 0.1% CHAPS and 1 M NaCl, elution with 0.25 M α-methyl mannoside; and (v) HPLC cation exchange (Mono-S) chromatography performed at pH 7.4 in the presence of 0.1% CHAPS, 0.25-1 M NaCl gradient.

Active fractions were pooled, precipitated with TCA and the precipitate subjected to SDS polyacrylamide gel electrophoresis and/or tryptic digestion and reverse phase HPLC. Tryptic peptides of the purified protein were separated by reverse phase HPLC (C8 column) and homogeneous peaks were subjected to amino acid sequence analysis.

The purified enzyme was applied to reverse phase HPLC and subjected to N-terminal amino acid sequencing using the amino acid sequencer (Applied Biosystems).

Cells: Cultures of bovine corneal endothelial cells (BCECs) were established from steer eyes as previously described (19, 38). Stock cultures were maintained in DMEM (1 g glucose/liter) supplemented with 10% newborn calf serum and 5% FCS. bFGF (1 ng/ml) was added every other day during the phase of active cell growth (13, 14).

Preparation of dishes coated with ECM: BCECs (second to fifth passage) were plated into 4-well plates at an initial density of 2×10⁵ cells/ml, and cultured in sulfate-free Fisher medium plus 5% dextran T-40 for 12 days. Na₂³⁵SO₄ (25 μCi/ml) was added on day 1 and 5 after seeding and the cultures were incubated with the label without medium change. The subendothelial ECM was exposed by dissolving (5 min., room temperature) the cell layer with PBS containing 0.5% Triton X-100 and 20 mM NH₄OH, followed by four washes with PBS. The ECM remained intact, free of cellular debris and firmly attached to the entire area of the tissue culture dish (19, 22).

To prepare soluble sulfate labeled proteoglycans (peak I material), the ECM was digested with trypsin (25 μg/ml, 6 h, 37° C.), the digest was concentrated by reverse dialysis and the concentrated material was applied onto a Sepharose 6B gel filtration column. The resulting high molecular weight material (Kav<0.2, peak I) was collected. More than 80% of the labeled material was shown to be composed of heparan sulfate proteoglycans (11, 39).

Heparanase activity: Cells (1×10⁶/35-mm dish), cell lysates or conditioned media were incubated on top of ³⁵S-labeled ECM (18 h, 37° C.) in the presence of 20 mM phosphate buffer (pH 6.2). Cell lysates and conditioned media were also incubated with sulfate labeled peak I material (10-20 μl). The incubation medium was collected, centrifuged (18,000×g, 4° C., 3 min.), and sulfate labeled material analyzed by gel filtration on a Sepharose CL-6B column (0.9×30 cm). Fractions (0.2 ml) were eluted with PBS at a flow rate of 5 ml/h and counted for radioactivity using Bio-fluor scintillation fluid. The excluded volume (V_{O}) was marked by blue dextran and the total included volume (Vₜ) by phenol red. The latter was shown to comigrate with free sulfate (7, 11, 23). Degradation fragments of HS side chains were eluted from Sepharose 6B at 0.5<Kav<0.8 (peak II) (7, 11, 23). A nearly intact HSPG released from ECM by trypsin—and, to a lower extent, during incubation with PBS alone—was eluted next to V_{O} (Kav<0.2, peak I). Recoveries of labeled material applied on the columns ranged from 85 to 95% in different experiments (11). Each experiment was performed at least three times and the variation of elution positions (Kav values) did not exceed +/−15%.

Cloning of hpa cDNA: cDNA clones 257548 and 260138 were obtained from the I.M.A.G.E Consortium (2130 Memorial Parkway SW, Hunstville, Ala. 35801). The cDNAs were originally cloned in EcoRI and NotI cloning sites in the plasmid vector pT3T7D-Pac. Although these clones are reported to be somewhat different, DNA sequencing demonstrated that these clones are identical to one another. Marathon RACE (rapid amplification of cDNA ends) human placenta (poly-A) cDNA composite was a gift of Prof. Yossi Shiloh of Tel Aviv University. This composite is vector free, as it includes reverse transcribed cDNA fragments to which double, partially single stranded adapters are attached on both sides. The construction of the specific composite employed is described in reference 39a.

Amplification of hp3 PCR fragment was performed according to the protocol provided by Clontech laboratories. The template used for amplification was a sample taken from the above composite. The primers used for amplification were:

First step: 5′-primer: AP1: 5′-CCATCCTAATACGACTCACT ATAGGGC-3′, SEQ ID NO:1; 3′-primer: HPL229: 5′-GTAGTGATGCCA TGTAACTGAATC-3′, SEQ ID NO:2.

Second step: nested 5′-primer: AP2: 5′-ACTCACTATAGGGCTCG AGCGGC-3′, SEQ ID NO:3; nested 3′-primer: HPL171: 5′-GCATCTTAGCCGTCTTTCTTCG-3′, SEQ ID NO:4. The HPL229 and HPL171 were selected according to the sequence of the EST clones. They include nucleotides 933-956 and 876-897 of SEQ ID NO:9, respectively.

PCR program was 94° C.-4 min., followed by 30 cycles of 94° C.-40 sec., 62° C.-1 min., 72° C.-2.5 min. Amplification was performed with Expand High Fidelity (Boehringer Mannheim). The resulting ca. 900 bp hp3 PCR product was digested with BfrI and PvuII. Clone 257548 (phpa1) was digested with EcoRI, followed by end filling and was then further digested with BfrI. Thereafter the PvuII—BfrI fragment of the hp3 PCR product was cloned into the blunt end—BfrI end of clone phpa1 which resulted in having the entire cDNA cloned in pT3T7-pac vector, designated phpa2.

RT-PCR: RNA was prepared using TRI-Reagent (Molecular research center Inc.) according to the manufacturer instructions. 1.25 μg were taken for reverse transcription reaction using MuMLV Reverse transcriptase (Gibco BRL) and Oligo (dT)₁₅ primer, SEQ ID NO:5, (Promega). Amplification of the resultant first strand cDNA was performed with Taq polymerase (Promega). The following primers were used:

HPU-355: 5′-TTCGATCCCAAGAAGGAATCAAC-3′, SEQ ID NO:6, nucleotides 372-394 in SEQ ID NOs:9 or 11.

HPL-229: 5′-GTAGTGATGCCATGTAACTGAATC-3′, SEQ ID NO:7, nucleotides 933-956 in SEQ ID NOs:9 or 11.

PCR program: 94° C.-4 min., followed by 30 cycles of 94° C.-40 sec., 62° C.-1 min., 72° C.-1 min.

Alternatively, total RNA was prepared from cell cultures using Tri-reagent (Molecular Research Center, Inc.) according to the manufacturer recommendation. Poly A+ RNA was isolated from total RNA using mRNA separator (Clontech). Reverse transcription was performed with total RNA using Superscript II (GibcoBRL). PCR was performed with Expand high fidelity (Boehringer Mannheim). Primers used for amplification were as follows:

| Hpu- | 5′-GAGCAGCCAGGTGAGCCCAAGAT-3′, | SEQ ID NO:24 |
|---|---|---|
| 685, | | |
| Hpu- | 5′-TTCGATCCCAAGAAGGAATCAAC-3′, | SEQ ID NO:25 |
| 355, | | |
| Hpu | 5′-AGCTCTGTAGATGTGCTATACAC-3′, | SEQ ID NO:26 |
| 565, | | |
| Hpl | 5′-TCAGATGCAAGCAGCAACTTTGGC-3′, | SEQ ID NO:27 |
| 967, | | |
| Hpl | 5′-GCATCTTAGCCGTCTTTCTTCG-3′, | SEQ ID NO:28 |
| 171, | | |
| Hpl | 5′-GTAGTGATGCCATGTAACTGAATC-3′, | SEQ ID NO:29 |
| 229, | | |

PCR reaction was performed as follows: 94° C. 3 minutes, followed by 32 cycles of 94° C. 40 seconds, 64° C. 1 minute, 72° C. 3 minutes, and one cycle 72° C., 7 minutes.

Expression of recombinant heparanase in insect cells: Cells, High Five and Sf21 insect cell lines were maintained as monolayer cultures in SF900II-SFM medium (GibcoBRL).

Recombinant Baculovirus: Recombinant virus containing the hpa gene was constructed using the Bac to Bac system (GibcoBRL). The transfer vector pFastBac was digested with SalI and NotI and ligated with a 1.7 kb fragment of phpa2 digested with XhoI and NotI. The resulting plasmid was designated pFasthpa2. An identical plasmid designated pFasthpa4 was prepared as a duplicate and both independently served for further experimentations. Recombinant bacmid was generated according to the instructions of the manufacturer with pFasthpa2, pFasthpa4 and with pFastBac. The latter served as a negative control. Recombinant bacmid DNAs were transfected into Sf21 insect cells. Five days after transfection recombinant viruses were harvested and used to infect High Five insect cells, 3×10⁶ cells in T-25 flasks. Cells were harvested 2-3 days after infection. 4×10⁶ cells were centrifuged and resuspended in a reaction buffer containing 20 mM phosphate citrate buffer, 50 mM NaCl. Cells underwent three cycles of freeze and thaw and lysates were stored at −80° C. Conditioned medium was stored at 4° C.

Partial purification of recombinant heparanase: Partial purification of recombinant heparanase was performed by heparin-Sepharose column chromatography followed by Superdex 75 column gel filtration. Culture medium (150 ml) of Sf21 cells infected with pFhpa4 virus was subjected to heparin-Sepharose chromatography. Elution of 1 ml fractions was performed with 0.35-2 M NaCl gradient in presence of 0.1% CHAPS and 1 mM DTT in 10 mM sodium acetate buffer, pH 5.0. A 25 μl sample of each fraction was tested for heparanase activity. Heparanase activity was eluted at the range of 0.65-1.1 M NaCl (fractions 18-26, FIG. 10a). 5 μl of each fraction was subjected to 15% SDS-polyacrylamide gel electrophoresis followed by silver nitrate staining. Active fractions eluted from heparin-Sepharose (FIG. 10a) were pooled and concentrated (×6) on YM3 cut-off membrane. 0.5 ml of the concentrated material was applied onto 30 ml Superdex 75 FPLC column equilibrated with 10 mM sodium acetate buffer, pH 5.0, containing 0.8 M NaCl, 1 mM DTT and 0.1% CHAPS. Fractions (0.56 ml) were collected at a flow rate of 0.75 ml/min. Aliquots of each fraction were tested for heparanase activity and were subjected to SDS-polyacrylamide gel electrophoresis followed by silver nitrate staining (FIG. 11b).

PCR amplification of genomic DNA: 94° C. 3 minutes, followed by 32 cycles of 94° C. 45 seconds, 64° C. 1 minute, 68° C. 5 minutes, and one cycle at 72° C., 7 minutes. Primers used for amplification of genomic DNA included:

| | |
|---|---|
| GHpu-L3 | |
| 5′-AGGCACCCTAGAGATGTTCCAG-3′, | SEQ ID NO:30 |
| GHpl-L6 | |
| 5′-GAAGATTTCTGTTTCCATGACGTG-3′,. | SEQ ID NO:31 |

Screening of genomic libraries: A human genomic library in Lambda phage EMBLE3 SP6/T7 (Clontech, Paulo Alto, Calif.) was screened. 5×10⁵ plaques were plated at 5×10⁴ pfu/plate on NZCYM agar/top agarose plates. Phages were absorbed on nylon membranes in duplicates (Qiagen). Hybridization was performed at 65° C. in 5×SSC, 5×Denhart's, 10% dextran sulfate, 100 μg/ml Salmon sperm, ³²p labeled probe (10⁶ cpm/ml). A 1.6 kb fragment, containing the entire hpa cDNA was labeled by random priming (Boehringer Mannheim). Following hybridization membranes were washed once with 2×SSC, 0.1% SDS at 65° C. for 20 minutes, and twice with 0.2×SSC, 0.1% SDS at 65° C. for 15 minutes. Hybridizing plaques were picked, and plated at 100 pfu/plate. Hybridization was performed as above and single isolated positive plaques were picked.

Phage DNA was extracted using a Lambda DNA extraction kit (Qiagen). DNA was digested with XhoI and EcoRI, separated on 0.7% agarose gel and transferred to nylon membrane Hybond N+ (Amersham). Hybridization and washes were performed as above.

cDNA Sequence analysis: Sequence determinations were performed with vector specific and gene specific primers, using an automated DNA sequencer (Applied Biosystems, model 373A). Each nucleotide was read from at least two independent primers.

Genomic sequence analysis: Large-scale sequencing was performed by Commonwealth Biotechnology Incorporation.

Isolation of mouse hpa: Mouse hpa cDNA was amplified from either Marathon ready cDNA library of mouse embryo or from mRNA isolated from mouse melanoma cell line BL6, using the Marathon RACE kit from Clontech. Both procedures were performed according to the manufacturer's recommendation.

Primers used for PCR amplification of mouse hpa:

| | |
|---|---|
| Mhp1773 | |
| 5′-CCACACTGAATGTAATACTGAAGTG-3′, | SEQ ID NO:32 |
| MHp1736 | |
| 5′-CGAAGCTCTGGAACTCGGCAAG-3′, | SEQ ID NO:33 |
| MHp183 | |
| 5′-GCCAGCTGCAAAGGTGTTGGAC-3′, | SEQ ID NO:34 |
| Mhp1152 | |
| 5′-AACACCTGCCTCATCACGACTTC-3′, | SEQ ID NO:35 |
| Mhp1114 | |
| 5′-GCCAGGCTGGCGTCGATGGTGA-3′, | SEQ ID NO:36 |
| MHp1103 | |
| 5′-GTCGATGGTGATGGACAGGAAC-3′, | SEQ ID NO:37 |
| Ap1 | |
| 5′-GTAATACGACTCACTATAGGGC-3′, | SEQ ID NO:38 |
| (Genome walker) | |
| Ap2 | |
| 5′-ACTATAGGGCACGCGTGGT-3′, | SEQ ID NO:39 |
| (Genome walker) | |
| Ap1 | |
| 5′-CCATCCTAATACGACTCACTATAGGGC-3′, | SEQ ID NO:40 |
| (Marathon RACE) | |
| Ap2 | |
| 5′-ACTCACTATAGGGCTCGAGCGGC-3′, | SEQ ID NO:41 |
| (Marathon RACE) | |

Southern analysis of genomic DNA: Genomic DNA was extracted from animal or from human blood using Blood and cell culture DNA maxi kit (Qiagene). DNA was digested with EcoRI, separated by gel electrophoresis and. transferred to a nylon membrane Hybond N+ (Amersham). Hybridization was performed at 68° C. in 6×SSC, 1% SDS, 5×Denharts, 10% dextran sulfate, 100 μg/ml salmon sperm DNA, and ³²p labeled probe. A 1.6 kb fragment, containing the entire hpa cDNA was used as a probe. Following hybridization, the membrane was washed with 3×SSC, 0.1% SDS, at 68° C. and exposed to X-ray film for 3 days. Membranes were then washed with 1×SSC, 0.1% SDS, at 68° C. and were reexposed for 5 days.

Construction of hpa promoter-GFP expression vector. Lambda DNA of phage L3, was digested with SacI and BglII, resulting in a 1712 bp fragment which contained the hpa promoter (877-2688 of SEQ ID NO:42). The pEGFP-1 plasmid (Clontech) was digested with BglII and SacI and ligated with the 1712 bp fragment of the hpa promoter sequence. The resulting plasmid was designated phpEGL. A second hpa promoter-GFP plasmid was constructed containing a shorter fragment of the hpa promoter region: phpEGL was digested with HindIII, and the resulting 1095 bp fragment (nucleotides 1593-2688 of SEQ ID NO:42) was ligated with HindIII digested pEGFP-1. The resulting plasmid was designated phpEGS.

Computer analysis of sequences: Homology searches were performed using several computer servers, and various databases. Blast 2.0 service, at the NCBI server was used to screen the protein database swplus and DNA databases such as GenBank, EMBL, and the EST databases. Blast 2.0 search was performed using the basic search option of the NCBI server. Sequence analysis and alignments were done using the DNA sequence analysis software package developed by the Genetic Computer Group (GCG) at the university of Wisconsin. Alignments of two sequences were performed using Bestfit (gap creation penalty—12, gap extension penalty—4). Protein homology search was performed with the Smith-Waterman algorithm, using the Bioaccelerator platform developed by Compugene. The protein database swplus was searched using the following parameters: gapop: 10.0, gapext: 0.5, matrix: blosum62. Blocks homology was performed using the Blocks WWW server developed at Fred Hutchinson Cancer Research Center in Seattle, Wash., USA. Secondary structure prediction was performed using the PHD server—Profile network Prediction Heidelberg. Fold recognition (threading) was performed using the UCLA-DOE structure prediction server. The method used for prediction was gonnet+predss. Alignment of three sequences was performed using the pileup application (gap creation penalty—5, gap extension penalty—1). Promoter analysis was performed using TSSW and TSSG programs (BCM Search Launcher Human Genome Center, Baylor College of Medicine, Houston Tex.).

### Example 1

Cloning of Human hpa cDNA

Purified fraction of heparanase isolated from human hepatoma cells (SK-hep-1) was subjected to tryptic digestion and microsequencing. EST (Expressed Sequence Tag) databases were screened for homology to the back translated DNA sequences corresponding to the obtained peptides. Two EST sequences (accession Nos. N41349 and N45367) contained a DNA sequence encoding the peptide YGPDVGQPR (SEQ ID NO:8). These two sequences were derived from clones 257548 and 260138 (I.M.A.G.E Consortium) prepared from 8 to 9 weeks placenta cDNA library (Soares). Both clones which were found to be identical contained an insert of 1020 bp which included an open reading frame (ORF) of 973 bp followed by a 3′ untranslated region of 27 bp and a Poly A tail. No translation start site (AUG) was identified at the 5′ end of these clones.

Cloning of the missing 5′ end was performed by PCR amplification of DNA from a placenta Marathon RACE cDNA composite. A 900 bp fragment (designated hp3), partially overlapping with the identified 3′ encoding EST clones was obtained.

The joined cDNA fragment, 1721 bp long (SEQ ID NO:9), contained an open reading frame which encodes, as shown in FIG. 1 and SEQ ID NO:11, a polypeptide of 543 amino acids (SEQ ID NO:10) with a calculated molecular weight of 61,192 daltons. The 3′ end of the partial cDNA inserts contained in clones 257548 and 260138 started at nucleotide G⁷²¹ of SEQ ID NO:9 and FIG. 1.

As further shown in FIG. 1, there was a single sequence discrepancy between the EST clones and the PCR amplified sequence, which led to an amino acid substitution from Tyr²⁴⁶ in the EST to Phe²⁴⁶ in the amplified cDNA. The nucleotide sequence of the PCR amplified cDNA fragment was verified from two independent amplification products. The new gene was designated hpa.

As stated above, the 3′ end of the partial cDNA inserts contained in EST clones 257548 and 260138 started at nucleotide 721 of hpa (SEQ ID NO:9). The ability of the hpa cDNA to form stable secondary structures, such as stem and loop structures involving nucleotide stretches in the vicinity of position 721 was investigated using computer modeling. It was found that stable stem and loop structures are likely to be formed involving nucleotides 698-724 (SEQ ID NO:9). In addition, a high GC content, up to 70%, characterizes the 5′ end region of the hpa gene, as compared to about only 40% in the 3′ region. These findings may explain the immature termination and therefore lack of 5′ ends in the EST clones.

To examine the ability of the hpa gene product to catalyze degradation of heparan sulfate in an in vitro assay the entire open reading frame was expressed in insect cells, using the Baculovirus expression system. Extracts of cells, infected with virus containing the hpa gene, demonstrated a high level of heparan sulfate degradation activity, while cells infected with a similar construct containing no hpa gene had no such activity, nor did non-infected cells. These results are further demonstrated in the following Examples.

### Example 2

Degradation of Soluble ECM-Derived HSPG

Monolayer cultures of High Five cells were infected (72 h, 28° C.) with recombinant Bacoluvirus containing the pFasthpa plasmid or with control virus containing an insert free plasmid. The cells were harvested and lysed in heparanase reaction buffer by three cycles of freezing and thawing. The cell lysates were then incubated (18 h, 37° C.) with sulfate labeled, ECM-derived HSPG (peak I), followed by gel filtration analysis (Sepharose 6B) of the reaction mixture.

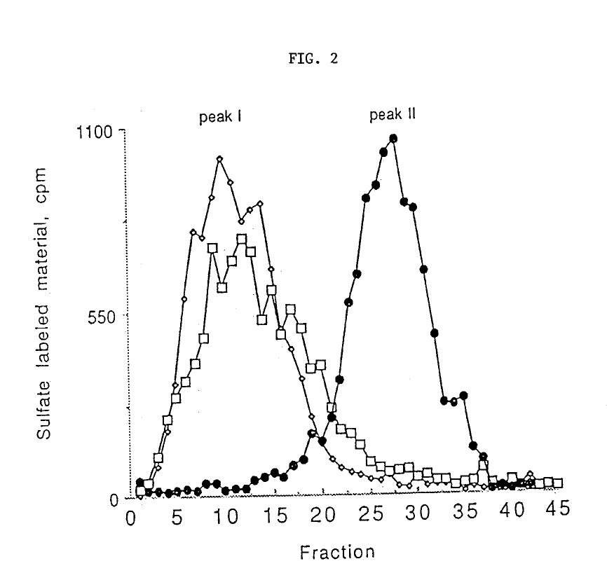

As shown in FIG. 2, the substrate alone included almost entirely high molecular weight (Mr) material eluted next to V_{O} (peak I, fractions 5-20, Kav<0.35). A similar elution pattern was obtained when the HSPG substrate was incubated with lysates of cells that were infected with control virus. In contrast, incubation of the HSPG substrate with lysates of cells infected with the hpa containing virus resulted in a complete conversion of the high Mr substrate into low Mr labeled degradation fragments (peak II, fractions 22-35, 0.5<Kav<0.75).

Fragments eluted in peak II were shown to be degradation products of heparan sulfate, as they were (i) 5- to 6-fold smaller than intact heparan sulfate side chains (Kav approx. 0.33) released from ECM by treatment with either alkaline borohydride or papain; and (ii) resistant to further digestion with papain or chondroitinase ABC, and susceptible to deamination by nitrous acid (6, 11). Similar results (not shown) were obtained with Sf21 cells. Again, heparanase activity was detected in cells infected with the hpa containing virus (pFhpa), but not with control virus (pF). This result was obtained with two independently generated recombinant viruses. Lysates of control not infected High Five cells failed to degrade the HSPG substrate.

In subsequent experiments, the labeled HSPG substrate was incubated with medium conditioned by infected High Five or Sf21 cells.

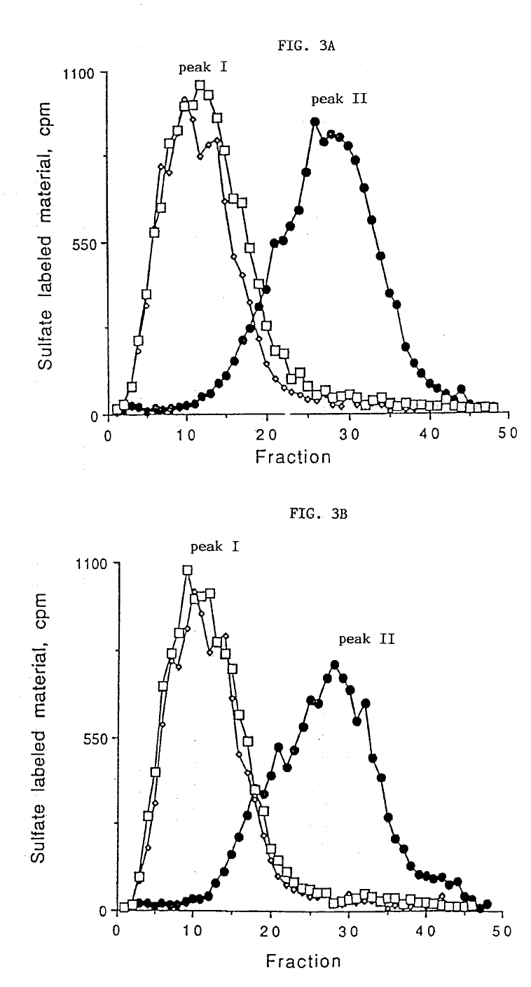

As shown in FIGS. 3a-b, heparanase activity, reflected by the conversion of the high Mr peak I substrate into the low Mr peak II which represents HS degradation fragments, was found in the culture medium of cells infected with the pFhpa2 or pFhpa4 viruses, but not with the control pF1 or pF2 viruses. No heparanase activity was detected in the culture medium of control non-infected High Five or Sf21 cells.

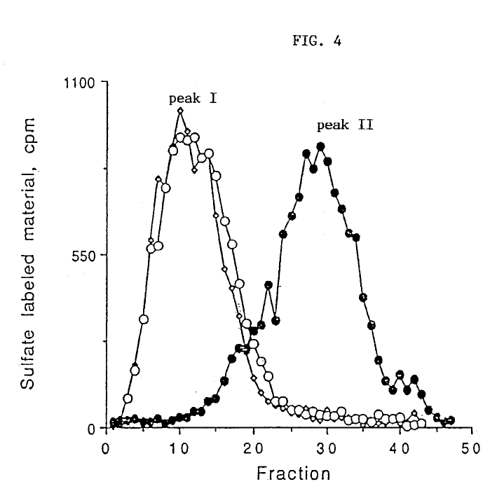

The medium of cells infected with the pFhpa4 virus was passed through a 50 kDa cut off membrane to obtain a crude estimation of the molecular weight of the recombinant heparanase enzyme. As demonstrated in FIG. 4, all the enzymatic activity was retained in the upper compartment and there was no activity in the flow through (<50 kDa) material. This result is consistent with the expected molecular weight of the hpa gene product.

In order to further characterize the hpa product the inhibitory effect of heparin, a potent inhibitor of heparanase mediated HS degradation (40) was examined.

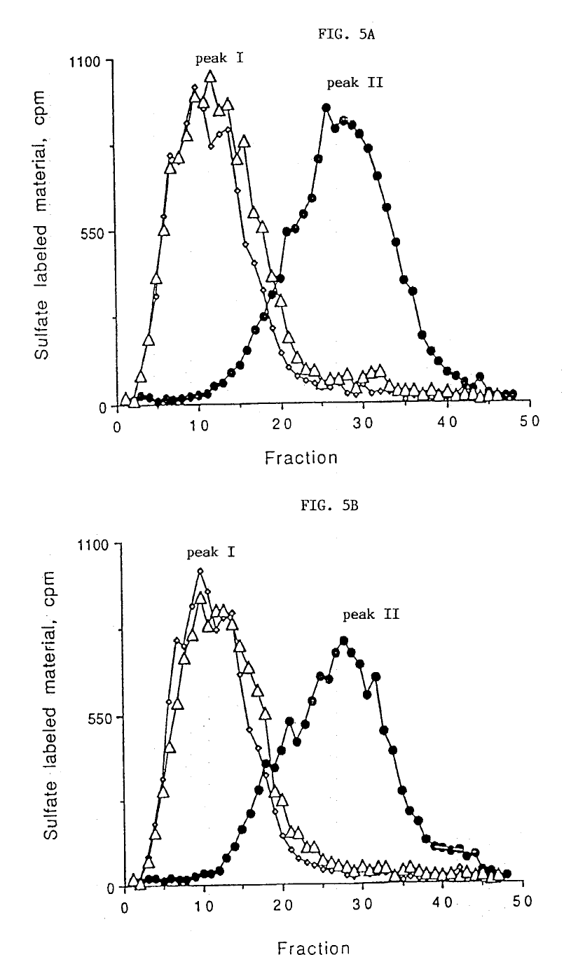

As demonstrated in FIGS. 5a-b, conversion of the peak I substrate into peak II HS degradation fragments was completely abolished in the presence of heparin.

Altogether, these results indicate that the heparanase enzyme is expressed in an active form by insect cells infected with Baculovirus containing the newly identified human hpa gene.

### Example 3

Degradation of HSPG in Intact ECM

Next, the ability of intact infected insect cells to degrade HS in intact, naturally produced ECM was investigated. For this purpose, High Five or Sf21 cells were seeded on metabolically sulfate labeled ECM followed by infection (48 h, 28° C.) with either the pFhpa4 or control pF2 viruses. The pH of the medium was then adjusted to pH 6.2-6.4 and the cells further incubated with the labeled ECM for another 48 h at 28° C. or 24 h at 37° C. Sulfate labeled material released into the incubation medium was analyzed by gel filtration on Sepharose 6B.

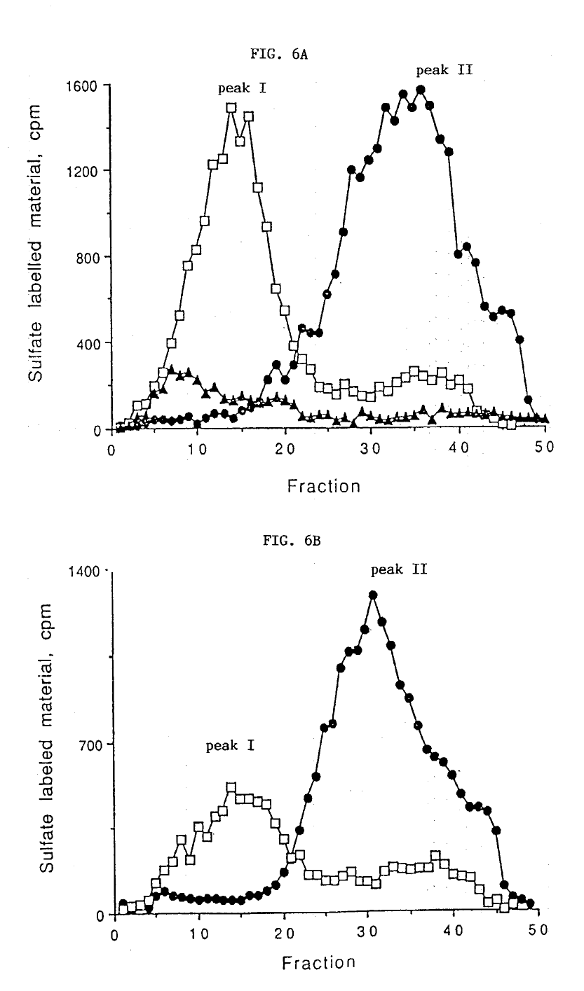

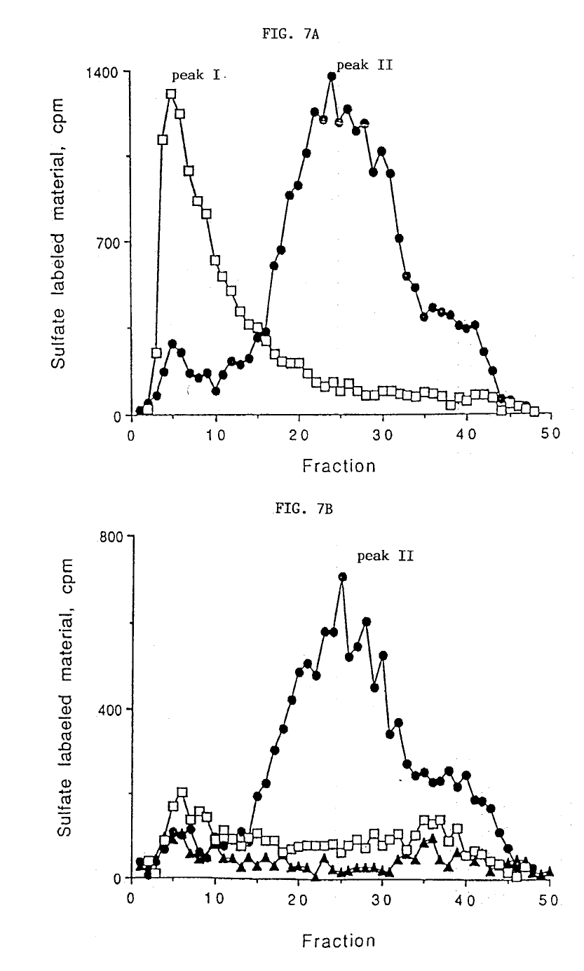

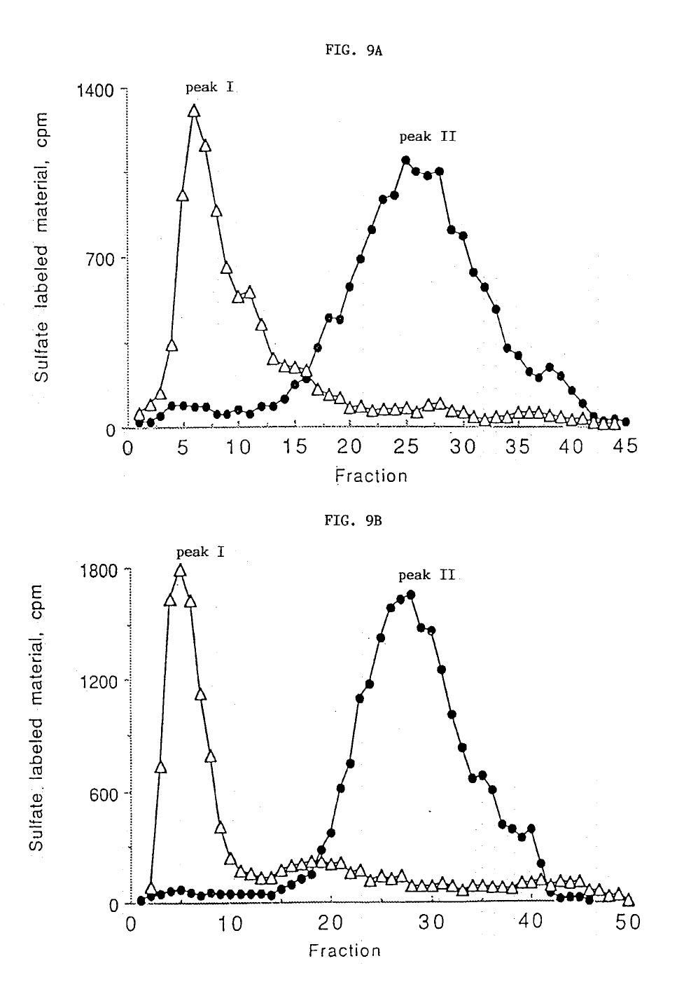

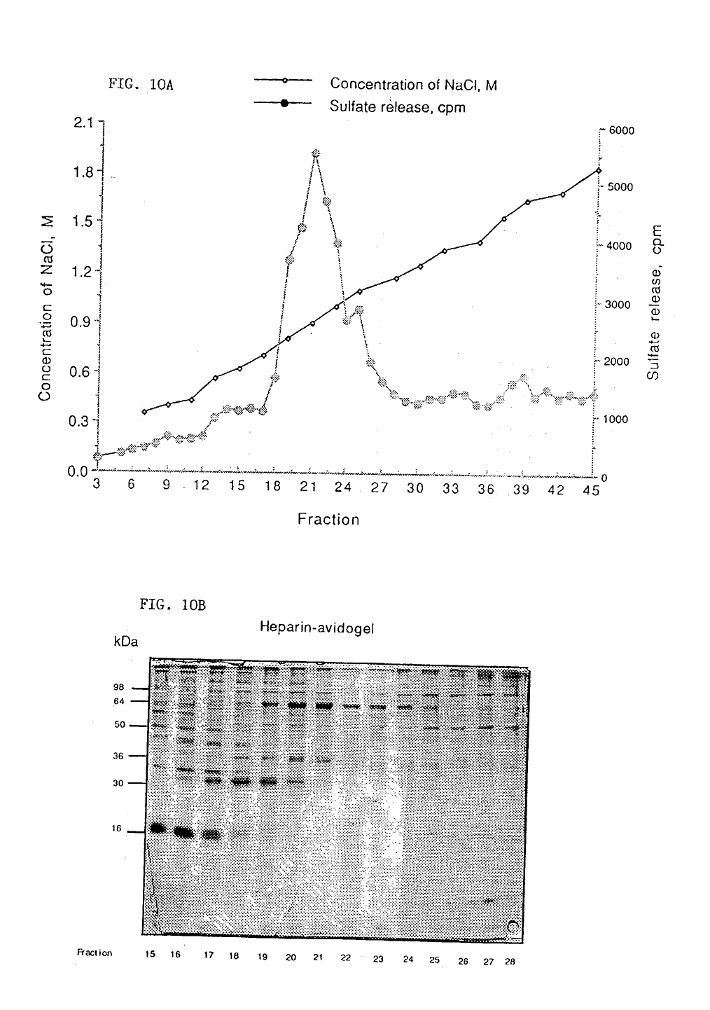

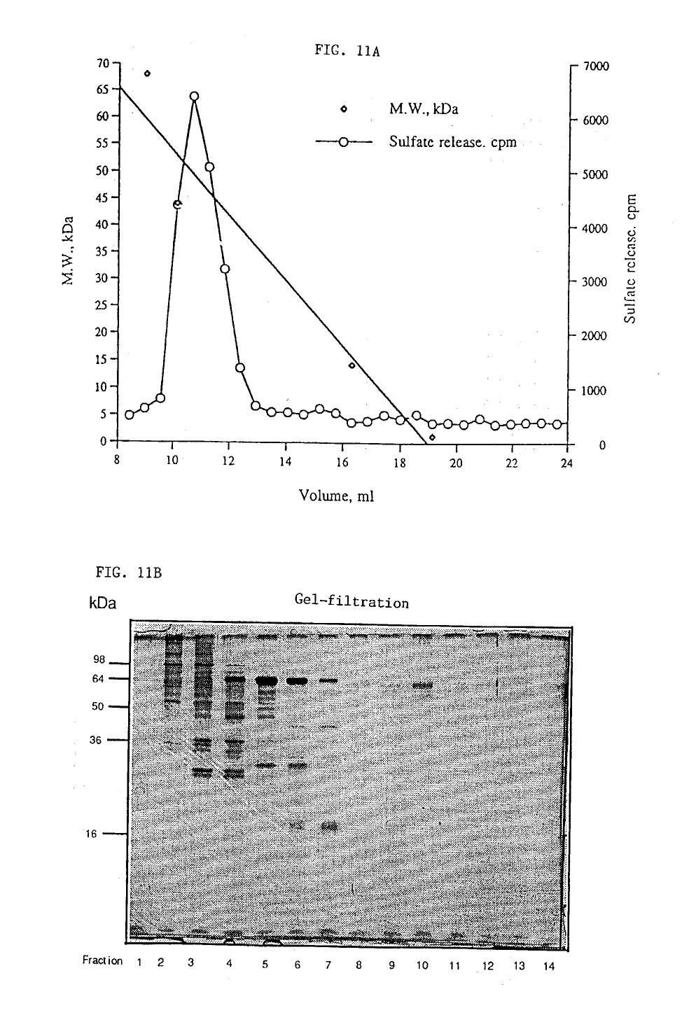

As shown in FIGS. 6a-b and 7a-b, incubation of the ECM with cells infected with the control pF2 virus resulted in a constant release of labeled material that consisted almost entirely (>90%) of high Mr fragments (peak I) eluted with or next to V_{O}. It was previously shown that a proteolytic activity residing in the ECM itself and/or expressed by cells is responsible for release of the high Mr material (6). This nearly intact HSPG provides a soluble substrate for subsequent degradation by heparanase, as also indicated by the relatively large amount of peak I material accumulating when the heparanase enzyme is inhibited by heparin (6, 7, 12, FIG. 9). On the other hand, incubation of the labeled ECM with cells infected with the pFhpa4 virus resulted in release of 60-70% of the ECM-associated radioactivity in the form of low Mr sulfate-labeled fragments (peak II, 0.5<Kav<0.75), regardless of whether the infected cells were incubated with the ECM at 28° C. or 37° C. Control intact non-infected Sf21 or High Five cells failed to degrade the ECM HS side chains.

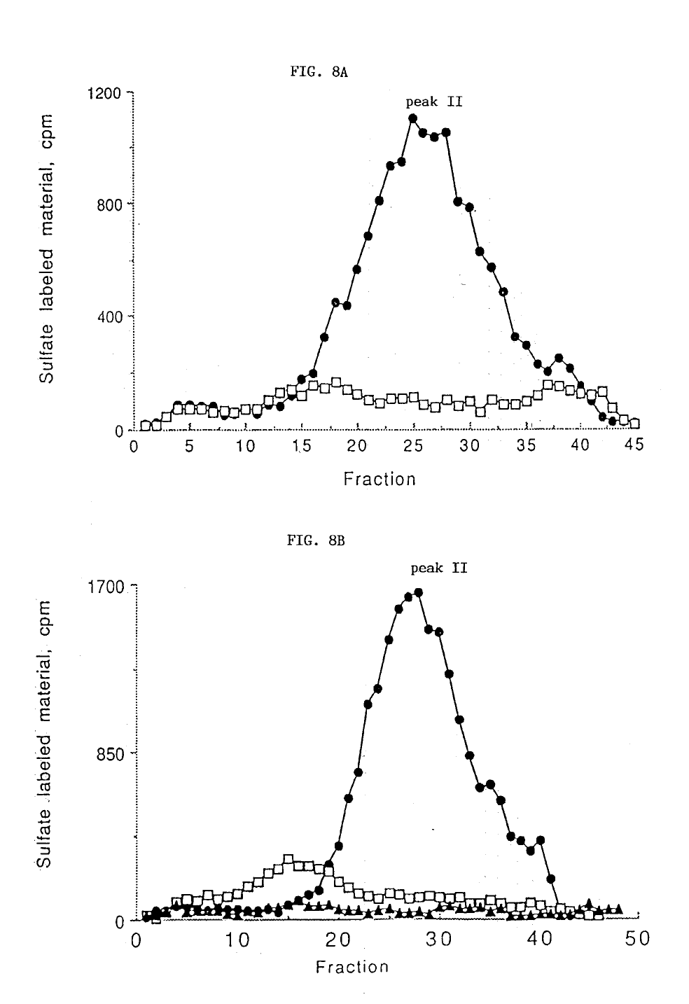

In subsequent experiments, as demonstrated in FIGS. 8a-b, High Five and Sf21 cells were infected (96 h, 28° C.) with pFhpa4 or control pF1 viruses and the culture medium incubated with sulfate-labeled ECM. Low Mr HS degradation fragments were released from the ECM only upon incubation with medium conditioned by pFhpa4 infected cells. As shown in FIG. 9, production of these fragments was abolished in the presence of heparin. No heparanase activity was detected in the culture medium of control, non-infected cells. These results indicate that the heparanase enzyme expressed by cells infected with the pFhpa4 virus is capable of degrading HS when complexed to other macromolecular constituents (i.e. fibronectin, laminin, collagen) of a naturally produced intact ECM, in a manner similar to that reported for highly metastatic tumor cells or activated cells of the immune system (6, 7).

### Example 4

Purification of Recombinant Human Heparanase

The recombinant heparanase was partially purified from medium of pFhpa4 infected Sf21 cells by Heparin-Sepharose chromatography (FIG. 10a) followed by gel filtration of the pooled active fractions over an FPLC Superdex 75 column (FIG. 11a). A˜63 kDa protein was observed, whose quantity, as was detected by silver stained SDS-polyacrylamide gel electrophoresis, correlated with heparanase activity in the relevant column fractions (FIGS. 10b and 11b, respectively). This protein was not detected in the culture medium of cells infected with the control pF1 virus and was subjected to a similar fractionation on heparin-Sepharose (not shown).

### Example 5

Expression of the Human hpa cDNA in Various Cell Types, Organs and Tissues

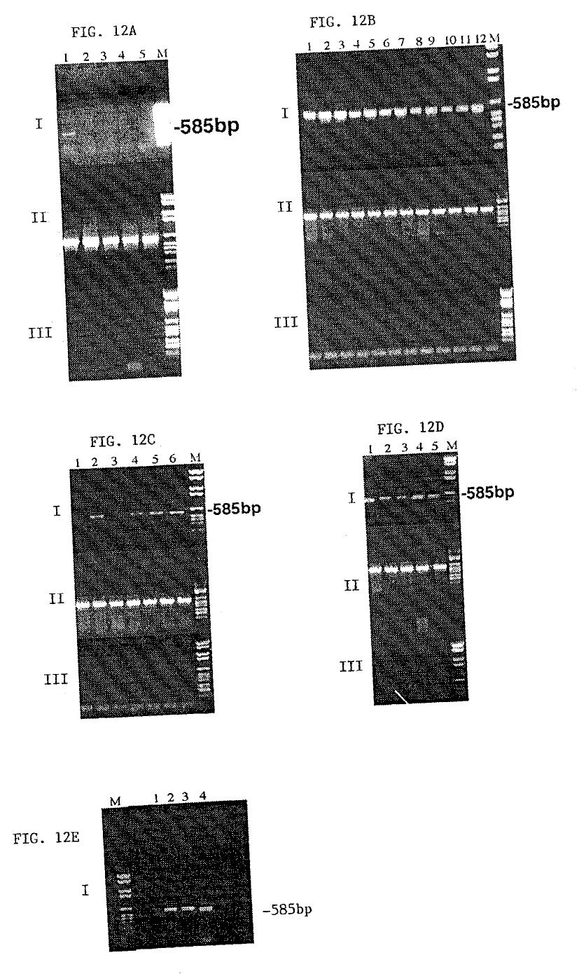

Referring now to FIGS. 12a-e, RT-PCR was applied to evaluate the expression of the hpa gene by various cell types and tissues. For this purpose, total RNA was reverse transcribed and amplified. The expected 585 bp long cDNA was clearly demonstrated in human kidney, placenta (8 and 11 weeks) and mole tissues, as well as in freshly isolated and short termed (1.5-48 h) cultured human placental cytotrophoblastic cells (FIG. 12a), all known to express a high heparanase activity (41). The hpa transcript was also expressed by normal human neutrophils (FIG. 12b). In contrast, there was no detectable expression of the hpa mRNA in embryonic human muscle tissue, thymus, heart and adrenal (FIG. 12b). The hpa gene was expressed by several, but not all, human bladder carcinoma cell lines (FIG. 12c), SK hepatoma (SK-hep-1), ovarian carcinoma (OV 1063), breast carcinoma (435, 231), melanoma and megakaryocytic (DAMI, CHRF) human cell lines (FIGS. 12d-e).

The above described expression pattern of the hpa transcript was determined to be in a very good correlation with heparanase activity levels determined in various tissues and cell types (not shown).

### Example 6

Isolation of an Extended 5′ End of hpa cDNA from Human SK-hep1 Cell Line

The 5′ end of hpa cDNA was isolated from human SK-hep1 cell line by PCR amplification using the Marathon RACE (rapid amplification of cDNA ends) kit (Clontech). Total RNA was prepared from SK-hep1 cells using the TRI-Reagent (Molecular research center Inc.) according to the manufacturer instructions. Poly A+ RNA was isolated using the mRNA separator kit (Clonetech).

The Marahton RACE SK-hep1 cDNA composite was constructed according to the manufacturer recommendations. First round of amplification was performed using an adaptor specific primer AP1: 5′-CCATCCTAATACG ACTCACTATAGGGC-3′, SEQ ID NO:1, and a hpa specific antisense primer hpl-629: 5′-CCCCAGGAGCAGCAGCATCAG-3′, SEQ ID NO:17, corresponding to nucleotides 119-99 of SEQ ID NO:9. The resulting PCR product was subjected to a second round of amplification using an adaptor specific nested primer AP2: 5′-ACTCACTATAGGGCTCGAGCGGC-3′, SEQ ID NO:3, and a hpa specific antisense nested primer hpl-666 5′-AGGCTTCGAGCGCAGCAGCAT-3′, SEQ ID NO:18, corresponding to nucleotides 83-63 of SEQ ID NO:9. The PCR program was as follows: a hot start of 94° C. for 1 minute, followed by 30 cycles of 90° C.-30 seconds, 68° C.-4 minutes. The resulting 300 bp DNA fragment was extracted from an agarose gel and cloned into the vector pGEM-T Easy (Promega). The resulting recombinant plasmid was designated pHPSK1.

The nucleotide sequence of the pHPSK1 insert was determined and it was found to contain 62 nucleotides of the 5′ end of the placenta hpa cDNA (SEQ ID NO:9) and additional 178 nucleotides upstream, the first 178 nucleotides of SEQ ID NOs: 13 and 15.

A single nucleotide discrepancy was identified between the SK-hep1 cDNA and the placenta cDNA. The “T” derivative at position 9 of the placenta cDNA (SEQ ID NO:9), is replaced by a “C” derivative at the corresponding position 187 of the SK-hep1 cDNA (SEQ ID NO:13).

The discrepancy is likely to be due to a mutation at the 5′ end of the placenta cDNA clone as confirmed by sequence analysis of sevsral additional cDNA clones isolated from placenta, which like the SK-hep1 cDNA contained C at position 9 of SEQ ID NO:9.

The 5′ extended sequence of the SK-hep1 hpa cDNA was assembled with the sequence of the hpa cDNA isolated from human placenta (SEQ ID NO:9). The assembled sequence contained an open reading frame which encodes, as shown in SEQ ID NOs:14 and 15, a polypeptide of 592 amino acids with a calculated molecular weight of 66,407 daltons. The open reading frame is flanked by 93 bp 5′ untranslated region (UTR).

### Example 7

Isolation of the Upstream Genomic Region of the hpa Gene

The upstream region of the hpa gene was isolated using the Genome Walker kit (Clontech) according to the manufacturer recommendations. The kit includes five human genomic DNA samples each digested with a different restriction endonuclease creating blunt ends: EcoRV, ScaI, DraI, PvuII and SspI.

The blunt ended DNA fragments are ligated to partially single stranded adaptors. The Genomic DNA samples were subjected to PCR amplification using the adaptor specific primer and a gene specific primer. Amplification was performed with Expand High Fidelity (Boehringer Mannheim).

A first round of amplification was performed using the ap1 primer: 5′-G TAATACGACTCACTATAGGGC-3′, SEQ ID NO:19, and the hpa specific antisense primer hpl-666: 5′-AGGCTTCGAGCGCAGCAGCAT-3′, SEQ ID NO:18, corresponding to nucleotides 83-63 of SEQ ID NO:9. The PCR program was as follows: a hot start of 94° C.-3 minutes, followed by 36 cycles of 94° C.-40 seconds, 67° C.-4 minutes.

The PCR products of the first amplification were diluted 1:50. One μl of the diluted sample was used as a template for a second amplification using a nested adaptor specific primer ap2: 5′-ACTATAGGGCACGCGTGGT-3′, SEQ ID NO:20, and a hpa specific antisense primer hpl-690, 5′-CTTGGGCTCACC TGGCTGCTC-3′, SEQ ID NO:21, corresponding to nucleotides 62-42 of SEQ ID NO:9. The resulting amplification products were analyzed using agarose gel electrophoresis. Five different PCR products were obtained from the five amplification reactions. A DNA fragment of approximately 756 bp which was obtained from the SspI digested DNA sample was gel extracted. The purified fragment was ligated into the plasmid vector pGEM-T Easy (Promega). The resulting recombinant plasmid was designated pGHP6905 and the nucleotide sequence of the hpa insert was determined.

A partial sequence of 594 nucleotides is shown in SEQ ID NO:16. The last nucleotide in SEQ ID NO:13 corresponds to nucleotide 93 in SEQ ID:13. The DNA sequence in SEQ ID NO:16 contains the 5′ region of the hpa cDNA and 501 nucleotides of the genomic upstream region which are predicted to contain the promoter region of the hpa gene.

### Example 8

Expression of the 592 Amino Acids HPA Polypeptide in a Human 293 Cell Line

The 592 amino acids open reading frame (SEQ ID NOs:13 and 15) was constructed by ligation of the 110 bp corresponding to the 5′ end of the SK-hep1 hpa cDNA with the placenta cDNA. More specifically the Marathon RACE-PCR amplification product of the placenta hpa DNA was digested with SacI and an approximately 1 kb fragment was ligated into a SacI-digested pGHP6905 plasmid. The resulting plasmid was digested with EarI and AatII. The EarI sticky ends were blunted and an approximately 280 bp EarI/blunt-AatII fragment was isolated. This fragment was ligated with pFasthpa digested with EcoRI which was blunt ended using Klenow fragment and further digested with AatII. The resulting plasmid contained a 1827 bp insert which includes an open reading frame of 1776 bp, 31 bp of 3′ UTR and 21 bp of 5′ UTR. This plasmid was designated pFastLhpa.

A mammalian expression vector was constructed to drive the expression of the 592 amino acids heparanase polypeptide in human cells. The hpa cDNA was excised prom pFastLhpa with BssHII and NotI. The resulting 1850 bp BssHII-NotI fragment was ligated to a mammalian expression vector pSI (Promega) digested with MluI and NotI. The resulting recombinant plasmid, pSIhpaMet2 was transfected into a human 293 embryonic kidney cell line.

Transient expression of the 592 amino-acids heparanase was examined by western blot analysis and the enzymatic activity was tested using the gel shift assay. Both these procedures are described in length in U.S. patent application Ser. No. 09/071,739, filed May 1, 1998, which is incorporated by reference as if fully set forth herein. Cells were harvested 3 days following transfection. Harvested cells were re-suspended in lysis buffer containing 150 mM NaCl, 50 mM Tris pH 7.5, 1% Triton X-100, 1 mM PMSF and protease inhibitor cocktail (Boehringer Mannheim). 40 μg protein extract samples were used for separation on a SDS-PAGE. Proteins were transferred onto a PVDF Hybond-P membrane (Amersham). The membrane was incubated with an affinity purified polyclonal anti heparanase antibody, as described in U.S. patent application Ser. No. 09/071,739. A major band of approximately 50 kDa was observed in the transfected cells as well as a minor band of approximately 65 kDa. A similar pattern was observed in extracts of cells transfected with the pShpa as demonstrated in U.S. patent application Ser. No. 09/071,739. These two bands probably represent two forms of the recombinant heparanase protein produced by the transfected cells. The 65 kDa protein probably represents a heparanase precursor, while the 50 kDa protein is suggested herein to be the processed or mature form.

The catalytic activity of the recombinant protein expressed in the pShpaMet2 transfected cells was tested by gel shift assay. Cell extracts of transfected and of mock transfected cells were incubated overnight with heparin (6 μg in each reaction) at 37° C., in the presence of 20 mM phosphate citrate buffer pH 5.4, 1 mM CaCl₂, 1 mM DTT and 50 mM NaCl. Reaction mixtures were then separated on a 10% polyacrylamide gel. The catalytic activity of the recombinant heparanase was clearly demonstrated by a faster migration of the heparin molecules incubated with the transfected cell extract as compared to the control. Faster migration indicates the disappearance of high molecular weight heparin molecules and the generation of low molecular weight degradation products.

### Example 9

Chromosomal Localization of the hpa Gene

Chromosomal mapping of the hpa gene was performed utilizing a panel of monochromosomal human/CHO and human/mouse somatic cell hybrids, obtained from the UK HGMP Resource Center (Cambridge, England).

40 ng of each of the somatic cell hybrid DNA samples were subjected to PCR amplification using the hpa primers: hpu565 5′-AGCTCTGTAGATGTGC TATACAC-3′, SEQ ID NO:22, corresponding to nucleotides 564-586 of SEQ ID NO:9 and an antisense primer hpl171 5′-GCATCTTAGCCGTCTTTCTTCG-3′, SEQ ID NO:23, corresponding to nucleotides 897-876 of SEQ ID NO:9.

The PCR program was as follows: a hot start of 94° C.-3 minutes, followed by 7 cycles of 94° C.-45 seconds, 66° C.-1 minute, 68° C.-5 minutes, followed by 30 cycles of 94° C.-45 seconds, 62° C.-1 minute, 68° C.-5 minutes, and a 10 minutes final extension at 72° C.

The reactions were performed with Expand long PCR (Boehringer Mannheim). The resulting amplification products were analyzed using agarose gel electrophoresis. As demonstrated in FIG. 14, a single band of approximately 2.8 Kb was obtained from chromosome 4, as well as from the control human genomic DNA. A 2.8 kb amplification product is expected based on amplification of the genomic hpa clone (data not shown). No amplification products were obtained neither in the control DNA samples of hamster and mouse nor in somatic hybrids of other human chromosome.

### Example 10

Human Genomic Clone Encoding Heparanase

Five plaques were isolated following screening of a human genomic library and were designated L3-1, L5-1, L8-1, L10-1 and L6-1. The phage DNAs were analyzed by Southern hybridization and by PCR with hpa specific and vector specific primers. Southern analysis was performed with three fragments of hpa cDNA: a PvuII-BamHI fragment (nucleotides 32-450, SEQ ID NO:9), a BamHI-NdeI fragment (nucleotides 451-1102, SEQ ID NO:9) and an NdeI-XhoI fragment (nucleotides 1103-1721, SEQ ID NO:9).

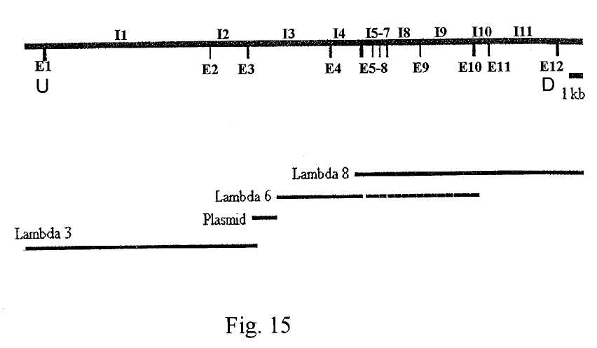

Following Southern analysis, phages L3, L6, L8 were selected for further analysis. A scheme of the genomic region and the relative position of the three phage clones is depicted in FIG. 15. A 2 kb DNA fragment containing the gap between phages L6 and L3 was PCR amplified from human genomic DNA with two gene specific primers GHpuL3 and GHplL6. The PCR product was cloned into the plasmid vector pGEM-T-easy (Promega).

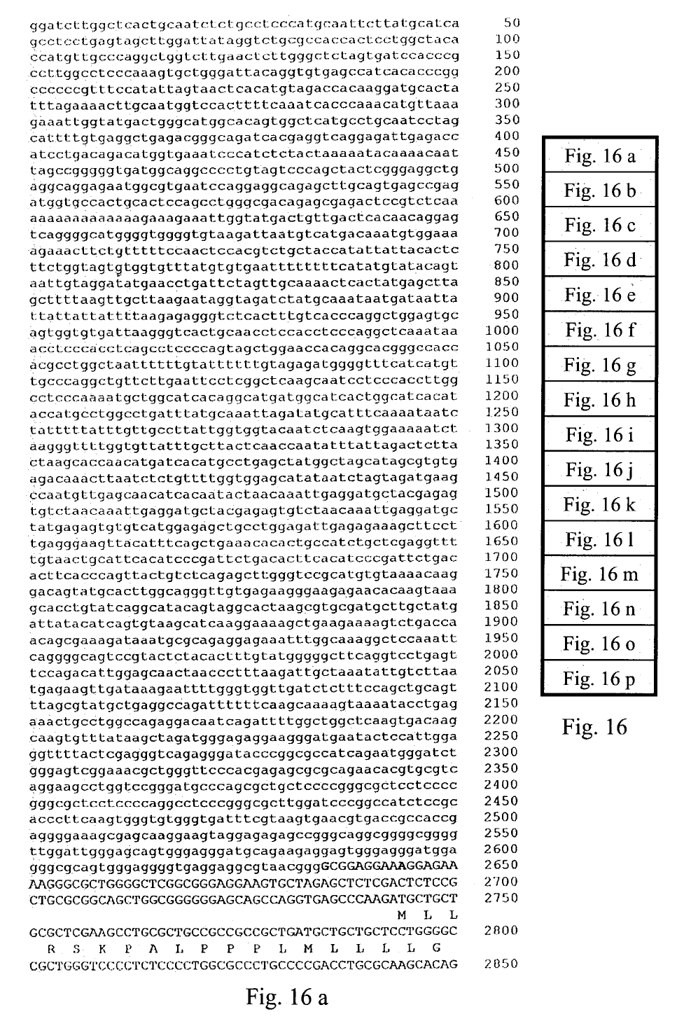

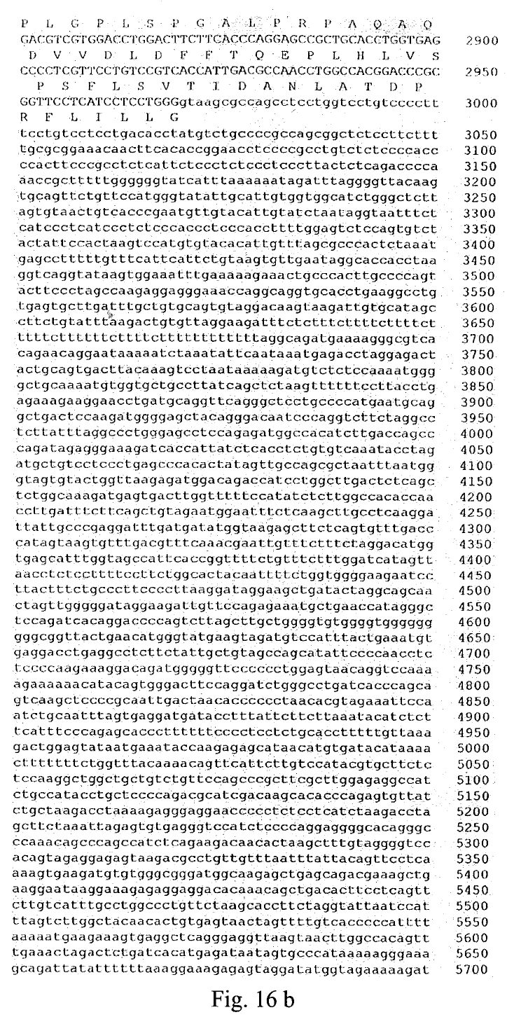

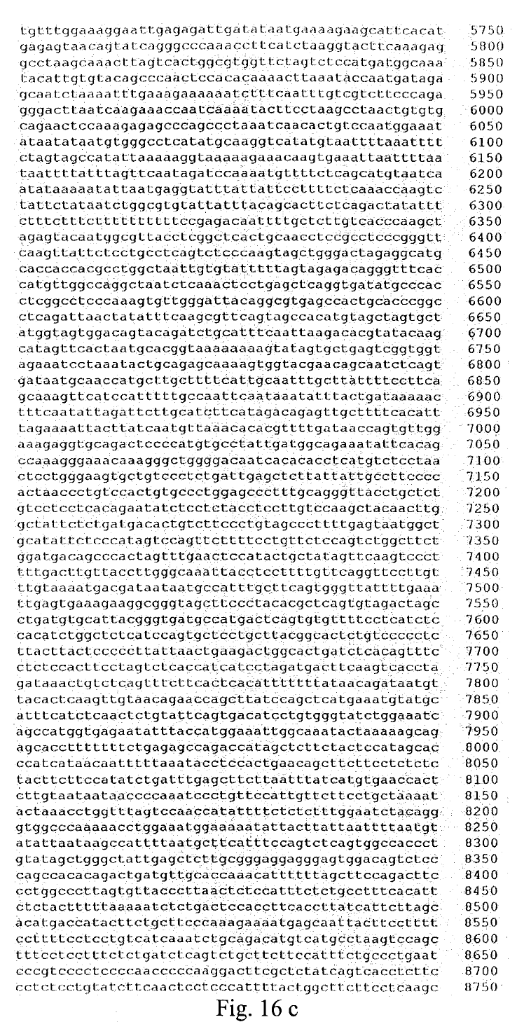

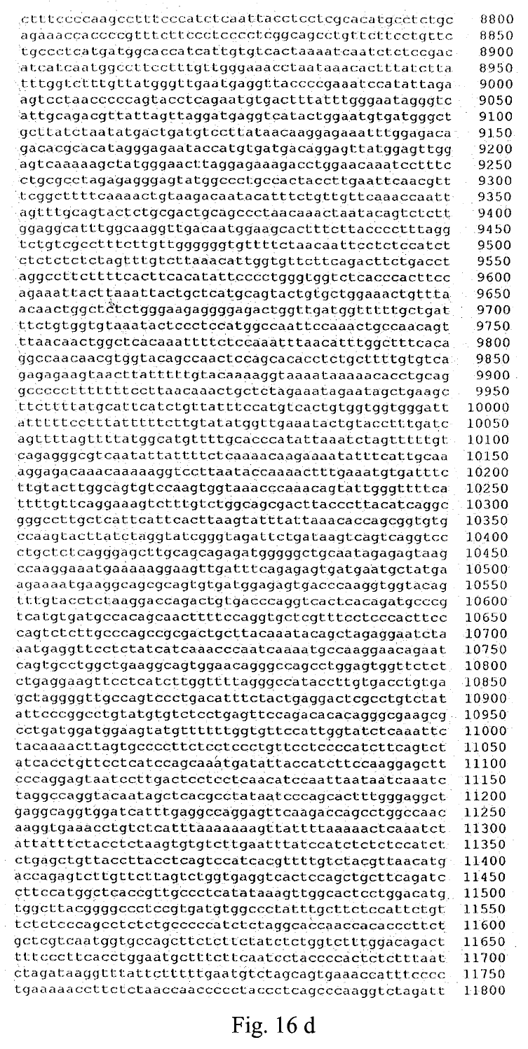

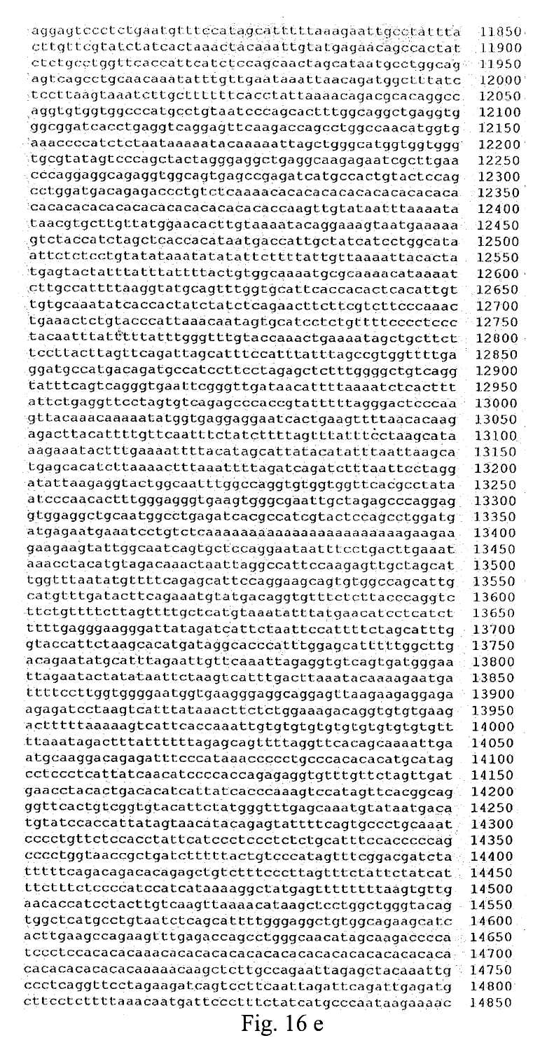

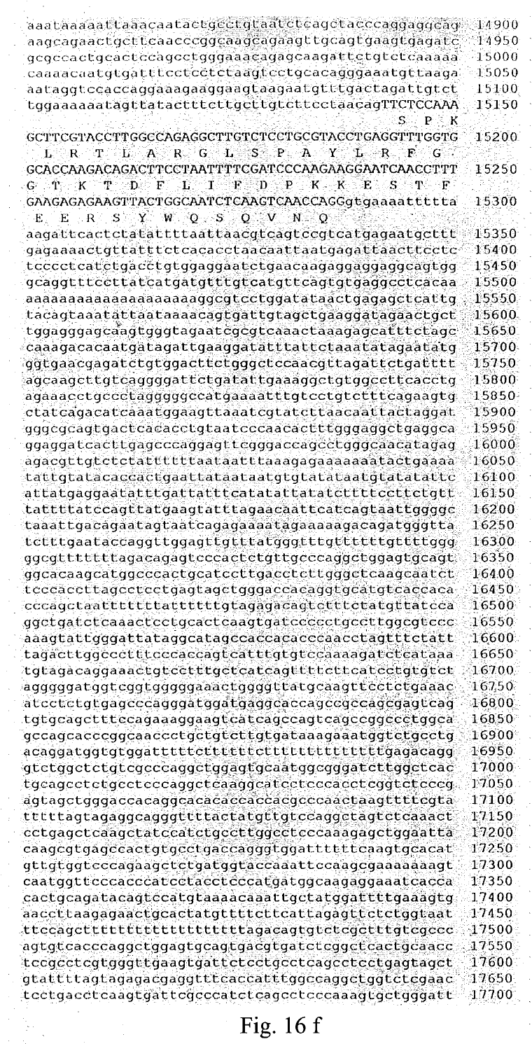

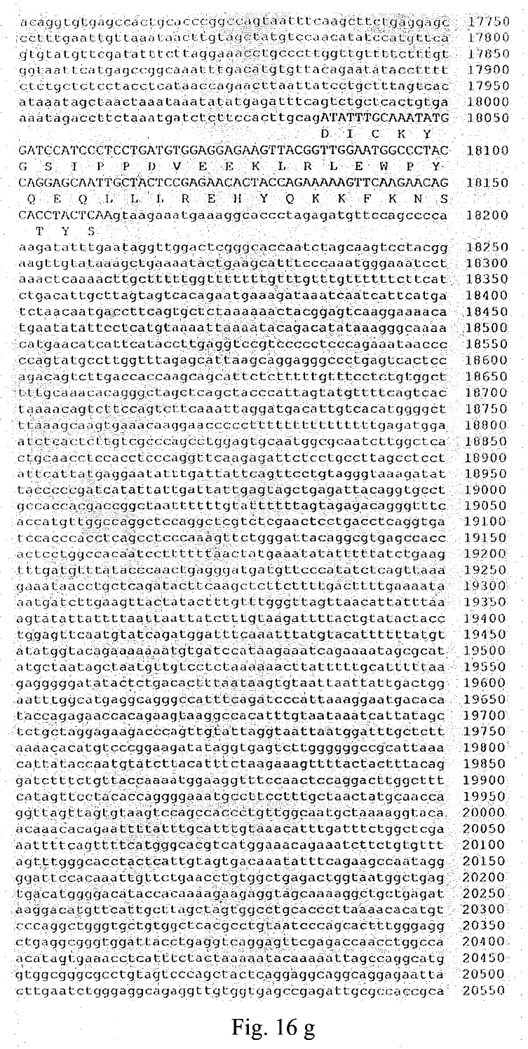

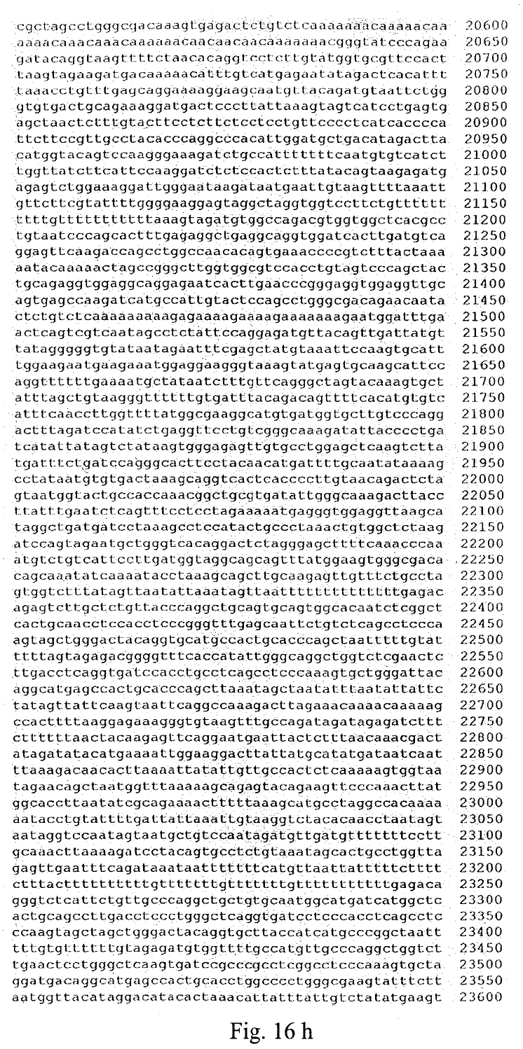

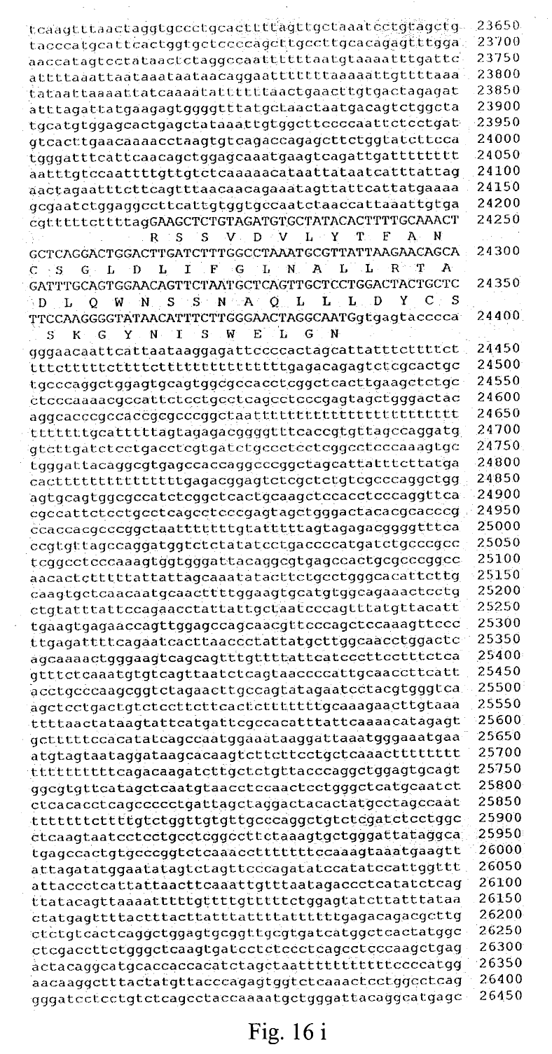

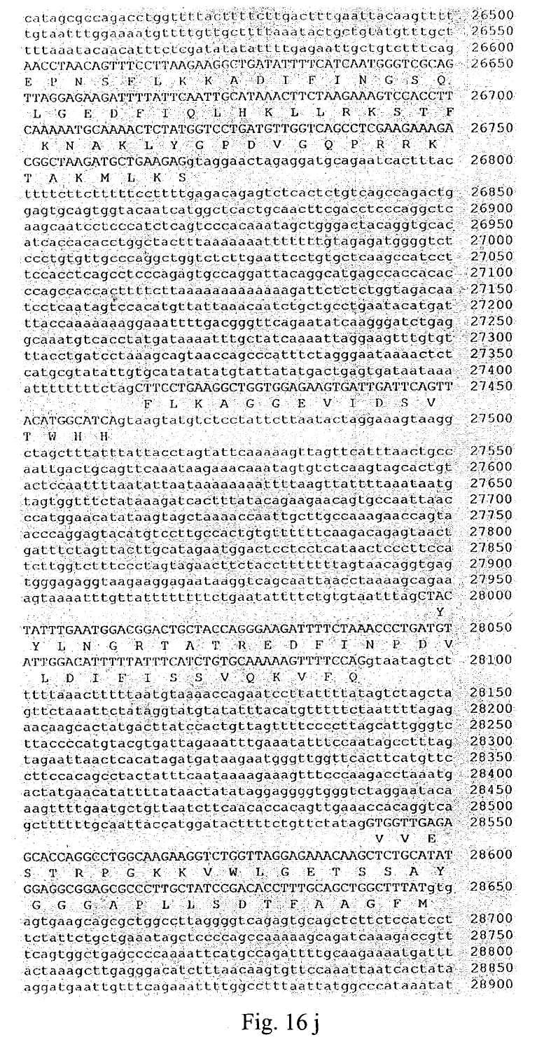

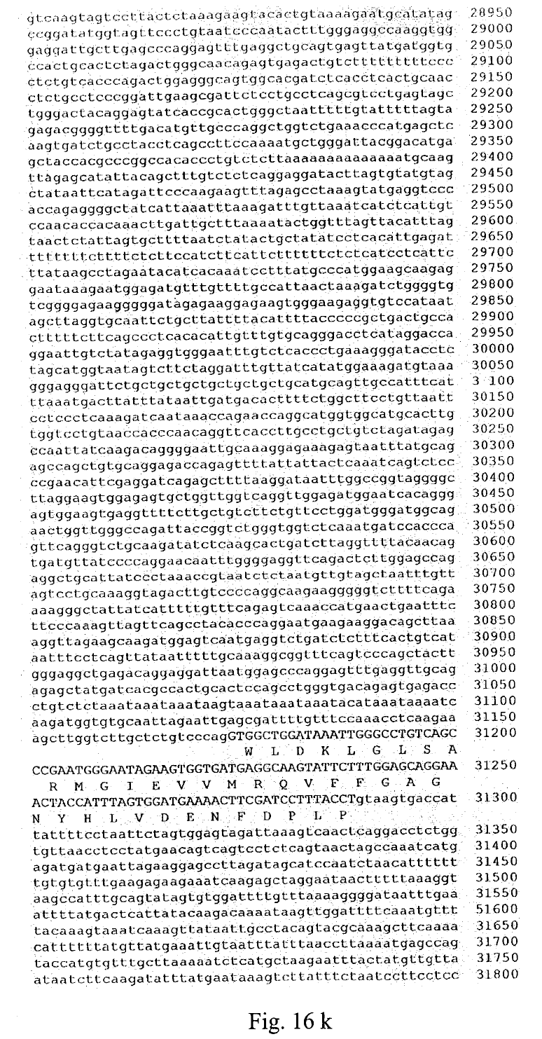

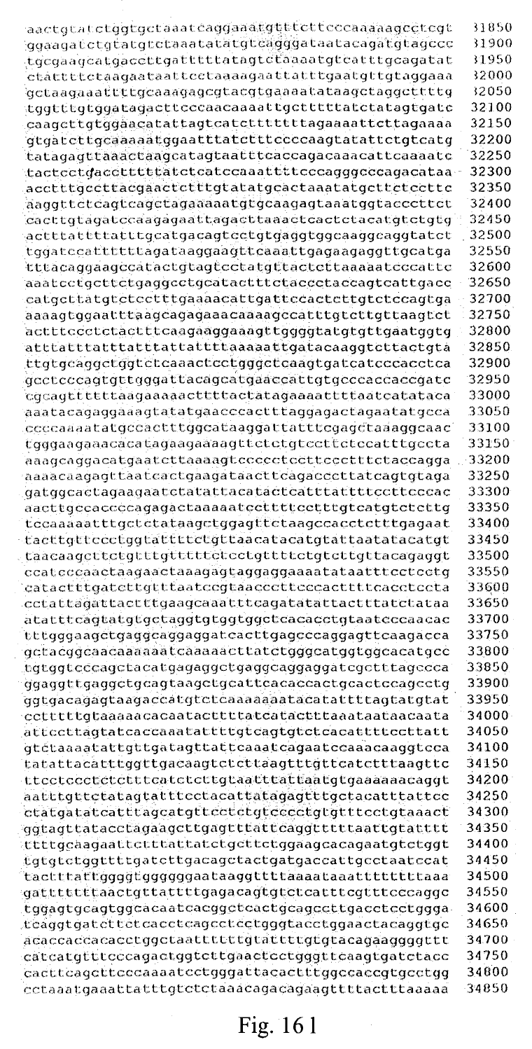

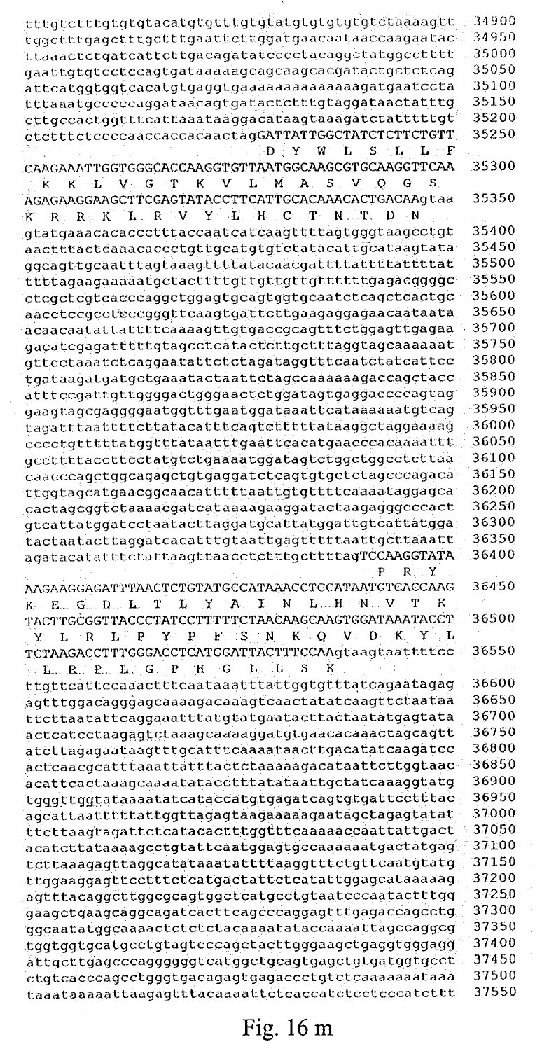

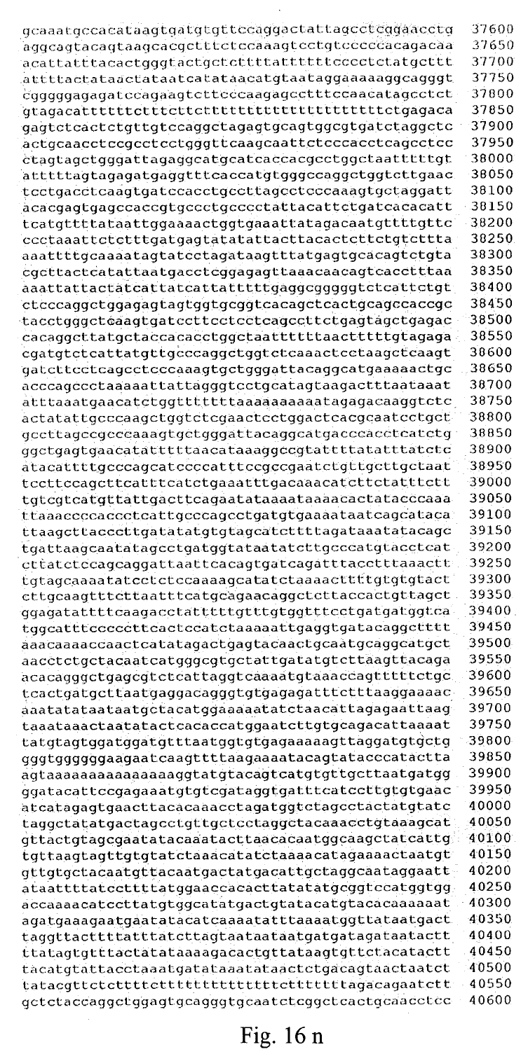

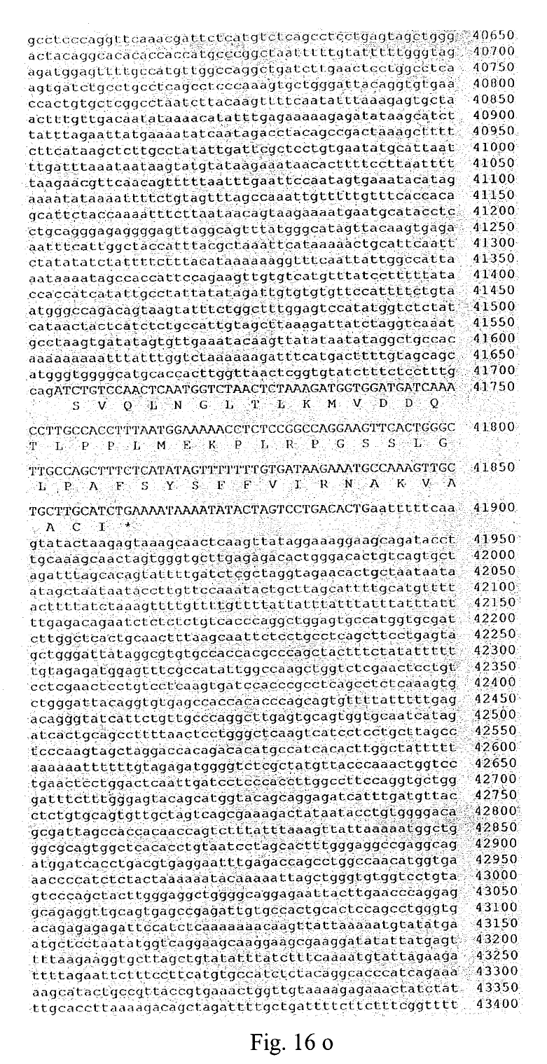

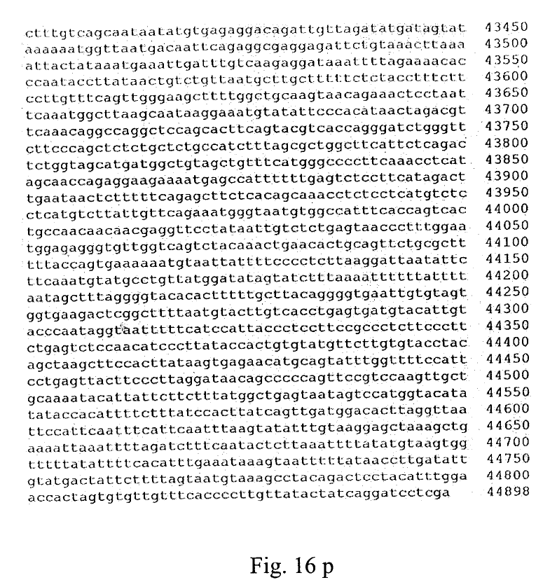

Large scale DNA sequencing of the three Lambda clones and the amplified fragment was performed with Lambda purified DNA by primer walking. A nucleotide sequence of 44,898 bp was analyzed (FIG. 16, SEQ ID NO:42). Comparison of the genomic sequence with that of hpa cDNA revealed 12 exons separated by 11 introns (FIGS. 15 an 16). The genomic organization of the hpa gene is depicted in FIG. 15 (top). The sequence include the coding region from the first ATG to the stop codon which spans 39,113 nucleotides, 2742 nucleotides upstream of the first ATG and 3043 nucleotides downstream of the stop codon. Splice site consensus sequences were identified at exon/intron junctions.

### Example 11

Alternative Splicing

Several minor RT-PCR products were obtained from various cell types, following amplification with hpa specific primers. Each one found to contain a deletion of one or two exons. Some of these PCR products contain ORFs, which encode potential shorter proteins.

Table 1 below summarizes the alternative spliced products isolated from various cell lines.

Fragments of similar sizes were obtained following amplification with two cell lines, placenta and platelets.

| Cell type | Nucleotides deleted | Exons deleted | ORF |
|---|---|---|---|
| Platelets | 1047-1267 | 8, 9 | + |
| Platelets | 1154-1267 | 9 | − |
| Platelets | 289-435, 562-735 | 2, 4 | − |
| Sk-hep1, platelets, Zr75 | 562-735 | 4 | + |
| Sk-hep1 (hepatoma) | 561-904 | 4, 5 | − |
| Zr75 (breast carcinoma) | 96-203 | 1 (partial) | + |

### Example 12

Mouse and Rat hpa

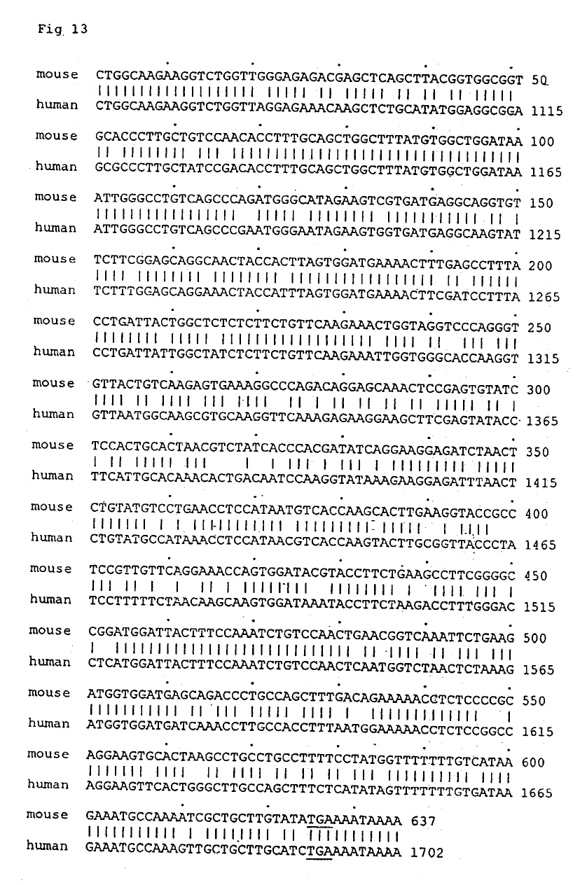

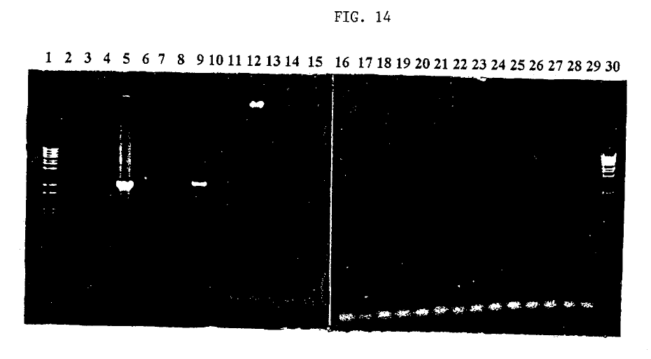

EST databases were screened for sequences homologous to the hpa gene. Three mouse EST's were identified (accession No. Aa177901, from mouse spleen, Aa067997 from mouse skin, Aa47943 from mouse embryo), assembled into a 824 bp cDNA fragment which contains a partial open reading frame (lacking a 5′ end) of 629 bp and a 3′ untranslated region of 195 bp (SEQ ID NO:12). As shown in FIG. 13, the coding region is 80% similar to the 3′ end of the hpa cDNA sequence. These EST's are probably cDNA fragments of the mouse hpa homolog that encodes for the mouse heparanase.

Searching for consensus protein domains revealed an amino terminal homology between the heparanase and several precursor proteins such as Procollagen Alpha 1 precursor, Tyrosine-protein kinase-RYK, Fibulin-1, Insulin-like growth factor binding protein and several others. The amino terminus is highly hydrophobic and contains a potential trans-membrane domain. The homology to known signal peptide sequences suggests that it could function as a signal peptide for protein localization.

The amino acid sequence of human heparanase was used to search for homologous sequences in the DNA and protein databases. Several human EST's were identified, as well as mouse sequences highly homologous to human heparanase. The following mouse EST's were identified AA177901, AA674378, AA67997, AA047943, AA690179, AI122034, all sharing an identical sequence and correspond to amino acids 336-543 of the human heparanase sequence. The entire mouse heparanase cDNA was cloned, based on the nucleotide sequence of the mouse EST's. PCR primers were designed and a Marathon RACE was performed using a Marathon cDNA library from 15 days mouse embryo (Clontech) and from BL6 mouse melanoma cell line. The mouse hpa homologous cDNA was isolated following several amplification steps. A 1.1 kb fragment was amplified from mouse embryo Marathon cDNA library. The first cycle of amplification was performed with primers mhpl773 and Ap1 and the second cycle with primers mhpl736 and AP2. A 1.1 kb fragment was then amplified from BL6 Marathon cDNA library. The first cycle of amplification was performed with the primers mhpl152 and Ap1, and the second with mhpl83 and AP2. The combined sequence was homologous to nucleotides 157-1702 of the human hpa cDNA, which encode amino acids 33-543. The 5′ end of the mouse hpa gene was isolated from a mouse genomic DNA library using the Genome Walker kit (Clontech). An 0.9 kb fragment was amplified from a DraI digested Genome walker DNA library. The first cycle of amplification was performed with primers mhpl114 and Ap1 and the second with primers mhpl103 and AP2. The assembled sequence (SEQ ID NOs:43, 45) is 2396 nucleotides long. It contains an open reading frame of 1605 nucleotides, which encode a polypeptide of 535 amino acids (SEQ ID NOs:44, 45), 196 nucleotides of 3′ untranslated region (UTR), and an upstream sequence which includes the promoter region and the 5′-UTR of the mouse hpa cDNA. According to two promoter predicting programs TSSW and TSSG, the transcription start site is localized to nucleotide 431 of SEQ ID NOs:43, 45, 163 nucleotides upstream of the first ATG codon. The 431 upstream genomic sequence contains the promoter region. A TATA box is predicted at position 394 of SEQ ID NOs:43, 45. The mouse and the human hpa genes share an average homology of 78% between the nucleotide sequences and 81% similarity between the deduced amino acid sequences.

Search for hpa homologous sequences, using the Blast 2.0 server revealed two EST's from rat: AI060284 (385 nucleotides, SEQ ID NO:46) which is homologous to the amino terminus (68% similarity to amino acids 12-136) of human heparanase and AI237828 (541 nucleotides, SEQ ID NO:47) which is homologous to the carboxyl terminus (81% similarity to amino acids 500-543) of human heparanase, and contains a 3′-UTR. A comparison between the human heparanase and the mouse and rat homologous sequences is demonstrated in FIG. 17.

### Example 13

Prediction of Heparanase Active Site

Homology search of heparanase amino acid sequence against the DNA and the protein databases revealed no significant homologies. The protein secondary structure as predicted by the PHD program consists of alternating alpha helices and beta sheets. The fold recognition server of UCLA predicted alpha/beta barrel structure, with under-threshold confidence.

Five of 15 proteins, which were predicted to have most similar folds, were glycosyl hydrolases from various organisms: 1xyza—xylanase from Clostridium Thermocellum, 1pbga—6-phospho-beta-δ-galactosidase from Lactococcus Lactis, 1amy—alpha-amylase from Barley, 1ecea—endocellulase from Acidothermus Cellulolyticus and 1qbc—hexosaminidase alpha chain, glycosyl hydrolase.

Protein homology search using the bioaccelerator pulled out several proteins, including glycosyl hydrolyses such as beta-fructofuranosidase from Vicia faba (broad bean) and from potato, lactase phlorizin hydrolase from human, xylanases from Clostridium thermocellum and from Streptomyces halstedii and cellulase from Clostridium thermocellum. Blocks 9.3 database pulled out the active site of glycosyl hydrolases family five, which includes cellulases from various bacteria and fungi. Similar active site motif is shared by several lysosomal acid hydrolases (63) and other glycosyl hydrolases. The common mechanism shared by these enzymes involves two glutamic acid residues, a proton donor and a nucleophile.

Despite the lack of an overall homology between the heparanase and other glycosyl hydolases, the amino acid couple Asp-Glu (NE), which is characteristic of the proton donor of glycosyl hydrolyses of the GH-A clan, was found at positions 224-225 of the human heparanase protein sequence. As in other clan members, this NE couple is located at the end of a β sheet.

Considering the relative location of the proton donor and the predicted secondary structure, the glutamic acid that functions as nucleophile is most likely located at position 343, or at positon 396. Identification of the active site and the amino acids directly involved in hydrolysis opens the way for expression of the defined catalytic domain. In addition, it will provide the tools for rational design of enzyme activity either by modification of the microenviroment or catalytic site itself

### Example 14

Expression of hpa Antisense in Mammalian Cell Lines

A mammalian expression vector Hpa2Kepcdna3 was constructed in order to express hpa antisense in mammalian cells. hpa cDNA (1.7 kb EcoRI fragment) was cloned into the plasmid pCDNA3 in 3′>5′ (antisense) orientation. The construct was used to transfect MBT2-T50 and T24P cell lines. 2×10⁵ cells in 35 mm plates were transfected using the Fugene protocol (Boehringer Mannheim). 48 hours after transfection cells were trypsinized and seeded in six well plates. 24 hours later G418 was added to initiate selection. The number of colonies per 35 mm plate following 3 weeks:

| | Antisense | No insert |
|---|---|---|
| T24P | 15 | 60 |
| MBT-T50 | 1 | 6 |

The lower number of colonies obtained after transfection with hpa antisense, as compared with the control plasmid suggests that the introduction of hpa antisense interfere with cell growth. This experiment demonstrates the use of complementary antisense hpa DNA sequence to control heparanase expression in cells. This approach may be used to inhibit expression of heparanase in vivo, in, for example, cancer cells and in other pathological processes in which heparanase is involved.

### Example 15

Zoo Blot

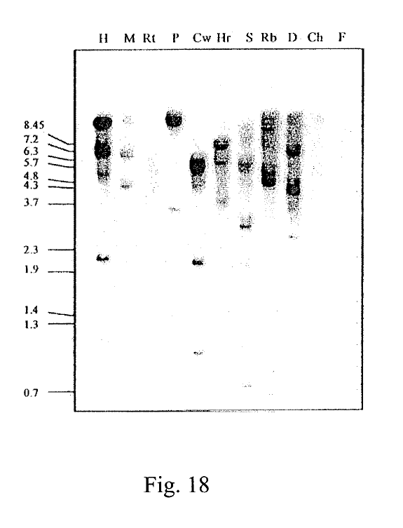

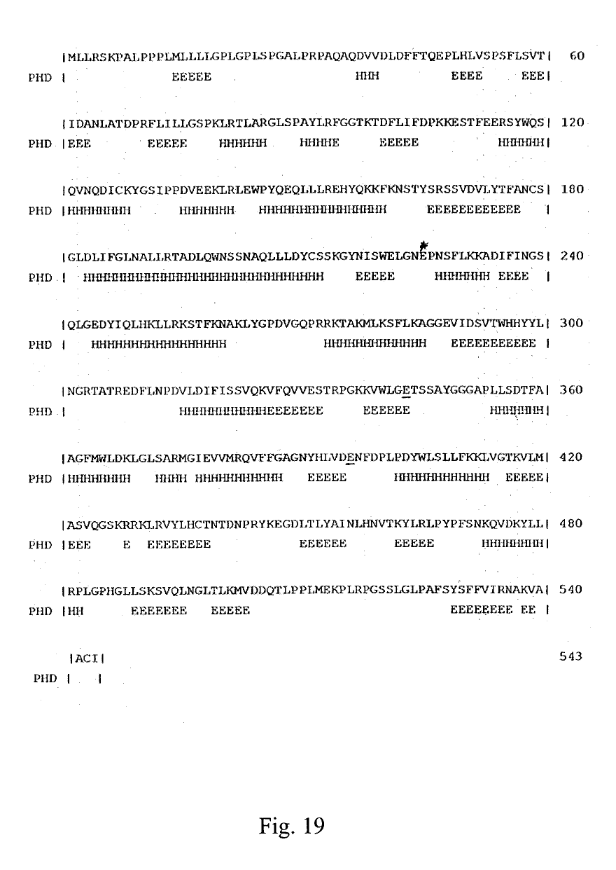

Hpa cDNA was used as a probe to detect homologous sequences in human DNA and in DNA of various animals. The autoradiogram of the Southern analysis is presented in FIG. 18. Several bands were detected in human DNA, which correlated with the accepted pattern according to the genomic hpa sequence. Several intense bands were detected in all mammals, while faint bands were detected in chicken. This correlates with the phylogenetic relation between human and the tested animals. The intense bands indicate that hpa is conserved among mammals as well as in more genetically distant organisms. The multiple bands patterns suggest that in all animals, like in human, the hpa locus occupy large genomic region. Alternatively, the various bands could represent homologous sequences and suggest the existence of a gene family, which can be isolated based on their homology to the human hpa reported herein. This conservation was actually found, between the isolated human hpa cDNA and the mouse homologue.

### Example 16

Characterization of the hpa Promoter

The DNA sequence upstream of the hpa first ATG was subjected to computational analysis in order to localize the predicted transcription start site and to identify potential transcription factors binding sites. Recognition of human PolII promoter region and start of transcription were predicted using the TSSW and TSSG programs. Both programs identified a promoter region upstream of the coding region. TSSW pointed at nucleotide 2644 and TSSG at 2635 of SEQ ID NO:42. These two predicted transcription start sites are located 4 and 13 nucleotides upstream of the longest hpa cDNA isolated by RACE.

A hpa promoter-GFP reporter vector was constructed in order to investigate the regulation of hpa transcription. Two constructs were made, containing 1.8 kb and 1.1 kb of the hpa promoter region. The reporter vector was transfected into T50-mouse bladder carcinoma cells. Cells transfected with both constructs exhibited green fluorescence, which indicated the promoter activity of the genomic sequence upstream of the hpa-coding region. This reporter vector, enables the monitoring of hpa promoter activity, at various conditions and in different cell types and to characterize the factors involved regulation of hpa expression.

### Example 17

Inhibition of Heparanase Expression by Antisense Oligonucleotides

Antisense oligonucleotides (and/or nucleic acid constructs) which are encompassed by the present invention include, but are not limited to, all oligonucleotides of at least 10 bases which are capable of hybridizing in vivo, under physiological conditions, with a portion of a polynucleotide strand encoding a polypeptide having heparanase catalytic activity, as well as any sequence from which such an antisense oligonucleotide may be obtained, for example through synthesis. For example, preferably a nucleic acid construct from which such an antisense oligonucleotide may be obtained includes and/or features a promoter sequence and a polynucleotide sequence directing the synthesis of an antisense RNA oligonucleotide having the previously described characteristics of the antisense oligonucleotide.

This Example describes data obtained with the antisense oligonucleotides of the present invention, as well as different embodiments of these oligonucleotides, and pharmaceutical compositions and uses/methods of treatment thereof.

Experimental Methods and Materials

A stable CHO clone T1-1, transfected with human heparanase cDNA (as described in U.S. Pat. Nos. 6,475,763, 6,426,209 and 6,348,344: Genetically modified cells and methods for expressing recombinant heparanase and methods of purifying same, all of which are hereby incorporated by reference as if fully set forth herein) and expressing active human heparanase protein was treated with antisense oligonucleotides. Inhibitory effect of the antisense molecules was demonstrated as a decrease in heparanase levels in extracts of antisense treated cells.

Chimeric antisense oligonucleotides (Microsynth) were designed as 2′-O-methyl RNA “wings” (5 nucleotides on each end) with a phosphorothioate DNA center (10 nucleotides) (Anissa et al. Nucleic Acid Research 200-1, vol. 29, No. 8 1683-1689, also incorporated by reference as if fully set forth herein). The antisense oligonucleotides were designed to target various sites of the heparanase cDNA as follows (the position on SEQ ID NO 9, heparanase cDNA nucleotide sequence is indicated on the right of each oligonucleotide):

(SEQ ID NO:48)

| | |
|---|---|
| as 175: | GmAmAmGmAmA*G*T*C*C*A*G*G*T*C*CmAmCmGmAm |
| (194-175) | |

(SEQ ID NO:49)

| | |
|---|---|
| as 312: | GmCmAmGmGmA*G*A*C*A*A*G*C*C*T*CmUmGmGmCm |
| (331-312) | |

(SEQ ID NO:50)

| | |
|---|---|
| as 511: | GmUmAmGmUmG*T*T*C*T*C*G*G*A*G*UmAmGmCmAm |
| (530-511) | |

(SEQ ID NO:51)

| | |
|---|---|
| as 681: | GmAmAmGmAmG*C*A*G*T*A*G*T*C*C*AmGmGmAmGm |
| (700-681) | |

(SEQ ID NO:52)

| | |
|---|---|
| as 817: | GmAmAmGmGmT*G*G*A*C*T*T*T*C*T*UmAmGmAmAm |
| (836-817) | |

(SEQ ID NO:53)

| | |
|---|---|
| as 908: | CmUmUmCmUmC*C*A*C*C*A*G*C*C*T*UmCmAmGmGm |
| (927-908) | |

T1-1 cells were plated in 96 well plate, 10⁴ cells/well in DMEM, 10% FCS. 24 hours after plating cells were transfected with the following concentrations of each one of the oligonucleotides as described above: 500 nM, 100 nM, 20 nM, using oligofectamin (Invitrogen), according to the recommendations of the manufacturer.

Cells were harvested following 24, 48 and 65 hours post transfection as follows: Medium was aspirated and replaced by 100 microliters PBS and plates were stored at −80° C.

Plates underwent three cycles of freezing and thawing, centrifuged and then cell lysates were stored at −80° C. The quantity of heparanase was determined by ELISA, using 20 microliters of cell lysates as follows: Plates were coated with the heparanase specific monoclonal antibody 3/17. This antibody was raised against a synthetic peptide (pep9-RPGKKVWLGETSSAY, (SEQ ID NO: 54) residues 334-348 on SEQ ID NO 10), which contains the nucleophilic residue of the active site. This antibody binds native heparanase in ELISA and immunoprecipitation assays as well as denatured heparanase in Western blot assays.

The plates were then blocked with 0.5% FCS in PBS-Tween for 1 hour. Samples and heparanase standard diluted in PBS-Tween were applied and incubated overnight at 4° C. Recombinant human heparanase expressed and purified from CHO cells (as described in U.S. Pat. Nos. 6,475,763, 6,426,209 and 6,348,344: Genetically modified cells and methods for expressing recombinant heparanase and methods of purifying same, all of which are incorporated by reference as if fully set forth herein) was used as a standard. Captured heparanase was reacted with biotin labeled affinity purified polyclonal goat anti heparanase antibodies for 30 minutes at 37° C. Labeled antibody was detected by Neutravidin-HRP (Pierce) (15 minutes at 37° C.) using TMB (Pierce) as substrate. Reaction was terminated by H₂S and absorbance at 450/630 nm was measured. Heparanase levels in antisense treated cells at various time points were compared to control cells.

Results

Table 1 shows the relative heparanase quantities in T1-1 clone cells treated with various heparanase specific antisense oligonucleotides at the indicated concentrations (each row shows results for a particular oligonucleotide according to the present invention). Quantities are expressed as percentage of the quantity of heparanase obtained from control, mock transfected cells. As shown, treatment with each of the antisense oligonucleotides resulted in decreased heparanase levels, in a dose dependent manner, although treatment with the antisense oligonucleotides as817 and as908 showed the greatest effect. The inhibitory effect is greatest 24 hours after treatment while a decreased effect is observed after 65 hours.

TABLE 1

| | Concentration of transfected oligonucleotide | | | | | | | | |
|---|---|---|---|---|---|---|---|---|---|
| | 500 | 100 | 20 | 500 | 100 | 20 | 500 | 100 | 20 |
| | nM | nM | nM | nM | nM | nM | nM | nM | nM |
| | Time after treatment | | | | | | | | |
| | 24 hrs | | | 48 hrs | | | 65 hrs | | |
| as908 | 59 | 81 | 94 | 69 | 82 | 77 | 77 | 86 | 76 |
| as817 | 66 | 74 | 95 | 68 | 60 | 74 | 72 | 86 | 90 |
| as681 | 78 | 81 | 95 | 79 | 83 | 85 | 80 | 92 | 103 |
| as511 | 81 | 83 | 88 | 74 | 76 | 74 | 79 | 94 | 104 |
| as312 | 79 | 88 | 102 | 84 | 84 | 88 | 85 | 100 | 106 |
| as175 | 82 | 93 | 89 | 80 | 90 | 89 | 88 | 99 | 105 |

The clearly dose-dependent activity of the antisense oligonucleotides and their ability to alter heparanase activity in cells clearly shows that the antisense oligonucleotides are both capable of entering the cells and of having the desired effect once introduced to the cells.

These results show that the antisense oligonucleotide according to the present invention can be used to treat a subject suffering from a pathological condition, in which the pathological condition is characterized by heparanase activity, which may optionally be over or under expression of heparanase, and which preferably is over expression of heparanase. The method preferably includes administering the antisense oligonucleotide of the present invention to the subject.

Non-limiting examples of the pathological condition may optionally include types of cancers which are characterized by impaired (over) expression of heparanase, and are dependent on the expression of heparanase for proliferating or forming metastases. Therefore, the present invention also encompasses the treatment of cancer, particularly a heparanase-dependent cancer, in which the latter may optionally include any type of cancer for which proliferation and/or metastatic formation is affected by heparanase.

According to another embodiment of the present invention, the antisense oligonucleotide is used to treat other pathological conditions, including but not limited to, autoimmune reactions and inflammation.

It should be noted that the term “treatment” also includes amelioration or alleviation of a pathological condition and/or one or more symptoms thereof, curing such a condition, or preventing the genesis of such a condition.

The present invention also optionally and preferably encompasses modifications to the antisense oligonucleotides, for example for improved delivery through a route of administration, such as oral administration for example, as described in greater detail below.

The present invention also optionally and preferably encompasses pharmaceutical compositions which are suitable for administration of the antisense oligonucleotides of the present invention. Such compositions are known in the art, and are disclosed for example in U.S. Pat. No. 6,153,595, issued on Nov. 28, 2000; and U.S. Pat. No. 5,595,978, issued on Jan. 21, 1997; all of which are hereby incorporated by reference as if fully set forth herein.

As described in the background art, for example in the above patents, the antisense oligonucleotides may optionally be administered according to any suitable route, as is known in the art. Optionally, certain chemical modifications, particularly modifications at the 2′ position of one or more sugar moieties, may be made to the antisense oligonucleotides of the present invention, as such modifications are believed to be particularly useful for stabilizing oligonucleotides for oral administration. Oligonucleotides with 2′-methoxyethoxy modifications are believed to be especially suitable for oral administration. Another suitable route of administration, parenteral administration, includes but is not limited to intravenous drip, infusion or injection, subcutaneous, intraperitoneal and intramuscular injection, pulmonary administration, e.g., by inhalation or insufflation, and intrathecal or intraventricular administration.

Formulations for topical administration may include but are not limited to transdermal patches, implants, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration may include but are not limited to powders or granules, suspensions or solutions in aqueous or non-aqueous media, capsules, sachets or tablets. Thickeners, flavoring agents, diluents, emulsifiers, dispersing aids or binders may also be included.

Pharmaceutically acceptable carriers include, but are not limited to saline solutions and buffered solutions. Suitable pharmaceutically acceptable carriers are well known in the art and are described for example in Gennaro, Alfonso, Ed., Remington's Pharmaceutical Sciences, 18th Edition 1990. Mack Publishing Co., Easton, Pa., a standard reference text in this field. Pharmaceutical carriers may be selected in accordance with the intended route of administration and the standard pharmaceutical practice. For example, for intravenous administration, a saline solution is preferred.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on IC₅₀ S (concentration giving 50% inhibition) or EC₅₀ S (concentration which is 50% effective) found to be effective in in vitro and in vivo animal models. In general, dosage is from about 0.01 micrograms to about 100 g per kg of body weight, and may be given once or more daily, weekly, monthly or yearly, or even once every 2 to 20 years.

The antisense oligonucleotide according to the present invention optionally and preferably includes a polynucleotide or a polynucleotide analog of at least 10 bases being hybridizable in vivo, under physiological conditions, with a portion of a polynucleotide strand encoding a polypeptide having heparanase catalytic activity. Optionally and more preferably, the polynucleotide strand encoding the polypeptide having heparanase catalytic activity is as set forth in SEQ ID NOs: 9, 13, 42, or 43. Also optionally and more preferably, the polypeptide having heparanase catalytic activity is as set forth in SEQ ID NOs: 10, 14 and 44.

Most preferably, the antisense nucleotide has a sequence as set forth in any one of SEQ ID NOs: 48-53;.

According to another embodiment of the present invention, there is provided an antisense oligonucleotide according to the present invention which optionally and preferably includes a polynucleotide or a polynucleotide analog of at least 10 bases being hybridizable in vivo, under physiological conditions, with a portion of a polynucleotide strand being characterized by forming at least a portion of a UTR (untranslated region) for a polynucleotide strand encoding a polypeptide having heparanase catalytic activity.

Most preferably, the UTR has a sequence according to a 5′ UTR for nucleotides 2635-2742 of SEQ ID NO 42 (human) or nucleotides 431-593 of SEQ ID NO 43 (mouse). Alternatively, the UTR may have a sequence according to a 3′ UTR from SEQ ID NO 9, nucleotides 1695-1721; SEQ ID NO 13, nucleotides 1873-1899; SEQ ID NO 42, nucleotides 41864-41890 (human); or SEQ ID NO 43, nucleotides 2199-2396.

The present invention also optionally and preferably features an antisense nucleic acid construct which includes a polynucleotide sequence functioning as a promoter, in which the polynucleotide sequence is derived from SEQ ID NO:42 and includes at least nucleotides 2535-2635 thereof or from SEQ ID NO:43 and includes at least nucleotides 330-430. Also optionally and preferably, the antisense nucleic acid constructs of the present invention may be used for any use of the antisense oligonucleotides of the present invention and/or with any pharmaceutical composition, for example as described above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### LIST OF REFERENCES

1. Wight, T. N., Kinsella, M. G., and Qwamstromn, E. E. (1992). The role of proteoglycans in cell adhesion, migration and proliferation. Curr. Opin. Cell Biol., 4, 793-801.

2. Jackson, R. L., Busch, S. J., and Cardin, A. L. (1991). Glycosaminoglycans: Molecular properties, protein interactions and role in physiological processes. Physiol Rev., 71, 481-539.

3. Wight, T. N. (1989). Cell biology of arterial proteoglycans. Arteriosclerosis, 9, 1-20.

4. Kjellen, L., and Lindahl, U. (1991). Proteoglycans: structures and interactions. Annu. Rev. Biochem., 60, 443-475.

5. Ruoslahti, E., and Yamaguchi, Y. (1991). Proteoglycans as modulators of growth factor activities. Cell, 64, 867-869.

6. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127.

7. Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14, 290-302.

8. Nakajima, M., Irimura, T., and Nicolson, G. L. (1988). Heparanase and tumor metastasis. J. Cell. Biochem., 36, 157-167.

9. Nicolson, G. L. (1988). Organ specificity of tumor metastasis: Role of preferential adhesion, invasion and growth of malignant cells at specific secondary sites. Cancer Met. Rev., 7, 143-188.

10. Liotta, L. A., Rao, C. N., and Barsky, S. H. (1983). Tumor invasion and the extracellular matrix. Lab. Invest., 49, 639-649.

11. Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711.

[symbol] 12 [symbol] . Vlodavsky, I., Ishai-Michaeli, R., Bar-Ner, M., Fridman, R., Horowitz, A. T., Fuks, Z. and Biran, S. (1988). Involvement of heparanase in tumor metastasis and angiogenesis. Is. J. Med., 24, 464-470.

13. Vlodavsky, I., Liu, G. M., and Gospodarowicz, D. (1980). Morphological appearance, growth behavior and migratory activity of human tumor cells maintained on extracellular matrix vs. plastic. Cell, 19, 607-616.

14. Gospodarowicz, D., Delgado, D., and Vlodavsky, I. (1980). Permissive effect of the extracellular matrix on cell proliferation in-vitro. Proc. Natl. Acad. Sci. USA., 77, 4094-4098.

15. Bashkin, P., Doctrow, S., Klagsbrun, M., Svahn, C. M., Folkman, J., and Vlodavsky, I. (1989). Basic fibroblast growth factor binds to subendothelial extracellular matrix and is released by heparitinase and heparin-like molecules. Biochemistry, 28, 1737-1743.

16. Parish, C. R., Coombe, D. R., Jakobsen, K. B., and Underwood, P. A. (1987). Evidence that sulphated polysaccharides inhibit tumor metastasis by blocking tumor cell-derived heparanase. Int. J. Cancer, 40, 511-517.

16a. Vlodavsky, I., Hua-Quan Miao., Benezra, M., Lider, O., Bar-Shavit, R., Schmidt, A., and Peretz, T. (1997). Involvement of the extracellular matrix, heparan sulfate proteoglycans and heparan sulfate degrading enzymes in angiogenesis and metastasis. In: Tumor Angiogenesis. Eds. C. E. Lewis, R. Bicknell & N. Ferrara. Oxford University Press, Oxford UK, pp. 125-140.

17. Burgess, W. H., and Maciag, T. (1989). The heparin-binding (fibroblast) growth factor family of proteins. Annu. Rev. Biochem., 58, 575-606.

18. Folkman, J., and Klagsbrun, M. (1987). Angiogenic factors. Science, 235, 442-447.

19. Vlodavsky, I., Folkman, J., Sullivan, R., Fridman, R., Ishai-Michaelli, R., Sasse, J., and Klagsbrun, M. (1987). Endothelial cell-derived basic fibroblast growth factor: Synthesis and deposition into subendothelial extracellular matrix. Proc. Natl. Acad. Sci. USA, 84, 2292-2296.

20. Folkman, J., Klagsbrun, M., Sasse, J., Wadzinski, M., Ingber, D., and Vlodavsky, I. (1980). A heparin-binding angiogenic protein—basic fibroblast growth factor—is stored within basement membrane. Am. J. Pathol., 130, 393-400.

21. Cardon-Cardo, C., Vlodavsky, I., Haimovitz-Friedman, A., Hicklin, D., and Fuks, Z. (1990). Expression of basic fibroblast growth factor in normal human tissues. Lab. Invest., 63, 832-840.

22. Ishai-Michaeli, R., Svahn, C.-M., Chajek-Shaul, T., Komer, G., Ekre, H.-P., and Vlodavsky, I. (1992). Importance of size and sulfation of heparin in release of basic fibroblast factor from the vascular endothelium and extracellular matrix. Biochemistry, 31, 2080-2088.

23. Ishai-Michaeli, R., Eldor, A., and Vlodavsky, I. (1990). Heparanase activity expressed by platelets, neutrophils and lymphoma cells releases active fibroblast growth factor from extracellular matrix. Cell Reg., 1, 833-842.

24. Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271.

25. Vlodavsky, I., Bar-Shavit, R., Komer, G., and Fuks, Z. (1993). Extracellular matrix-bound growth factors, enzymes and plasma proteins. In Basement membranes: Cellular and molecular aspects (eds. D. H. Rohrbach and R. Timpl), pp327-343. Academic press Inc., Orlando, Fla.

26. Yayon, A., Klagsbrun, M., Esko, J. D., Leder, P., and Ornitz, D. M. (1991). Cell surface, heparin-like molecules are required for binding of basic fibroblast growth factor to its high affinity receptor. Cell, 64, 841-848.

27. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible for FGF receptor dimerization, activation, and cell proliferation. Cell, 79, 1015-1024.

28. Ornitz, D. M., Herr, A. B., Nilsson, M., West, a., J., Svahn, C.-M., and Waksman, G. (1995). FGF binding and FGF receptor activation by synthetic heparan-derived di- and trisaccharides. Science, 268, 432-436.

29. Gitay-Goren, H.,. Soker, S., Vlodavsky, I., and Neufeld, G. (1992). Cell surface associated heparin-like molecules are required for the binding of vascular endothelial growth factor (VEGF) to its cell surface receptors. J. Biol. Chem., 267, 6093-6098.

30. Lider, O., Baharav, E., Mekori, Y., Miller, T., Naparstek, Y., Vlodavsky, I., and Cohen, I. R. (1989). Suppression of experimental autoimmune diseases and prolongation of allograft survival by treatment of animals with heparinoid inhibitors of T lymphocyte heparanase. J. Clin. Invest., 83, 752-756.

31. Lider, O., Cahalon, L., Gilat, D., Hershkovitz, R., Siegel, D., Margalit, R., Shoseyov, O., and Cohn, I. R. (1995). A disaccharide that inhibits tumor necrosis factor a is formed from the extracellular matrix by the enzyme heparanase. Proc. Natl. Acad. Sci. USA., 92, 5037-5041.

31a. Rapraeger, A., Krufka, A., and Olwin, B. R. (1991). Requirement of heparan sulfate for bFGF-mediated fibroblast growth and myoblast differentiation. Science, 252, 1705-1708.

32. Eisenberg, S., Sehayek, E., Olivecrona, T., and Vlodavsky, I. (1992). Lipoprotein lipase enhances binding of lipoproteins to heparan sulfate on cell surfaces and extracellular matrix. J. Clin. Invest., 90, 2013-2021.

[symbol] 33 [symbol] . Shieh, M-T., Wundunn, D., Montgomery, R. I., Esko, J. D., and Spear, P. G. J. (1992). Cell surface receptors for herpes simplex virus are heparan sulfate proteoglycans. J Cell Biol., 116, 1273-1281.

33a. Chen, Y., Maguire, T., Hileman, R. E., Fromm, J. R., Esko, J. D., Linhardt, R. J., and Marks, R. M. (1997). Dengue virus infectivity depends on envelope protein binding to target cell heparan sulfate. Nature Medicine 3, 866-871.

33b. Putnak, J. R., Kanesa-Thasan, N., and Innis, B. L. (1997). A putative cellular receptor for dengue viruses. Nature Medicine 3, 828-829.

[symbol] 34 [symbol] . Narindrasorasak, S., Lowery, D., Gonzalez-DeWhitt, P., Poorman, R. A., Greenberg, B., Kisilevsky, R. (1991). High affinity interactions between the Alzheimer's beta-amyloid precursor protein and the basement membrane form of theparan sulfate proteoglycan. J. Biol. Chem., 266, 12878-83.

35. Ross, R. (1993). The pathogenesis of atherosclerosis: a perspective for the 1990s. Nature (Lond.)., 362:801-809.

36. Zhong-Sheng, J., Walter, J., Brecht, R., Miranda, D., Mahmood Hussain, M., Innerarity, T. L. and Mahley, W. R. (1993). Role of heparan sulfate proteoglycans in the binding and uptake of apolipoprotein E-enriched remnant lipoproteins by cultured cells. J. Biol. Chem., 268, 10160-10167.

37. Ernst, S., Langer, R., Cooney, Ch. L., and Sasisekharan, R. (1995). Enzymatic degradation of glycosaminoglycans. Critical Reviews in Biochemistry and Molecular Biology, 30(5), 387-444.

38. Gospodarowicz, D., Mescher, A L., Birdwell, C R. (1977). Stimulation of corneal endothelial cell proliferation in vitro by fibroblast and epidermal growth factors. Exp Eye Res 25, 75-89.

39. Haimovitz-Friedman, A., Falcone, D. J., Eldor, A., Schirrmacher, V., Vlodavsky, I., and Fuks, Z. (1991) Activation of platelet heparitinase by tumor cell-derived factors. Blood, 78, 789-796.

39a. Savitsky, K., Platzer, M., Uziel, T., Gilad, S., Sartiel, A., Rosental, A., Elroy-Stein, O., Siloh, Y. and Rotman, G. (1997). Ataxia-telangiectasia: structural diversity of untranslated sequences suggests complex post-translational regulation of ATM gene expression. Nucleic Acids Res. 25(9), 1678-1684.

40. Bar-Ner, M., Eldor, A., Wasserman, L., Matzner, Y., and Vlodavsky, I. (1987). Inhibition of heparanase mediated degradation of extracellular matrix heparan sulfate by modified and non-anticoagulant heparin species. Blood, 70, 551-557.

41. Goshen, R., Hochberg, A., Komer, G., Levi, E., Ishai-Michaeli, R., Elkin, M., de Grot, N., and Vlodavsky, I. (1996). Purification and characterization of placental heparanase and its expression by cultured cytotrophoblasts. Mol. Human Reprod., 2, 679-684.

42. Korb M., Ke Y. and Johnson L. F. (1993) Stimulation of gene expression by introns: conversion of an inhibitory intron to a stimulatory intron by alteration of the splice donor sequence. Nucleic Acids Res., 25;21(25):5901-8.

43. Zheng B., Qiu X. Y., Tan M., Xing Y. N., Lo D., Xue J. L. and Qiu X. F. (1997) Increment of hFIX expression with endogenous intron 1 in vitro. Cell Res., 7(1):21-29.

44. Kurachi S., Hitomi Y., Furukawa M. and Kurachi K. (1995) Role of intron I in expression of the human factor IX gene. J. Biol. Chem. 10, 270(10):5276-5281.

45. Shekhar P. V. and Miller F. R. (1994-5) Correlation of differences in modulation of ras expression with metastatic competence of mouse mammary tumor subpopulations. Invasion Metastasis, 14(1-6):27-37.

46. Zhou G., Garofalo S., Mukhopadhyay K., Lefebvre V., Smith C. N., Eberspaecher H. and de Crombrugghe B. (1995) A 182 bp fragment of the mouse pro alpha 1(II) collagen gene is sufficient to direct chondrocyte expression in transgenic mice. J. Cell Sci., 108 (Pt 12):3677-3684.

47. Hormuzdi S. G., Penttinen R., Jaenisch R. and Bornstein P. (1998) A gene-targeting approach identifies a function for the first intron in expression of the alpha1(I) collagen gene. Mol. Cell, 18(6):3368-3375.

48. Kang Y. K., Lee C. S., Chung A. S. and Lee K. K. (1998) Prolactin-inducible enhancer activity of the first intron of the bovine beta-casein gene. Mol. Cells,. 30;8(3):259-265.

49. Chow Y. H., O'Brodovich H., Plumb J., Wen Y., Sohn K. J., Lu Z., Zhang F., Lukacs G. L., Tanswell A. K., Hui C. C., Buchwald M. and Hu J. (1997) Development of an epithelium-specific expression cassette with human DNA regulatory elements for transgene expression in lung airways. Proc. Natl. Acad. Sci. USA, 23;94(26):14695-14700.

50. Gottschalk U. and Chan S. (1998) Somatic gene therapy. Present situation and future perspective. Arzneimittelforschung, 48(11): 1111-1120.

51. Ye S., Cole-Strauss A. C., Frank B. and Kmiec E. B. (1998) Targeted gene correction: a new strategy for molecular medicine. Mol. Med. Today, 4(10):431-437.

52. Lai L., and Lien Y. (1999) Homologous recombination based gene therapy. Exp. Nephrol., 7(1):11-14.

53. Yazaki N., Fujita H., Ohta M., Kawasaki T. and Itoh N. (1993) The structure and expression of the FGF receptor-1 mRNA isoforms in rat tissues. Biochim. Biophys. Acta., 20;1172(1-2):37-42.

54. Le Fur N., Kelsall S. R., Silvers W. K. and Mintz B. (1997) Selective increase in specific alternative splice variants of tyrosinase in murine melanomas: a projected basis for immunotherapy. Proc. Natl. Acad. Sci. USA, 13;94(10):5332-5337.

55. Miyake H., Okamoto I., Hara I., Gohji K., Yamanaka K., Arakawa S., Kamidono S. and Saya H. (1998) Highly specific and sensitive detection of malignancy in urine samples from patients with urothelial cancer by CD44v8-10/CD44v10 competitive RT-PCR. Int. J. Cancer, 18;79(6):560-564.

56. Guriec N., Marcellin L., Gairard B., Calderoli H., Wilk A., Renaud R., Bergerat J. P. and Oberling F. (1996) CD44 exon 6 expression as a possible early prognostic factor in primary node negative breast carcinoma. Clin. Exp. Metastasis, 14(5):434-439.

57. Gewirtz A. M., Sokol D. L. and Ratajczak M. Z. (1998) Nucleic acid therapeutics: state of the art and future prospects. Blood, 1;92(3):712-736.

58. Hida K., Shindoh M., Yasuda M., Hanzawa M., Funaoka K., Kohgo T., Amemiya A., Totsuka Y., Yoshida K. and Fujinaga K (1997) Antisense E1AF transfection restrains oral cancer invasion by reducing matrix metalloproteinase activities. Am. J. Pathol. 150(6) :2125-2132.

59. Shastry B. S. (1998) Gene disruption in mice: models of development and disease. Mol. Cell. Biochem. 1998 April;181(1-2):163-179.

60. Carpentier A. F., Rosenfeld M. R., Delattre J. Y., Whalen R. G., Posner J. B. and Dalmau J. (1998) DNA vaccination with HuD inhibits growth of a neuroblastoma in mice. Clin. Cancer Res., 4(11):2819-2824.

61. Lai W. C. and Bennett M. (1998) DNA vaccines. Crit. Rev. Immunol., 18(5):449-484.

62. Welch P. J., Barber J. R., and Wong-Staal F. (1998) Expression of ribozymes in gene transfer systems to modulate target RNA levels. Curr. Opin. Biotechnol., 9(5):486-496.

63. Durand P., Lehn P., Callebaunt I., Fabrega S., Henrissat B. and Momon J. P. (1997) Active-site motifs of lysosomal acid hydrolyses: invariant features of clan GH-A glycosyl hydrolases deduced from hydrophobic cluster analysis. Glycobiology, 7(2):277-284.

64. Thuong and Helene (1993) Sequence specific recognition and modification of double helical DNA by oligonucleotides Angev. Chem. Int. Ed. Engl. 32:666

65. Dash P., Lotan I., Knapp M., Kandel E. R. and Goelet P. (1987) Selective elimination of mRNAs in vivo: complementary oligodeoxynucleotides promote RNA degradation by an RNase H-like activity. Proc. Natl. Acad. Sci. USA, 84:7896.

66. Chiang M. Y., Chan H., Zounes M. A., Freier S. M., Lima W. F. and Bennett C. F. (1991) Antisense oligonucleotides inhibit intercellular adhesion molecule 1 expression by two distinct mechanisms. J. Biol. Chem. 266:18162-71.

67. Paterson Paterson B. M, Roberts B. E and Kuff EL . (1977) Structural gene identification and mapping by DNA-mRNA hybrid-arrested cell-free translation. Proc. Natl. Acad. Sci. USA, 74:4370.

68. Cohen (1992) Oligonucleotide therapeutics. Trends in Biotechnology, 10:87.

69. Szczylik et al (1991) Selective inhibition of leukemia cell proliferation by BCR-ABL antisense oligodeoxynucleotides. Science 253:562.

70. Calabretta et al. (1991) Normal and leukemic hematopoietic cell manifest differential sensitivity to inhibitory effects of c-myc antisense oligodeoxynucleotides: an in vitro study relevant to bone marrow purging. Proc. Natl. Acad. Sci. USA 88:2351.

71. Heikhila et al. (1987) A c-myc antisense oligodeoxynucleotide inhibits entry into S phase but not progress from G(0) to G(1). Nature, 328:445.

72. Reed et al. (1990) Antisense mediated inhibition of BCL2 prooncogene expression and leukemic cell growth and survival: comparison of phosphodiester and phosphorothioate oligodeoxynucleotides. Cancer Res. 50:6565.

73. Burch and Mahan (1991) Oligodeoxynucleotides antisense to the interleukin I receptor m RNA block the effects of interleukin I in cultured murine and human fibroblasts and in mice. J. Clin. Invest. 88:1190.

74. Agrawal (1992) Antisense oligonucleotides as antiviral agents. TIBTECH 10:152.

75. Uhlmann et al. (1990) Chem. Rev. 90:544.

76. Cook (1991) Medicinal chemistry of antisense oligonucleotides—future opportunities. Anti-Cancer Drug Design 6:585.

77. Biotechnology research news (1993) Can DNA mimics improve on the real thing? Science 262:1647.

What is claimed is:

1. An antisense oligonucleotide comprising a polynucleotide or a polynucleotide analog of at least 10 bases being hybridizable in vivo, under physiological conditions, with a portion of a polynucleotide strand encoding a polypeptide having heparanase catalytic activity.

2. The antisense oligonucleotide of claim 1, wherein said polynucleotide strand encoding said polypeptide having heparanase catalytic activity is as set forth in SEQ ID NOs: 9, 13, 42, or 43.

3. The antisense oligonucleotide of claim 1, wherein said polypeptide having heparanase catalytic activity is as set forth in SEQ ID NOs: 10, 14 and 44.

4. The antisense oligonucleotide of claim 1, having a sequence as set forth in SEQ ID NOs: 48; 49; 50; 51; 52; or 53

5. A method of in vivo downregulating heparanase activity comprising administering the antisense oligonucleotide of claim 1 in vivo.

6. A method of treating a subject suffering from a pathological condition, said pathological condition being characterized by heparanase activity, the method comprising administering the antisense oligonucleotide of claim 1 to the subject.

7. The method of claim 6, wherein said pathological condition comprises a heparanase-dependent cancer.

8. The method of claim 6, wherein said pathological condition comprises cancer.

9. The method of claim 6, wherein said pathological condition comprises an autoimmune reaction.

10. The method of claim 6, wherein said pathological condition comprises inflammation.

11. A pharmaceutical composition comprising the antisense oligonucleotide of claim 1 and a pharmaceutically acceptable carrier.

12. An antisense nucleic acid construct comprising a promoter sequence and a polynucleotide sequence directing the synthesis of an antisense RNA sequence of at least 10 bases being hybridizable in vivo, under physiological conditions, with a polynucleotide strand encoding a polypeptide having heparanase catalytic activity.

13. The antisense nucleic acid construct of claim 12, wherein said polynucleotide strand encoding said polypeptide having heparanase catalytic activity is as set forth in SEQ ID NOs: 9, 13, 42 or 43.

14. The antisense nucleic acid construct of claim 12, wherein said polypeptide having heparanase catalytic activity is as set forth in SEQ ID NOs: 10, 14 or 44.

15. The antisense nucleic acid construct of claim 12, comprising a polynucleotide sequence functioning as a promoter, said polynucleotide sequence is derived from SEQ ID NO:42 and includes at least nucleotides 2535-2635 thereof or from SEQ ID NO:43 and includes at least nucleotides 330-430.

16. A method of in vivo downregulating heparanase activity comprising the step of in vivo administering the antisense nucleic acid construct of claim 12.

17. A pharmaceutical composition comprising the antisense nucleic acid construct of claim 12 and a pharmaceutically acceptable carrier.

18. An antisense oligonucleotide, comprising a polynucleotide or a polynucleotide analog of at least 10 bases being hybridizable in vivo, under physiological conditions, with a portion of a polynucleotide strand being characterized by forming at least a portion of a UTR (untranslated region) for a polynucleotide strand encoding a polypeptide having heparanase catalytic activity.

19. The antisense oligonucleotide of claim 18, wherein said UTR comprises a sequence according to a 5′ UTR for nucleotides 2635-2742 of SEQ ID NO 42 (human) or nucleotides 431-593 of SEQ ID NO 43 (mouse).

20. The antisense oligonucleotide of claim 18, wherein said UTR comprises a sequence according to a 3′ UTR from SEQ ID NO 9, nucleotides 1695-1721; SEQ ID NO 13, nucleotides 1873-1899; SEQ ID NO 42, nucleotides 41864-41890 (human); or SEQ ID NO 43, nucleotides 2199-2396.


SEQUENCE LISTING (54 sequences)

SEQ ID NO: 1 - DNA, 27 bp, Artificial sequence
  Single strand DNA oligonucleotide
ccatcctaatacgactcactatagggc

SEQ ID NO: 2 - DNA, 24 bp, Artificial sequence
  Single strand DNA oligonucleotide
gtagtgatgccatgtaactgaatc

SEQ ID NO: 3 - DNA, 23 bp, Artificial sequence
  Single strand DNA oligonucleotide
actcactatagggctcgagcggc

SEQ ID NO: 4 - DNA, 22 bp, Artificial sequence
  Single strand DNA oligonucleotide
gcatcttagccgtctttcttcg

SEQ ID NO: 5 - DNA, 15 bp, Artificial sequence
  Single strand DNA oligonucleotide
ttttttttttttttt

SEQ ID NO: 6 - DNA, 23 bp, Artificial sequence
  Single strand DNA oligonucleotide
ttcgatcccaagaaggaatcaac

SEQ ID NO: 7 - DNA, 24 bp, Artificial sequence
  Single strand DNA oligonucleotide
gtagtgatgccatgtaactgaatc

SEQ ID NO: 8 - protein, 9 aa, Homo sapiens
YGPDVGQPR

SEQ ID NO: 9 - DNA, 1721 bp, Homo sapiens
ctagagctttcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 10 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 11 - DNA, 1721 bp, Homo sapiens; identical to SEQ ID NO: 9
  CDS (63)..(1691)

SEQ ID NO: 12 - DNA, 824 bp, Mus musculus
ctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 13 - DNA, 1899 bp, Homo sapiens
gggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 14 - protein, 592 aa, Homo sapiens
MEGAVGGVRRRNGAEERRKGRWGSAGGSARALDSPLRGSWRGEQPGEPKMLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 15 - DNA, 1899 bp, Homo sapiens; identical to SEQ ID NO: 13
  CDS (94)..(1869)

SEQ ID NO: 16 - DNA, 594 bp, Homo sapiens
attactatagggcacgcgtggtcgacggcccgggctggtattgtcttaatgagaagttgataaagaattttgggtggttgatctctttccagctgcagtttagcgtatgctgaggccagattttttcaggcaaaagtaaaatacctgagaaactgcctggccagaggacaatcagattttggctggctcaagtgacaagcaagtgtttataagctagatgggagaggaagggatgaatactccattggaggctttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccggggggaaagcgagcaaggaagtaggagagagccgggcaggcggggcggggttggattgggagcagtgggagggatgcagaagaggagtgggaggg

SEQ ID NO: 17 - DNA, 21 bp, Artificial sequence
  Single strand DNA oligonucleotide
ccccaggagcagcagcatcag

SEQ ID NO: 18 - DNA, 21 bp, Artificial sequence
  Single strand DNA oligonucleotide
aggcttcgagcgcagcagcat

SEQ ID NO: 19 - DNA, 22 bp, Artificial sequence
  Single strand DNA oligonucleotide
gtaatacgactcactatagggc

SEQ ID NO: 20 - DNA, 19 bp, Artificial sequence
  Single strand DNA oligonucleotide
actatagggcacgcgtggt

SEQ ID NO: 21 - DNA, 21 bp, Artificial sequence
  Single strand DNA oligonucleotide
cttgggctcacctggctgctc

SEQ ID NO: 22 - DNA, 23 bp, Artificial sequence
  Single strand DNA oligonucleotide
agctctgtagatgtgctatacac

SEQ ID NO: 23 - DNA, 22 bp, Artificial sequence
  Single strand DNA oligonucleotide
gcatcttagccgtctttcttcg

SEQ ID NO: 24 - DNA, 23 bp, Artificial sequence
  Single strand DNA oligonucleotide
gagcagccaggtgagcccaagat

SEQ ID NO: 25 - DNA, 23 bp, Artificial sequence
  Single strand DNA oligonucleotide
ttcgatcccaagaaggaatcaac

SEQ ID NO: 26 - DNA, 23 bp, Artificial sequence
  Single strand DNA oligonucleotide
agctctgtagatgtgctatacac

SEQ ID NO: 27 - DNA, 24 bp, Artificial sequence
  Single strand DNA oligonucleotide
tcagatgcaagcagcaactttggc

SEQ ID NO: 28 - DNA, 22 bp, Artificial sequence
  Single strand DNA oligonucleotide
gcatcttagccgtctttcttcg

SEQ ID NO: 29 - DNA, 24 bp, Artificial sequence
  Single strand DNA oligonucleotide
gtagtgatgccatgtaactgaatc

SEQ ID NO: 30 - DNA, 22 bp, Artificial sequence
  Single strand DNA oligonucleotide
aggcaccctagagatgttccag

SEQ ID NO: 31 - DNA, 24 bp, Artificial sequence
  Single strand DNA oligonucleotide
gaagatttctgtttccatgacgtg

SEQ ID NO: 32 - DNA, 25 bp, Artificial sequence
  Single strand DNA oligonucleotide
ccacactgaatgtaatactgaagtg

SEQ ID NO: 33 - DNA, 22 bp, Artificial sequence
  Single strand DNA oligonucleotide
cgaagctctggaactcggcaag

SEQ ID NO: 34 - DNA, 22 bp, Artificial sequence
  Single strand DNA oligonucleotide
gccagctgcaaaggtgttggac

SEQ ID NO: 35 - DNA, 23 bp, Artificial sequence
  Single strand DNA oligonucleotide
aacacctgcctcatcacgacttc

SEQ ID NO: 36 - DNA, 22 bp, Artificial sequence
  Single strand DNA oligonucleotide
gccaggctggcgtcgatggtga

SEQ ID NO: 37 - DNA, 22 bp, Artificial sequence
  Single strand DNA oligonucleotide
gtcgatggtgatggacaggaac

SEQ ID NO: 38 - DNA, 22 bp, Artificial sequence
  Single strand DNA oligonucleotide
gtaatacgactcactatagggc

SEQ ID NO: 39 - DNA, 19 bp, Artificial sequence
  Single strand DNA oligonucleotide
actatagggcacgcgtggt

SEQ ID NO: 40 - DNA, 27 bp, Artificial sequence
  Single strand DNA oligonucleotide
ccatcctaatacgactcactatagggc

SEQ ID NO: 41 - DNA, 23 bp, Artificial sequence
  Single strand DNA oligonucleotide
actcactatagggctcgagcggc

SEQ ID NO: 42 - DNA, 44848 bp, Homo sapiens
ggatcttggctcactgcaatctctgcctcccatgcaattcttatgcatcagcctcctgagtagcttggattataggtctgcgccaccactcctggctacaccatgttgcccaggctggtcttgaactcttgggctctagtgatccacccgccttggcctcccaaagtgctgggattacaggtgtgagccatcacacccggccccccgtttccatattagtaactcacatgtagaccacaaggatgcactatttagaaaacttgcaatggtccacttttcaaatcacccaaacatgttaaagaaattggtatgactgggcatggcacagtggctcatgcctgcaatcctagcattttgtgaggctgagacgggcagatcacgaggtcaggagattgagaccatcctgacagacatggtgaaatcccatctctactaaaaatacaaaacaattagccgggggtgatggcaggcccctgtagtcccagctactcgggaggctgaggcaggagaatggcgtgaatccaggaggcagagcttgcagtgagccgagatggtgccactgcactccagcctgggcgacagagcgagactccgtctcaaaaaaaaaaaaaaagaaagaaattggtatgactgttgactcacaacaggagtcaggggcatggggtggggtgtaagattaatgtcatgacaaatgtggaaaagaaacttctgtttttccaactccacgtctgctaccatattattacactcttctggtagtgtggtgtttatgtgtgaattttttttcatatgtatacagtaattgtaggatatgaacctgattctagttgcaaaactcactatgagcttagcttttaagttgcttaagaataggtagatctatgcaaataatgataattattattattattttaagagagggtctcactttgtcacccaggctggagtgcagtggtgtgattaagggtcactgcaacctccacctcccaggctcaaataaacctcccacctcagcctccccagtagctggaaccacaggcacgggccaccacgcctggctaattttttgtattttttgtagagatggggtttcatcatgttgcccaggctgttcttgaattcctcggctcaagcaatcctcccaccttggcctcccaaaatgctggcatcacaggcatgatggcatcactggcatcacataccatgcctggcctgatttatgcaaattagatatgcatttcaaaataatctatttttatttgttgccttattggtggtacaatctcaagtggaaaaatctaagggttttggtgttatttgcttactcaaccaatatttattagactcttactaagcaccaacatgatcacatgcctgagctatggctagcatagcgtgtgagacaaacttaatctctgttttggtggagcatataatctagtagatgaagccaatgttgagcaacatcacaatactaacaaattgaggatgctacgagagtgtctaacaaattgaggatgctacgagagtgtctaacaaattgaggatgctatgagagtgtgtcatggagagctgcctggagattgagagaaagcttccttgagggaagttacatttcagctgaaacacactgccatctgctcgaggttttgtaactgcattcacatcccgattctgacacttcacatcccgattctgacacttcacccagttactgtctcagagcttgggtccgcatgtgtaaaacaaggacagtatgcacttggcagggttgtgagaagggaagagaacacaagtaaagcacctgtatcaggcatacagtaggcactaagcgtgcgatgcttgctatgattatacatcagtgtaagcatcaaggaaaagctgaagaaaagtctgaccaacagcgaaagataaatgcgcagaggagaaatttggcaaaggctccaaattcaggggcagtccgtactctacactttgtatgggggcttcaggtcctgagttccagacattggagcaactaaccctttaagattgctaaatattgtcttaatgagaagttgataaagaattttgggtggttgatctctttccagctgcagtttagcgtatgctgaggccagattttttcaagcaaaagtaaaatacctgagaaactgcctggccagaggacaatcagattttggctggctcaagtgacaagcaagtgtttataagctagatgggagaggaagggatgaatactccattggaggttttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctggggtaagcgccagcctcctggtcctgtcccctttcctgtcctcctgacacctatgtctgccccgccagcggctctccttcttttgcgcggaaacaacttcacaccggaacctccccgcctgtctctccccaccccacttcccgcctctcattctccctctccctcccttactctcagaccccaaaccgctttttggggggtatcatttaaaaaatagatttaggggttacaagtgcagttctgttccatgggtatattgcattgtggtggcatctgggctcttagtgtaactgtcacccgaatgttgtacattgtatctaataggtaatttctcatccctcatccctctcccaccctcccaccttttggagtctccagtgtctactattccactaagtccatgtgtacacattgtttagcgcccactctaaatgagcctttttgtttcattcattctgtaagtgttgaataggcaccacctaaggtcaggtataagtggaaatttgaaaaagaaactgcccacttgccccagtacttccctagccaagaggagggaaaccaggcaggtgcacctgaaggcctgtgagtgcttgatttgctgtgcagtgtaggacaagtaagattgtgcatagccttctgtatttaagactgtgttaggaagatttctctttcttttcttttctttttcttttttcttttcttttttttttttaggcagatgaaaagggcgtcacagaacaggaataaaaatctaaatattcaataaatgagacctaggagactactgcagtgacttacaaagtcctaataaaaagatgtctctccaaaatggggctgcaaaatgtggtgctgccttatcagctctaagttttttccttacctgagaaagaaggaacctgatgcaggttcagggctcctgccccatgaatgcaggctgactccaagatggggagctacagggacaatcccaggtcttctaggcctcttatttaggccctgggagcctccagagatggccacatcttgaccagcccagatagagggaaagatcaccattatctcacctctgtgtcaaatacctagatgctgtcctccctgagcccacactatagttgccagcgctaatttaatgggtagtgtactggttaagagatggacagaccatcctggcttgactctcagctctggcaaagatgagtgacttggtttttccatatctcttggccacaccaaccttgatttcttcagctgtagaatggaatttctcaagcttgcctcaaggattattgcccgaggatttgatgatatggtaagagcttctcagtgtttgacccatagtaagtgtttgacgtttcaaacgaattgtttctttctaggacatggtgagcatttggtagccattcaccggttttctgtttctttggatcatagttaacctctccttttccttctggcactacaattttctggtggggaagaatccttactttctgcccttccccttaaggataggaagctgatactaggcagcaactagttgggggataggaagattgttccagagaaatgctgaaccatagggctccagatcacaggaccccagtcttagcttgctggggtgtggggtgggggggggcggttactgaacatgggtatgaagtagatgtccatttactgaaatgtgaggacctgaggcctcttctattgctgtagccagcatattccccaacctctccccaagaaaggacagatgggggttcccccctggagtaacaggtccaaaagaaaaaacatacagtgggacttccaggatctgggcctgatcacccagcagtcaagctccccgcaattgactaacacccccctaacacgtagaaattccaatctgcaatttagtgaggatgatacctttattcttcttaaatacatctcttcatttcccagagcacccttttttcccctcctctgcacctttttgttaaagactggagtataatgaaataccaagagagcataacatgtgatacataaaactttttttctggtttacaaaacagttcattcttgtccatacgtgcttctctccaaggctggctgctgtctgttccagcccgcttcgcttggagaggccatctgccatacctgctccccagacgcatcgacaagcacacccagagtgttatctgctaagacctaaaagagggaggaaccccctctcctcatctaagacctagcttctaaattagagtgtgagggtccatctccccaggaggggcacagggcccaaacagcccagccatctcagaagacaacactaagctttgtaggggtccacagtagaggagagtaagacgcctgttgtttaatttattacagttcctcaaaagtgaagatgtgtgggcgggatggcaagagctgagcagacgaaagctgaaggaataaggaaagagaggaggacacaaacagctgacacttcctcagttcttgtcatttgcctggccctgttctaagcaccttctaggtattaatccatttagtcttggctacaacactgtgagtaactagttttgtcacccccattttaaaaatgaagaaagtgaggctcagggaggttaagtaacttggccacagtttgaaactagactctgatcacatgagataatagtgcccataaaaagggaaagcagattatattttttaaaggaaagagagtaggatatggtagaaaaagattgtttggaaaggaattgagagattgatataatgaaaagaagcattcacatgagagtaacagtatcagggcccaaaccttcatctaaggtacttcaaagaggcctaagcaaacttagtcactggcgtggttctagtctccatgatggcaaatacattgtgtacagcccaactccacacaaaacttaaataccaatgatagagcaatctaaaatttgaaagaaaaaatctttcaatttgtcgtcttcccagagggacttaatcaagaaaccaatcaaaatacttcctaagcctaactgtgtgcagaactccaaagagagcccagccctaaatcaacactgtccaatggaaatataatataatgtgggcctcatatgcaaggtcatatgtaattttaaattttctagtagccatattaaaaaggtaaaaagaaacaagtgaaattaattttaataattttatttagttcaatagatccaaaatgttttctcagcatgtaatcaatataaaaatattaatgaggtatttattattccttttctcaaaccaagtctattctataatctggcgtgtattatttacagcacttctcagactatatttctttctttcttttttttttccgagacaattttgctcttgtcacccaagctagagtacaatggcgttacctcggctcactgcaacctccgcctcccgggttcaagttattctcctgcctcagtctcccaagtagctgggactagaggcatgcaccaccacgcctggctaattgtgtatttttagtagagacagggtttcaccatgttggccaggctaatctcaaactcctgagctcaggtgatatgcccacctcggcctcccaaagtgttgggattacaggcgtgagccactgcacccggcctcagattaactatatttcaagcgttcagtagccacatgtagctagtgctatggtagtggacagtacagatctgcatttcaattaagacacgtatacaagcatagttcactaatgcacggtaaaaaaaagtatagtgctgagtcggtggtagaaatcctaaatactgcagagcaaaagtggtacgaacagcaatctcagtgataatgcaaccatgcttgcttttcattgcaatttgcttattttccttcagcaaagttcatccatttttgccaattcaataaatatttactgataaaaactttcaatattagattcttgcatcttcatagacagagttgcttttcacatttagaaaattacttatcaatgttaaacacacgttttgataaccagtgttggaaagaggtgcagactccccatgtgcctattgatggcagaaatattcacagccaaagggaaacaaagggctggggacaatcacacacctcatgtctcctaactcctgggaagtgctgtccctctgattgagctcttattattgccttccccactaaccctgtccactgtgccctggagccctttgcagggttacctgctctgtcctcctcacagaatatctcctctacctccttgtccaagctacaacttggctattctctgatgacactgtcttccctgtagcccttttgagtaatggctgcatattctcccatagtccagttcttttcctgttctccagtctggcttctggatgacagcccactagtttgaactccatactgctatagttcaagtcccttttgacttgttaccttgggcaaattacctccttttgttcaggttccttgtttgtaaaatgacgataataatgccatttgcttcagtgggttattttgaaattgagtgaaagaaggcgggtagcttccctacacgctcagtgtagactagcctgatgtgcattacgggtgatgccatgactcagtgtgttttcctcatctccacatctggctctcatccagtgctcctgcttacggcactctgtccccctcttacttactcccccttattaactgaagactggcactgatctcacagtttcctctccacttcctagtctcaccatcatcctagatgacttcaagtcacctagataaactgtctcagtttcttcactcacatttttttataacagataatgttacactcaagttgtaacagaaccagcttatccagctcatgaaatgtatgcatttcatctcaactctgtattcagtgacatcctgtgggtatctggaaatcagccatggtgagaatatttaccatggaaattggcaaatactaaaaagcagagcacctttttttctgagagccagaccatagctcttctactccatagcacccatcataacaatttttaaatacctccactgaacagcttcttcctctctctacttcttccatatctgatttgagcttcttaatttatcatgtgaaccactcttgtaataataaccccaaatccctgttccattgttcttcctgctaaaatactaaacctggtttagtccaaccatattttctctctttggaatctacagggtggcccaaaaacctggaaatggaaaaatattacttattaattttaatgtatattaataagccattttaatgcttcatttccagtctcagtggccaccctgtatagctgggctattgagctcttgcgggaggagggagtggacagtctcccagccacacagactgatgttgcaccaaacattttttagcttccagacttccctggcccttagtgttacccttaactctccatttctctgcctttcacattctctactttttaaaaatctctgactccaccttcaccttatcattcttagcacatgaccatacttctgcttcccaaagaaaatgagcaattacttccttttccttttcctcctgtcatcaaatctgcagacatgtcatgcctaagtccagctttcctcctttctctgatctcagtctgcttcttccatttctgccctgaatcccgtcccctccccaacccccaaggacttcgctctatcagtcacctcttccctctcctgtatcttcaactcctcccattttactggcttcttcctcaagcctttccccaagcctttcccatctcaattacctcctcgcacatgcctctgcagaaaccaccccgtttcttccctcccctcggcagcctgttcttcctgttctgccctcatgatggcaccatcattgtgtcactaaaatcaatctctccgacatcatcaatggccttcctttgttgggaaacctaataaacactttatcttatttggtctttgttatgggttgaatgaggttaccccgaaatccatattagaagtcctaacccccagtacctcagaatgtgactttatttgggaatagggtcattgcagacgttattagttaggatgaggtcatactggaatgtgatgggctgcttatctaatatgactgatgtccttataacaaggagaaatttggagacagacacgcacatagggagaataccatgtgatgacaggagttatggagttggagtcaaaaagctatgggaacttaggagaaagacctggaacaaatcctttcctgcgcctagagagggagtatggccctgccactaccttgaattcaacgtttcggcttttcaaaactgtaagacaatacatttctgttgttcaaaccaattagtttgcagtactctgcgactgcagccctaacaaactaatacagtctcttggaggcatttggcaaggttgacaatggaagcactttcttacccctttaggtctgtcgcctttcttgttggggggtgttttctaacaattcctctccatctctctctctctagtttgtcttaaacattggtgttcttcagacttctgacctaggccttcttttcacttcacatattcccctgggtggtctcacccacttccagaaattacttaaattactgctcatgcagtactgtgctggaaactgtttaacaactggctctctgggaagaggggagactggttgatggtttttgctgatttctgtggtgtaaatactccctccatggccaattccaaactgccaacagtttaacaactggctcacaaattttctccaaatttaacatttggctttcacaggccaacaacgtggtacagccaactccagcacacctctgcttttgtgtcagagagaagtaacttatttttgtacaaaaggtaaaataaaaacacctgcaggccccctttttttccttaacaaactgctctagaaatagaatagctgaagcttcttttatgcattcatctgttatttccatgtcactgtggtggtgggattatttttcctttatttttcttgtatatggttgaaatactgtacctttgatcagttttagttttatggcatgttttgcacccatattaaatctagtttttgtcagagggcgtcaatattattttctcaaaacaagaaaatatttcattgcaaaggagacaaacaaaaaggtccttaataccaaaactttgaaatgtgatttcttgtacttggcagtgtccaagtggtaaacccaaacagtattgggttttcattttgttcaggaaagtctttgtctggcagcgacttacccttacatcaggcgggccttgctcattcattcacttaagtatttattaaacaccagcggtgtgccaagtacttatctaggtatcgggtagattctgataagtcagtcaggtccctgctctcagggagcttgcagcagagatgggggctgcaatagagagtaagccaaggaaatgaaaaaggaagttgatttcagagagtgatgaatgctatgaagaaaatgaaggcagcgcagtgtgatggagagtgacccaaggtggtacagtttgtacctctaaggaccagactgtgacccaggtcactcacagatgcccgtcatgtgatgccacagcaacttttccaggtgctcgtttcctcccacttcccagtctcttgcccagccgcgactgcttacaaatacagctagaggaatctaaatgaggttcctctatcatcaaacccaatcaaaatgccaaggaacagaatcagtgcctggctgaaggcagtggaacagggccagcctggagtggttctctctgaggaagttcctcatcttggttttagggccataccttgtgacctgtgagctaggggttgccagtccctgacatttctactgaggactcgcctgtctatattcccggcctgtatgtgtctcctgagttccagacacacagggcgaagcgcctgatggatggaagtatgttttttggtgttccattggtatctcaaattctacaaaacttagtgccccttctcctccctgttcctccccatcttcagtctatcacctgttcctcatccagcaaatgatattaccatcttccaaggagcttcccaggagtaatccttgactcctcctcaacatccaattaataatcaaatctaggccaggtacaatagctcacgcctataatcccagcactttgggaggctgaggcaggtggatcatttgaggccaggagttcaagaccagcctggccaacaaggtgaaacctgtctcatttaaaaaaagttattttaaaaactcaaatctattatttctacctctaagtgtgtcttgaatttatccatctctctccatctctgagctgttaccttacctcagtccatcacgttttgtctacgttaacatgaccagagtcttgttcttagtctggtgaggtcactccagctgcttcagatccttccatggctcaccgttgccctcatataaagttggcactcctggacatgtggcttacggggccctccgtgatgtggccctatttgcttctccattctgttctctcccagcctctctgcccccatctctaggcaccaaccacacccttctgctcgtcaatggtgccagcttctcttctatctctggtctttggacagacttttcccttcacctggaatgctttcttcaatcctaccccactctctttaatctagataaggtttattctttttgaatgtctagcagtgaaaccatttcccctgaaaaaccttctctaaccaaccccctaccctcagcccaaggtctagattaggagtccctctgaatgtttccatagcatttttaaagaattgcctatttacttgttcgtatctatcactaaactacaaattgtatgagaacagccactatctctgcctggttcaccattcatctccagcaactagcataatgcctggcagagtcagcctgcaacaaatatttgttgaataaattaacagatggctttatctccttaagtaaatcttgcttttttcacctattaaaacagacgcacaggccaggtgtggtggcccatgcctgtaatcccagcactttggcaggctgaggtgggcggatcacctgaggtcaggagttcaagaccagcctggccaacatggtgaaaccccatctctaataaaaatacaaaaattagctgggcatggtggtgggtgcgtatagtcccagctactagggaggctgaggcaagagaatcgcttgaacccaggaggcagaggtggcagtgagccgagatcatgccactgtactccagcctggatgacagagaccctgtctcaaaacacacacacacacacacacacacacacacacacacacacacacacacacaccaagttgtataatttaaaatataacgtgcttgttatggaacacttgtaaaatacaggaaagtaatgaaaaagtctaccatctagctcaccacataatgaccattgctatcatcctggcataattctctcctgtatataaatatatattcttttattgttaaaattacactatgagtactatttatttattttactgtggcaaaatgcgcaaaacataaaatcttgccattttaaggtatgcagtttggtgcattcaccacactcacattgttgtgcaaatatcaccactatctatctcagaacttcttcgtcttcccaaactgaaactctgtacccattaaacaatagtgcatcctctgttttcccctccctacaatttatttttatttgggtttgtaccaaactgaaaatagctgcttcttccttacttagttcagattagcatttccatttatttagccgtggttttgaggatgccatgacagatgccatccttcctagagctctttggggctgtcaggtatttcagtcagggtgaattcgggttgataacattttaaaatctcactttattctgaggttcctagtgtcagagcccaccgtatttttagggactcccaagttacaaacaaaaatatggtgaggaggaatcactgaagttttaacacaagagacttacattttgttcaatttctatcttttagtttatttcctaagcataaagaaatactttgaaaattttacatagcattatacatatttaattaagcatgagcacatcttaaaactttaaattttagatcagatctttaattcctaggatattaagaggtactggcaatttggccaggtgtggtggttcacgcctataatcccaacactttgggagggtgaagtgggcgaattgctagagcccaggaggtggaggctgcaatggcctgagatcacgccatcgtactccagcctggatgatgagaatgaaatcctgtctcaaaaaaaaaaaaaaaaaaaaaaagaagaagaagaagtattggcaatcagtgctccaggaataatttcctgacttgaaataaacctacatgtagacaaactaattaggccattccaagagttgctagcattggtttaatatgttttcagagcattccaggaagcagtgtggccagcattgcatgtttgatacttcagaaatgtatgacaggtgtttctcttacccaggtcttctgttttcttagttttgctcatgtaaatatttatgaacatcctcatctttttgagggaagggattatagatcattctaattccattttctagcatttggtaccattctaagcacatgataggcacccatttggagcatttttggcttgacagaatatgcatttagaattgttcaaattagaggtgtcagtgatgggaattagaatactatataattctaagtcatttgacttaaatacaaaagaatgattttccttggtggggaatggtgaagggaggcaggagttaagaagaggagaagagatcctaagtcatttataaacttctctggaaagacaggtgtgtgaagactttttaaaaagtcattcaccaaattgtgtgtgtgtgtgtgtgtgtgttttaaatagactttattttttagagcagttttaggttcacagcaaaattgaatgcaaggacagagatttcccataaaccccctgcccacacacatgcatagcctccctcattatcaacatccccaccagagaggtgtttgttctagttgatgaacctacactgacacatcattatcacccaaagtccatagttcacggcagggttcactgtcggtgtacattctatgggtttgagcaaatgtataatgacatgtatccaccattatagtaacatacagagtattttcagtgccctgcaaatcccctgttctccacctattcatccctccctctctgcatttccacccccagcccctggtaaccgctgatctttttactgtcccatagtttcggacgatctatttttcagacagacacagagctgtctttcccttagtttctattctatcatttctttctccccatccatcataaaaggctatgagttttttttaagtgttgaacaccatcctacttgtcaagttaaaacataagctcctggctgggtacagtggctcatgcctgtaatctcagcattttgggaggctgtggcagaagcatcacttgaagccagaagtttgagaccagcctgggcaacatagcaagaccccatccctccacacacaaacacacacacacacacacacacacacacacacacacacacacacacacaaaaacaagctcttgccagaattagagctacaaattgccctcaggttcctagaagatcagtccttcaattagattcagattgagatgcttcctcttttaaacaatgattccctttctatcatgcccaataagaaaacaaataaaaattaaacaatactgcctgtaatctcagctacccaggaggcagaagcagaactgcttcaacccggcaagcagaagttgcagtgaagtgagatcgcgccactgcactccagcctgggaaacagagcaagattctgtctcaaaaacaaaacaatgtgatttcctcctctaagtcctgcacagggaaatgttaagaaataggtccaccaggaaagaaggaagtaagaatgtttgactagattgtcttggaaaaaatagttatactttcttgcttgtcttcctaacagttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccagggtgaaaatttttaaagattcactctatattttaattaacgtcagtccgtcatgagaatgctttgagaaaactgttatttctcacacctaacaattaatgagattaacttcctctcccctcatctgacctgtggaggaatctgaacaagaggaggaggcagtgggcaggtttccttatcatgatgtttgtcatgttcagtgtgaggcctcacaaaaaaaaaaaaaaaaaaaaaaggcgtcctggatataactgagagctcattgtacagtaaatattaataaaacagtgattgtagctgaaggatagaactgcttggagggagcaagtgggtagaatcgcgtcaaactaaagagcatttctagccaaagacacaatgatagattgaaggatatttattctaaatatagaatatgggtgaacgagatctgtggacttctgggctccaacgttagattctgattttagcaagcttgtcaggggattctgatattgaaaggctgtggccttcacctgagaaacctgccctagggggccatgaaaatttgtcctgtctttcagaagtgctatcagacatcaaatggaagttaaatcgtatcttaacaattactaggatgggcgcagtgactcacacctgtaatcccaacactttgggaggctgaggcaggaggatcacttgagcccaggagttcgggaccagcctgggcaacatagagagacgttgtctctattttttaataatttaaagagaaaaaaatactgaaaatattgtatacaccactgaattataataatgtgtatataatgtatatattcattatgaggaatatttgattatttcatatattatatcttttccttctgtttattttatccagttatgaagtatttagaacaattcatcagtaattggggctaaattgacagaatagtaatcagagaaaatagaaaaagacagatgggttatctttgaataccaggttggagttgtttatgggtttgttttttgttttgggggcgtttttttagacagagtcccactctgttgcccaggctggagtgcagtggcacaagcatggcccactgcatccttgacctcttgggctcaagcaatcttcccaccttagcctcctgagtagctgggaccacaggtgcatgtcaccacacccagctaatttttttattttttgtagagacagtctttctatgttatccaggctgatctcaaactcctgcactcaagtgatccccctgccttggcgtcccaaagtattgggattataggcatagccaccacacccaacctagtttctatttagacttggccctttcccaccagtcatttgtgtccaaaagatctcataaatgtagacaggaaactgtcctttgctcatcagttttcttcatcctgtgtctagggggatggtcggtgggggaaactggggttatgcaagttcctctgaaacatcctctgtgagcccagggatggatgaggcaccagccgccagcgagtcagtgtgcagctttccagaaaggaagtcatcagccagtcagccggccctggcagccagcacccggcaaccctgctgtcttgtgataaagaaatggtctgcctgacaggatggtgtggatttttcttttttcttttttttttttttgagacagggtctggctctgtcgcccaggctggagtgcaatggcgggatcttggctcactgcagcctctgcctcccaggctcaaggcatcctcccacctcggtctcccgagtagctgggaccacaggcacacaccaccacgcccaactaagttttcgtatttttagtagaggcagggttttactatgttgtccaggctagtctcaaactcctgagctcaagctatccatctgccttggcctcccaaagagctggaattacaagcgtgagccactgtgcctgaccagggtggattttttcaagtgcacatgttgtggtcccagaagctctgatggtaccaaattccaagcgaaaaaaagtcaatggttcccacccatcctacctcccatgatggcaagaggaaatcaccacactgcagatacagtccatgtaaaacaaattgctatggattttgaaagtgaaccttaagagaactgcactatgttttcttcattagagttctctggtaatttccagctttttttttttttttttttagacagtgtctcgctttgtcgcccagtgtcacccaggctggagtgcagtgacgtgatctcggctcactgcaacctccgcctcgtgggttgaagtgattctcctgcctcagcctcctgagtagctgtattttagtagagacgaggtttcaccatttggccaggctggtctcgaactcctgacctcaagtgattcgcccatctcagcctcccaaagtgctgggattacaggtgtgagccactgcacccggccagtaatttcaagcttctgaggagccctttgaattgttaaataacttgtagctatgtccaacatatccatgttcagtgtatgttcgatatttcttaggaaacctgcccttggttgttttctttgtggtaattcatgagccggcaaatttgacatgtgttacagaatataccttttctctgctctcctacctcataaccagaacttaattatcctgctttagtcacataaatagctaactaaataaatatatgagatttcagtctgctcactgtgaaaatagaccttctaaatgatctcttccacttgcagatatttgcaaatatggatccatccctcctgatgtggaggagaagttacggttggaatggccctaccaggagcaattgctactccgagaacactaccagaaaaagttcaagaacagcacctactcaagtaagaaatgaaaggcaccctagagatgttccagccccaaagatatttgaataggttggactcgggcaccaatctagcaagtcctacggaagttgtataaagctgaaaatactgaagcatttcccaaatgggaaatcctaaactcaaaacttgctttttggtttttttgtttgtttgttttttcttcatctgacattgcttagtagtcacagaatgaaagataaatcaatcattcatgatctaacaatgaccttcagtgctctaaaaaactacggagtcaaggaaaacatgaatatattcctcatgtaaaattaaaatacagacatataaagggcaaaacatgaacatcattcataccttgaggtccgtccccctcccagaaataacccccagtatgccttggtttagagcattaagcaggagggccctgagtcactccagacagtcttgaccaccaagcagcattctctttttgtttcctctgtggcttttgcaaacacagggctagctcagctacccattagtatgttttcagtcactaaaacagtcttccagtcttcaaattaggatgacattgtcacatggggctttaaagcaagtgaaacaaggaaccccctttttttttttttttgagatggaatctcactcttgtcgcccagcctggagtgcaatggcgcaatcttggctcactgcaacctccacctcccaggttcaagagattctcctgccttagcctcctattcattatgaggaatatttgattattcagttcctgtagggtaaagatattacccccgatcatattattgattattgagtagctgagattacaggtgcctgccaccacgaccggctaattttttgtattttttagtagagacagggtttcaccatgttggccaggctccaggctcgtctcgaactcctgacctcaggtgatccacccacctcagcctcccaaagttctgggattacaggcgtgagccaccactcctggccacaatccttttttaactatgaaatatatttttatctgaagtttgatgtttatacccaactgagggatgatgttcccatatctcagttaaagaaataacctgctcagatacttcaagctcttcttttgacttttgaaaataaatgatcttgaagttactatactttgtttgggttagttaacattatttaaagtatattattttaattaattatctttgtaagattttactgtatactacctggagttcaatgtatcagatggatttcaaatttatgtacattttttatgtatatggtacagaaaaaaatgtgatccataagaaatcagaaaatagcgcatatgctaatagctaatgttgtcctctaaaaaacttatttttgcatttttaagagggggatatactctgacactttaataagtgtaattaattattgactggaatttggcatgaggcagggccatttcagatcccattaaaggaatgacacataccagagaaccacagaagtaaggccacatttgtaataaatcattatagctctgctaggagaagacccagttgtattaggtaattaatggatttgctcttaaaacacatgtcccggaagatataggtgagtcttggggggccgcattaaacattataccaatgtatcttacatttctaagaaagttttactactttacaggatctttctgttaccaaaatggaaggtttccaactccaggacttggctttcatagttcctacaccaggggaaatgccttcctttgctaactatgcaaccaggttagttagtgtaagtccagccaccctgttggcaatgctaaaaggtacaacaaacacagaattttatttgcatttgtaaacatttgatttctggctcgaaattttcagttttcatgggcacgtcatggaaacagaaatcttctgtgtttagtttgggcacctactcattgtagtgacaaatatttcagaagccaataggggattccacaaattgttctgaacctgtggctgagactggtaatggctgagtgacatggggacataccacaaaagaagaggtagcaaaaggctgctgagataaggacatgttcattgcttagctagtggcctgcacccttaaaacacatgtcccaggctgggtgctgtggctcacgcctgtaatcccagcactttgggaggctgaggcgggtggattacctgaggtcaggagttcgagaccaacctggccaacatagtgaaacctcatttctactaaaaatacaaaaattagccaggcatggtggcgggcgcctgtagtcccagctactcaggaggcaggcaggagaattacttgaatctgggaggcagaggttgtggtgagccgagattgcgccaccgcacgctagcctgggcgacaaagtgagactctgtctcaaaaaaacaaaaacaaaaaacaaacaaacaaaaaacaacaacaacaaaaaaacgggtatcccagaagatacaggtaagttttctaacacaggtcctcttgtatggtgcgttccacttaagtagaagatgacaaaaacatttgtcatgagaatatagactcacattttaaacctgtttgagcaggaaaaggaagcaatgttacagatgtaattctgggtgtgactgcagaaaggatgactcccttattaaagtagtcatcctgagtgagctaactctttgtacttcctcttctcctcctgttcccctcatcaccccattcttccgttgcctacacccaggcccacattggatgctgacatagacttacatggtacagtccaagggaaagatctgccatttttttcaatgtgtcatcttggttatcttcattccaaggatctctccactctttatacagtaagagatgagagtctggaaaggattgggaataagataatgaattgtaagttttaaattgttcttcgtattttggggaaggagtaggctaggtggtccttctgttttttttttgtttttttttttaaagtagatgtggccagacgtggtggctcacgcctgtaatcccagcactttgagaggctgaggcaggtggatcacttgatgtcaggagttcaagaccagcctggccaacacagtgaaaccccgtctttactaaaaatacaaaaactagccgggcttggtggcgtccacctgtagtcccagctactgcagaggtggaggcaggagaatcacttgaacccgggaggtggaggttgcagtgagccaagatcatgccattgtactccagcctgggcgacagaacaatactctgtctcaaaaaaaaagagaaaagaaaagaaaaaaagaatggatttgaactcagtcgtcaatagcctctattccaggagatgttacagttgattatgttatagggggtgtataatagaatttcgagctatgtaaattccaagtgcatttggaagaatgaagaaatggaggaagggtaaagtatgagtgcaagcattccaggttttttgaaaatgctataatctttgttcagggctagtacaaagtgctatttagctgtaagggttttttgtgatttacagacagttttcacatgtgtcatttcaaccttggttttatggcgaaggcatgtgatggtgcttgtcccaggactttagatccatatctgaggttcctgtcgggcaaagatattacccctgatcatattatagtctataagtgggagagttgtgcctggagctcaagtcttatgatttctgatccagggcacttcctacaacatgattttgcaatataaaagcctataatgtgtgactaaagcaggtcactcaccccttgtaacagactctagtaatggtactgccaccaaacggctgcgtgatattgggcaaagacttaccttatttgaatctcagtttcctcctagaaaaatgagggtggaggttaagcataggctgatgatcctaaagcctccatactgccctaaactgtggctctaagatccagtagaatgctgggtcacaggactctagggagcttttcaaacccaaatgtctgtcattccttgatggtaggcagcagtttatggaagtgggcgacacagcaaatatcaaaatacctaaagcagcttgcaagagttgtttctgcctagtggtctttatagttaatattaaatagttaattttttttttttttgagacagagtcttgctctgttacccaggctgcagtgcagtggcacaatctcggctcactgcaacctccacctcccgggtttgagcaattctgtctcagcctcccaagtagctgggactacaggtgcatgccactgcacccagctaatttttgtatttttagtagagacggggtttcaccatattgggcaggctggtctcgaactcttgacctcaggtgatccacctgcctcagcctcccaaagtgctgggattacaggcatgagccactgcacccagcttaaatagctaatatttaatattattctatagttattcaagtaattcaggccaaagacttagaaacaaaacaaaaagccacttttaaggagaaagggtgtaagtttgccagatagatagagatctttcttttttaactacaagagttcaggaatgaattactctttaacaaacgactatagatatacatgaaaattggaaggacttattatgcatatgataatcaatttaaagacaacacttaaaattatattgttgccactctcaaaaagtggtaatagaacagctaatggtttaaaaagcagagtacagaagttcccaaacttatggcaccttaatatcgcagaaaactttttaaagcatgcctaggccacaaaaaatacctgtattttgattattaaattgtaaggtctacacaacctaatagtaataggtccaatagtaatgctgtccaatagatgttgatgtttttttccttgcaaacttaaaagatcctacagtgcctctgtaaatagcactgcctggttagagttgaatttcagataaataatttttttcatgttaattatttttcttttctttacttttttttttgtttttttgtttttttgtttttttttttgagacagggtctcattctgttgcccaggctgctgtgcaatggcatgatcatggctcactgcagccttgacctccctgggctcaggtgatcctcccacctcagcctcccaagtagctagctgggactacaggtgcttaccatcatgcccggctaatttttgtgttttttgtagagatgtggttttgccatgttgcccaggctggtcttgaactcctgggctcaagtgatccgcccgcctcggcctcccaaagtgctaggatgacaggcatgagccactgcacctggcccctgggcgaagtatttcttaatggttacataggacatacactaaacattatttattgtctatatgaagttcaagtttaactaggtgccctgcacttttagttgctaaatcctgtagctgtacccatgcattcactggtgctccccagcttgccttgcacagagtttggaaaccatagtcctataactctaggccaattttttaatgtaaaatttgattcattttaaattaataaataataacaggaatttttttaaaaattgttttaaatataattaaaattatcaaaatattttttaactgaacttgtgactagagatatttagattatgaagagtggggtttatgctaactaatgacagtctggctatgcatgtggagcactgagctataaattgtggcttccccaattctcctgatgtcacttgaacaaaacctaagtgtcagaccagagcttctggtatcttccatgggatttcattcaacagctggagcaaatgaagtcagattgattttttttaatttgtccaattttgttgtctcaaaaacataattataatcatttattagaactagaatttcttcagtttaacaacagaaatagttattcattatgaaaagcgaatctggaggccttcattgtggtgccaatctaaccattaaattgtgacgtttttcttttaggaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatggtgagtaccccagggaacaattcattaataaggagattccccactagcattatttcttttcttttctttttcttttctttttttttttttttgagacagagtctcgcactgctgcccaggctggagtgcagtggcgccacctcggctcacttgaagctctgcctcccaaaacgccattctcctgcctcagcctcccgagtagctgggactacaggcacccgccaccgcgcccggctaatttttttttttttttttttttttttttttttgcatttttagtagagacggggtttcaccgtgttagccaggatggtcttgatctcctgacctcgtgatctgccctcctcggcctcccaaagtgctgggattacaggcgtgagccaccaggcccggctagcattatttcttatgacactttttttttttttttgagacggagtctcgctctgtcgcccaggctggagtgcagtggcgccatctcggctcactgcaagctccacctcccaggttcacgccattctcctgcctcagcctcccgagtagctgggactacacgcacccgccaccacgcccggctaatttttttgtatttttagtagagacggggtttcaccgtgttagccaggatggtctctatatcctgaccccatgatctgcccgcctcggcctcccaaagtggtgggattacaggcgtgagccactgcgcccggccaacactctttttattattagcaaatatacttctgcctgggcacattcttgcaagtgctcaacaatgcaacttttggaagtgcatgtggcagaaactcctgctgtatttattccagaacctattattgctaatcccagtttatgttacatttgaagtgagaaccagttggagccagcaacgttcccagctccaaagttcccttgagattttcagaatcacttaaccctattatgcttggcaacctggactcagcaaaactgggaagtcagcagtttgttttattcatcccttcctttctcagtttctcaaatgtgtcagttaatctcagtaaccccattgcaaccttcattacctgcccaagcggtctagaacttgccagtatagaatcctacgtgggtcaagctcctgactgtctccttcttcactctttttttgcaaagaacttgtaaattttaactataagtattcatgattcgccacatttattcaaaacatagagtgctttttccacatatcagccaatggaaataaggattaaatgggaaatgaaatgtagtaataggataagcacaagtcttcttcctgctcaaacttttttttttttttttttcagacaagatcttgctctgttacccaggctggagtgcagtggcgtgttcatagctcaatgtaacctccaactcctgggctcatgcaatctctcacacctcagccccctgattagctaggactacactatgcctagccaattttttttcttttgtctggttgtgttgcccaggctgtctcgatctcctggcctcaagtaatcctcctgcctcggccttctaaagtgctgggattataggcatgagccactgtgcccggtctcaaacctttttttccaaagtaaatgaagttattagatatggaatatagtctagttcccagatatccatatccattggtttattaccctcattattaacttcaaattgtttaatagaccctcatatctcagttatacagttaaaatttttgttttgtttttctggagtatcttatttataactatgagttttactttacttatttattttattttttgagacagacgcttgctctgtcactcaggctggagtgcggttgcgtgatcatggctcactatggcctcgaccttctgggctcaagtgatcctctccctcagcctcccaagctgagactacaggcatgcaccaccacatctagctaatttttttttttccccatggaacaaggctttactatgttacccagagtggtctcaaactcctggcctcaggggatcctcctgtctcagcctaccaaaatgctgggattacaggcatgagccatagcgccagacctggttttacttttcttgactttgaattacaagtttttgtaatttggaaaatgttttgttgcttttaaatactgctgtatgtttgcttttaaatacaacatttctcgatatatattttgagaattgctgtctttcagaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagaggtaggaactagaggatgcagaatcactttacttttcttctttttccttttgagacagagtctcactctgtcagccagactggagtgcagtggtacaatcatggctcactgcaacttcgacctcccaggctcaagcaatcctcccatctcagtcccacaaatagctgggactacaggtgcacatcaccacacctggctactttaaaaaaatttttttgtagagatggggtctccctgtgttgcccaggctggtctcttgaattcctgtgctcaagccatccttccacctcagcctcccagagtgccaggattacaggcatgagccaccacacccagccaccacttttcttaaaaaaaaaaaaagattctctctggtagacaatcctcaatagtccacatgttattaaacaatctgctgcctgaatacatgatttaccaaaaaaaggaaattttgacgggttcagaatatcaagggatctgaggcaaatgtcacctatgataaaatttgctatcaaaattaggaagtttgtgtttacctgatcctaaagcagtaaccagcccatttctagggaataaaactctcatgcgtatattgtgcatatatatgtattatatgactgagtgataataaaattttttttctagcttcctgaaggctggtggagaagtgattgattcagttacatggcatcagtaagtatgtctcctattcttaatactaggaaagtaaggctagctttatttattacctagtattcaaaaagttagttcatttaactgccaattgactgcagttcaaataagaaacaaatagtgtctcaagtagcactgtactccaattttaatattaataaaaaaaattttaagttattttaaataatgtagtggtttctataaagatcactttatacagaagaacagtgccaattaacccatggaacatataagtagctaaaaccaattgcttgccaaagaaccagtaacccaggagtacatgtccttgccactgtgttttttcaagacagagtaactgatttctagttacttgcatagaatggactcctcctcataactcccttccatcttggtctttccctagtagaacttctacctttttttagtaacaggtgagtgggagaggtaagaaggagaataaggtcagcaattaacctaaaagcagaaagtaaaatttgttattttttttctgaatattttctgtgtaatttagctactatttgaatggacggactgctaccagggaagattttctaaaccctgatgtattggacatttttatttcatctgtgcaaaaagttttccaggtaatagtctttttaaactttttaatgtaaaaccagaatccttattttatagtctagctagttctaaattctataggtatgtatatttacatgtttttctaattttagagaacaagcactatgacttatccactgttagttttccccttagcattgggtcttaccccatgtacgtgattagaaatttgaaatatttccaatagcctttagtagaattaactcacatagatgataagaatgggttggttcacttcatgttccttccacagcctactatttcaataaaagaaagtttcccaagacctaaatgactatgaacatattttataactatataggaggggtgggtctaggaatacaaagttttgaatgctgttaatcttcaacaccacagttgaaaccacaggtcagcttttttgcaattaccatggatacttttctgttctataggtggttgagagcaccaggcctggcaagaaggtctggttaggagaaacaagctctgcatatggaggcggagcgcccttgctatccgacacctttgcagctggctttatgtgagtgaagcagcgctggccttaggggtcagagtgcagctcttctccatccttctattctgctgaaatagctccccagccaaaaagcagatcaaagaccgtttcagtggctgagccccaaaattcatgccagattttgcaagaaaatgatttactaaagcttgagggacatctttaacaagtgttccaaattaatcactataaggatgaattgtttcagaaattttggcctttaattatggcccataaatatgtcaagtagtccttactctaaagaagtacactgtaaaagaatgcatatagccggatatggtagttccctgtaatcccaatactttgggaggccaaggtgggaggattgcttgagcccaggagtttgaggctgcagtgagttatgatggtgccactgcactctagactgggcaacagagtgagactgtctttttttttcccctctgtcacccagactggagggcagtggcacgatctcacctcactgcaacctctgcctcccggattgaagcgattctcctgcctcagcgtcctgagtagctgggactacaggagtatcaccgcactgggctaatttttgtatttttagtagagacggggttttgacatgttgcccaggctggtctgaaacccatgagctcaagtgatctgcctacctcagccttccaaaatgctgggattacggacatgagctaccacgcccggccacaccctgtctcttaaaaaaaaaaaaaatgcaagttagagcatattacagctttgtctctcaggaggatacttagtgtatgtagctataattcatagattcccaagaagtttagagcctaaagtatgaggtcccaccagaggggctatcattaaatttaaagatttgttaaatcatctcattgtccaacaccacaaacttgattgctttaaaatactggtttagttacatttagtaactctattagtgcttttaatctatactgctatatcctcacattgagattttttttcttttctcttccatcttcattcttttttctctcatcctcattcttataagcctagaatacatcacaaatcctttatgcccatggaagcaagaggaataaagaatggagatgtttgttttgccattaactaaagatctggggtgtcggggagaagggggatagagaaggagaagtgggaagaggtgtccataatagcttaggtgcaattctgcttattttacattttacccccgctgactgccactttttcttcagccctcacacattgtttgtgcagggacctcataggaccaggaattgtctatagaggtgggaatttgtctcaccctgaaagggatacctctagcatggtaatagtcttctaggatttgttatcatatggaaagatgtaaagggagggattctgctgctgctgctgctgctgcatgcagttgccatttcatttaaatgacttatttataattgatgacacttttctggcttcctgttaattcctccctcaaagatcaataaaccagaaccaggcatggtggcatgcacttgtggtcctgtaaccacccaacaggttcaccttgcctgctgtctagatagagccaattatcaagacaggggaattgcaaaggagaaagagtaatttatgcagagccagctgtgcaggagaccagagttttattattactcaaatcagtctccccgaacattcgaggatcagagcttttaaggataatttggccggtaggggcttaggaagtggagagtgctggttggtcaggttggagatggaatcacagggagtggaagtgaggttttcttgctgtcttctgttcctggatgggatggcagaactggttgggccagattaccggtctgggtggtctcaaatgatccacccagttcagggtctgcaagatatctcaagcactgatcttaggttttacaacagtgatgttatccccaggaacaatttggggaggttcagactcttggagccagaggctgcattatccctaaaccgtaatctctaatgttgtagctaatttgttagtcctgcaaaggtagacttgtccccaggcaagaagggggtcttttcagaaaagggctattatcatttttgtttcagagtcaaaccatgaactgaatttcttcccaaagttagttcagcctacacccaggaatgaagaaggacagcttaaaggttagaagcaagatggagtcaatgaggtctgatctctttcactgtcataatttcctcagttataatttttgcaaaggcggtttcagtcccagctacttgggaggctgagacaggaggattaatggagcccaggagtttgaggttgcagagagctatgatcacgccactgcactccagcctgggtgacagagtgagaccctgtctctaaataaataaataagtaaataaataaatacataaataaaatcaagatggtgtgcaattagaattgagcgattttgtttccaaacctcaagaaagcttggtcttgctctgtcccaggtggctggataaattgggcctgtcagcccgaatgggaatagaagtggtgatgaggcaagtattctttggagcaggaaactaccatttagtggatgaaaacttcgatcctttacctgtaagtgaccattattttcctaattctagtggagtagattaaagtcaactcaggacctctggtgttaacctcctatgaacagtcagtcctctcagtaactagccaaatcatgagatgatgaattagaaggagccttagatagcatccaatctaacatttttttgtgtgtttgaagagaagaaatcaagagctaggaataactttttaaaggtaagccatttgcagtatagtgtggattttgtttaaaaggggataatttgaaattttatgactcattatacaagacaaaataagttggattttcaaatgttttacaaagtaaatcaaagttataattgcctacagtacgcaaagcttcaaaacattttttatgttatgaaattgtaatttatttaaccttaaaatgagccagtaccatgtgtttgcttaaaaatctcatgctaagaatttactatgttgttaataatcttcaagatatttatgaataaagtcttatttctaatccttcctccaactgtatctggtgctaaatcaggaaatgtttcttcccaaaaagcctcgtggaagatctgtatgtctaaatatatgtcagggataatacagatgtagccctgcgaagcatgaccttgatttttatagtctaaaatgtcatttgcagatatctattttctaagaataattcctaaaagaattatttgaatgttgtaggaaagctaagaaattttgcaaagagcgtacgtgaaaatataagctaggcttttgtggtttgtggatagacttcccaacaaaattgctttttatctatagtgatccaagcttgtggaacatattagtcatctttttttagaaaattcttagaaaagtgatcttgcaaaaatggaatttatctttccccaagtatattctgtcatgtatagagttaaactaagcatagtaatttcaccagacaaacattcaaaatctactcctgacctttttatctcatccaaattttcccagggcccagacataaacctttgccttacgaactctttgtatatgcactaaatatgcttctccttcaaggttctcagtcagctagaaaaatgtgcaagagtaaatggtacccttctcacttgtagatccaagagaattagacttaaactcactctacatgtctgtgactttattttatttgcatgacagtcctgtgaggtggcaaggcaggtatcttggatccattttttagataaggaagttcaaattgagaagaggttgcatgatttacaggaagccatactgtagtcctatgttactcttaaaaatcccattcaaatcctgcttctgaggcctgcatactttctaccctaccagtcattgacccatgcttatgtctcctttgaaaacattgattccactcttgtctccagtgaaaaagtggaatttaagcagagaaacaaaagccatttgtcttgttaagtctactttccctctactttcaagaaggaaagttggggtatgtgttgaatggtgatttatttatttatttattattttaaaaattgatacaaggtcttactgtattgtgcaggctggtctcaaactcctgggctcaagtgatcatcccacctcagcctcccagtgttgggattacagcatgaaccattgtgcccaccaccgatccgcagttttttaagaaaaacttttactatagaaaattttaatcatatacaaaatacagaggaaagtatatgaacccactttaggagactagaatatgccaccccaaaatatgccactttggcataaggattatttcgagctaaaggcaactgggaagaaacacatagaagaaaagttctctgtccttctccatttgcctaaaagcaggacatgaatcttaaaagtccccctccttccctttctaccaggaaaaacaagagttaatcactgaagataacttcagacccttatcagtgtagagatggcactagaagaatctatattacatactcatttattttccttcccacaacttgccaccccagagactaaaaatccttttcctttgtcatgtctcttgtccaaaaatttgctctataagctggagttctaagccacctctttgagaattacttgttccctggtattttctgttaacatacatgtattaatatacatgttaacaagcttctgtttgtttttctcctgttttctgtcttgttacagaggtccatcccaactaagaactaaagagtaggaggaaaatataatttcctcctgcatactttgatcttgtttaatccgtaacccttcccacttttcacctcctacctattagattactttgaagcaaatttcagatatattactttatctataaatatttcagtatgtgctaggtgtggtggctcacacctgtaatcccaacactttgggaagctgaggcaggaggatcacttgagcccaggagttcaagaccagctacggcaacaaaaaatcaaaaacttatctgggcatggtggcacatgcctgtggtcccagctacatgagaggctgaggcaggaggatcgctttagcccaggaggttgaggctgcagtaagctgcattcacaccactgcactccagcctgggtgacagagtaagaccatgtctcaaaaaaatacatattttagtatgtatcctttttgtaaaaacacaatacttttatcatactttaaataataacaataattccttagtatcaccaaatattttgtcagtgtctcacattttccttattgtctaaaatattgttgatagttattcaaatcagaatccaaacaaggtccatatattacatttggttgacaagtctcttaagtttgttcatctttaagttcttcctccctctctttcatctcttgtaatttattaatgtgaaaaaacaggtaatttgttctatagtatttcctacattatagagtttgctacatttattccctatgatatcatttagcatgttcctctgtcccctgtgtttcctgtaaactggtagttatacctagaagcttgagtttattcaggtttttaattgtattttttttgcaagaattctttattatctgcttctggaagcacagaatgtctggttgtgtctggttttgatcttgacagctactgatgaccattgcctaatccattactttattggggtggggggaataaggttttaaaataaattttttttaaagatttttttaactgttattttgagacagtgtctcatttcgtttcccaggctggagtgcagtggcacaatcacggctcactgcagccttgacctcctgggatcaggtgatcttctcacctcagcctcctgggtacctggaactacaggtgcacaccaccacacctggctaattttttgtattttgtgtacagaaggggtttcatcatgtttcccagactggtcttgaactcctgggttcaagtgatctacccacttcagcttcccaaaatcctgggattacactttggccaccgtgcctggcctaaatgaaattatttgtctctaaacagacagaagttttactttaaaaatttgtctttgtgtgtacatgtgtttgtgtatgtgtgtgtgtctaaaagtttggctttgagctttgctttgaattcttggatgaacaataaccaagaatacttaaactctgatcattcttgacagatatcccctacaggctatggccttttgaattgtgtcctccagtgataaaaagcagcaagcacgatactgctctcagattcatggtggtcacatgtgaggtgaaaaaaaaaaaaaagatgaatcctatttaaatgcccccaggataacagtgatactctttgtaggataactatttgcttgccactggtttcattaaataaggacataagtaaagatctatttttgtctctttctccccaaccaccacaactaggattattggctatctcttctgttcaagaaattggtgggcaccaaggtgttaatggcaagcgtgcaaggttcaaagagaaggaagcttcgagtataccttcattgcacaaacactgacaagtaagtatgaaacacaccctttaccaatcatcaagttttagtgggtaagcctgtaactttactcaaacaccctgttgcatgtgtctatacattgcataagtataggcagttgcaatttagtaaagttttatacaacgattttattttattttatttttagaagaaaaatgctacttttgttgttgttgttttttgagacggggcctcgctcgtcacccaggctggagtgcagtggtgcaatctcagctcactgcaacctccgcctcccgggttcaagtgattcttgaagaggagaacaataataacaacaatattattttcaaaagttgtgaccgcagtttctggagttgagaagacatcgagatttttgtagcctcatactcttgctttaggtagcaaaaaatgttcctaaatctcaggaatattctctagataggtttcaatctatcattcctgataagatgatgctgaaatactaattctagccaaaaaagaccagctaccatttccgattgttggggactgggaactctggatagtgaggaccccagtaggaagtagcgaggggaatggtttgaatggataaattcataaaaaatgtcagtagatttaattttcttatacatttcagtctttttataaggctaggaaaagcccctgtttttatggtttataatttgaattcacatgaacccacaaaatttgccttttaccttcctatgtctgaaaatggatagtctggctggcctcttaacaacccagctggcagagctgtgaggatctcagtgtgctctagcccagacattggtagcatgaacggcaacatttttaattgtgttttcaaaataggagcacactagcggtctaaaacgatcataaaagaaggatactaagagggcccactgtcattatggatcctaatacttaggatgcattatggattgtcattatggatactaatacttaggatcacatttgtaattgagtttttaattgcttaaattagatacatatttctattaagttaacctctttgcttttagtccaaggtataaagaaggagatttaactctgtatgccataaacctccataatgtcaccaagtacttgcggttaccctatcctttttctaacaagcaagtggataaataccttctaagacctttgggacctcatggattactttccaagtaagtaattttccttgttcattccaaactttcaataaatttattggtgtttatcagaatagagagtttggacagggagcaaaagacaaagtcaactatatcaagttctaataattcttaatattcaggaaatttatgtatgaatacttactaatatgagtataactcatcctaagagtctaaagcaaaaggatgtgaacacaaactagcagttatcttagagaataagtttgcatttcaaaataacttgacatatcaagatccactcaacgcatttaaattatttactctaaaaagacataattcttggtaacacattcactaaagcaaaatatacctttatataattgctatcaaaggtatgtgggttggtataaaatatcataccatgtgagatcagtgtgattcctttacagcattaatttttattggttagagtaagaaaaagaatagctagagtatatttcttaagtagattctcatacactttggtttcaaaaaccaattattgactacatcttataaaagcctgtattcaatggagtgccaaaaaatgactatgagtcttaaagagttaggcatataaatattttaaggtttctgttcaatgtatgttggaaggagttcctttctcatgactattctcatattggagcataaaaagagtttacaggcttggcgcagtggctcatgcctgtaatcccaatactttgggaagctgaagcaggcagatcacttcagcccaggagtttgagaccagcctgggcaatatggcaaaactctctctacaaaatataccaaaattagccaggcgtggtggtgcatgcctgtagtcccagctacttgggaagctgaggtgggaggattgcttgagcccaggggggtcatggctgcagtgagctgtgatggtgcctctgtcacccagcctgggtgacagagtgagaccctgtctcaaaaaaataaataaataaaaattaagagtttacaaaattctcaccatctcctcccatctttgcaaatgccacataagtgatgtgttccaggactattagcctcggaacctgaggcagtacagtaagcacgctttctccaaagtcctgtcccccacagacaaacattatttacactgggtactgctcttttattttttcccctctatgctttattttactataactataatcatataacatgtaataggaaaaaggcagggtcgggggagagatccagaagtcttcccaagagcctttccaacatagcctctgtagacattttttctttcttctttttttttttttttttttttctgagacagagtctcactctgttgtccaggctagagtgcagtggcgtgatctaggctcactgcaacctccgcctcctgggttcaagcaattctcccacctcagcctccctagtagctgggattagaggcatgcatcaccacgcctggctaatttttgtatttttagtagagatgaggtttcaccatgtgggccaggctggtcttgaactcctgacctcaagtgatccacctgccttagcctcccaaagtgctaggattacacgagtgagccaccgtgccctgcccctattacattctgatcacacatttcatgttttataattggaaaactggtgaaattatagacaatgttttgttcccctaaattctctttgatgagtatatattacttacactcttctgtctttaaaattttgcaaaatagtatcctagataagtttatgagtgcacagtctgtacgcttactcatattaatgacctcggagagttaaacaacagtcacctttaaaaattattactatcattatcattatttttgaggcgggggtctcattctgtctcccaggctggagagtagtggtgcggtcacagctcactgcagccaccgctacctgggctcaagtgatccttcctcctcagccttctgagtagctgagaccacaggcttatgctaccacacctggctaattttttaactttttgtagagacgatgtctcattatgttgcccaggctggtctcaaactcctaagctcaagtgatcttcctcagcctcccaaagtgctgggattacaggcatgaaaaactgcacccagccctaaaaattattagggtcctgcatagtaagactttaataaatatttaaatgaacatctggtttttttaaaaaaaaaatagagacaaggtctcactatattgcccaagctggtctcgaactcctggactcacgcaatcctgctgccttagccgcccaaagtgctgggattacaggcatgacccacctcatctgggctgagtgaacatatttttaacataaaggccgtattttatatttatctcatacattttgcccagcatccccatttccgccgaatctgttgcttgctaattccttccagcttcatttcatctgaaatttgacaaacatcttctatttctttgtcgtcatgttattgacttcagaatataaaataaaacactatacccaaattaaaccccaccctcattgcccagcctgatgtgaaaataatcagcatacattaagcttacccttgatatatgtgtagcatcttttagataaatatacagctgattaagcaatatagcctgatggtataatatcttgcccatgtacctcatcttatctccagcaggattaattcacagtgatcagatttacctttaaactttgtagcaaaatatcctctccaaaagcatatctaaaacttttgtgtgtactcttgcaagtttcttaatttcatgcagaacaggctcttaccactgttagctggagatattttcaagacctatttttgtttgtggtttcctgatgatggtcatggcatttcccccttcactccatctaaaaattgaggtgatacaggcttttaaacaaaaccaactcatatagactgagtacaactgcaatgcaggcatgctaacctctgctacaatcatgggcgtgctattgatatgtcttaagttacagaacacagggctgagcgtctcattaggtcaaaatgtaaaccagtttttctgctcactgatgcttaatgaggacagggtgtgagagatttctttaaggaaaacaaatatataataatgctacatggaaaaatatctaacattagagaattaagtaaataaactaatatactcacaccatggaatcttgtgcagacattaaaattatgtagtggatggatgtttaatggtgtgagaaaaagttaggatgtgctggggtggggggaagaatcaagttttaagaaaatacagtatacccatacttaagtaaaaaaaaaaaaaaaggtatgtacagtcatgtgttgcttaatgatggggatacattccgagaaatgtgtcgataggtgatttcatccttgtgtgaacatcatagagtgaacttacacaaacctagatggtctagcctactatgtatctaggctatatgactagcctgttgctcctaggctacaaacctgtaaagcatgttactgtagcgaatatacaaatacttaacacaatggcaagctatcattgtgttaagtagttgtgtatctaaacatatctaaaacatagaaaactaatgtgttgtgctacaatgttacaatgactatgacattgctaggcaataggaattataattttatccttttatggaaccacacttatatatgcggtccatggtggaccaaaacatccttatgtggcatatgactgtatacatgtacacaaaaaatagatgaaagaatgaatatacatcaaaatatttaaaatggttataatgacttaggttacttttatttatcttagtaataataatgatgatagataatacttttatagtgtttactatataaaagacactgttataagtgttctacatactttacatgtattacctaaatgatataaatataactctgacagtaactaatcttatacgttctcttttcttttttttttttttctttttttagacagaatcttgctctaccaggctggagtgcagggtgcaatctcggctcactgcaacctccgcctcccaggttcaaacgattctcatgtctcagcctcctgagtagctgggactacaggcacacaccaccatgcccggctaatttttgtatttttgggtagagatggagttttgccatgttggccaggctgatcttgaactcctggcctcaagtgatctgcctgcctcagcctcccaaagtgctgggattacaggtgtgaaccactgtgctcggcctaatcttacaagttttcaatatttaaagagtgctaactttgttgacaatataaaacatatttgagaaaaagagatataagcatcttatttagaattatgaaaatatcaatagacctacagccgactaaagcttttcttcataagctcttgcctatattgattcgctcctgtgaatatgcattaatttgatttaaataataagtatgtataagaaataacacttttccttaatttttaagaacgttcaacagtttttaatttgaattccaatagtgaaatacatagaaaatataaaattttctgtagtttagccaaattgtttttgtttcaccacagcattctaccaaaatttcttaataacagtaagaaaatgaatgcatacctcctgcagggagaggggagttaggcagtttatgggcatagttacaagtgagaaatttcattggctaccatttacgctaaattcataaaaactgcattcaattctatatatctattttctttacataaaaaaggtttcaattattggccattaaataaaatagccaccattccagaagttgtgtcatgtttatcctttttataccaccatcatattgcctattatatagattgtgtgtgttccattttctgtaatgggccagacagtaagtatttctggctttggagtccatatggtctctatcataactactcatctctgccattgtagcttaaagattatctaggtcaaatgcctaagtgatatagtgttgaaatacaagttatataatataggctgccacaaaaaaaaatttatttggtctaaaaaagatttcatgacttttgtagcagcatgggtggggcatgcaccacttggttaactcggtgtatctttctcctttgcagatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactgaatttttcaagtatactaagagtaaagcaactcaagttataggaaaggaagcagataccttgcaaagcaactagtgggtgcttgagagacactgggacactgtcagtgctagatttagcacagtattttgatctcgctaggtagaacactgctaataataatagctaataataccttgttccaaatactgcttagcattttgcatgttttacttttatctaaagttttgttttgttttattatttatttatttatttattttgagacagaatctctctctgtcacccaggctggagtgccatggtgcgatcttggctcactgcaactttaagcaattctcctgcctcagcttcctgagtagctgggattataggcgtgtgccaccacgcccagctactttctatattttttgtagagatggagtttcgccatattggccaagctggtctcgaactcctgtcctcgaactcctgtcctcaagtgatccacccgcctcagcctctcaaagtgctgggattacaggtgtgagccaccacacccagcagtgttttatttttgagacagggtatcattctgttgcccaggcttgagtgcagtggtgcaatcatagatcactgcagccttttaactcctgggctcaagtcatcctcctgcttagcctcccaagtagctaggaccacagacacatgccatcacacttggctatttttaaaaaattttttgtagagatggggtctcgctatgttacccaaactggtcctgaactcctggactcaattgatcctcccaccttggccttccaggtgctgggatttctttgggagtacagcatggtacagcaggagatcatttgatgttacctctgtgcagtgttgctagtcagcgaaagactataatacctgtggggacagcgattagccaccacaaccagtctttatttaaagttattaaaaatggctgggcgcagtggctcacacctgtaatcctagcactttgggaggccgaggcagatggatcacctgacgtgaggaatttgagaccagcctggccaacatggtgaaaccccatctctactaaaaaatacaaaaattagctgggtgtggtcctgtagtcccagctacttgggaggctggggcaggagaattacttgaacccaggaggcagaggttgcagtgagccgagattgtgccactgcactccagcctgggtgacagagagagattccatctcaaaaaaacaagttattaaaaatgtatatgaatgctcctaatatggtcaggaagcaaggaagcgaaggatatattatgagttttaagaaggtgcttagctgtatatttatctttcaaaatgtattagaagattttagaattctttccttcatgtgccatctctacaggcacccatcagaaaaagcatactgccgttaccgtgaaactggttgtaaaagagaaactatctatttgcaccttaaaagacagctagattttgctgattttcttctttcggttttctttgtcagcaataatatgtgagaggacagattgttagatatgatagtataaaaaatggttaatgacaattcagaggcgaggagattctgtaaacttaaaattactataaatgaaattgatttgtcaagaggataaattttagaaaacacccaataccttataactgtctgttaatgcttgctttttctctacctttcttccttgtttcagttgggaagcttttggctgcaagtaacagaaactcctaattcaaatggcttaagcaataaggaaatgtatattcccacataactagacgttcaaacaggccaggctccagcacttcagtacgtcaccagggatctgggttcttcccagctctctgctctgccatctttagcgctggcttcattctcagactctggtagcatgatggctgtagctgtttcatgggccccttcaaacctcatagcaaccagaggaagaaaatgagccattttttgagtctccttcatagacttgaataactctttttcagagcttctcacagcaaacctctcctcatgtctcctcatgtcttattgttcagaaatgggtaatgtggccatttcaccagtcactgccaacaacaacgaggttcctataattgtctctgagtaaccctttggaatggagagggtgttggtcagtctacaaactgaacactgcagttctgcgctttttaccagtgaaaaaatgtaattattttcccctcttaaggattaatattcttcaaatgtatgcctgttatggatatagtatctttaaaattttttattttaatagctttaggggtacacactttttgcttacaggggtgaattgtgtagtggtgaagactcggcttttaatgtacttgtcacctgagtgatgtacattgtacccaataggtaatttttcatccattaccctccttccgccctcttcccttctgagtctccaacatcccttataccactgtgtatgttcttgtgtacctacagctaagcttccacttataagtgagaacatgcagtatttggttttccattcctgagttacttcccttaggataacagcccccagttccgtccaagttgctgcaaaatacattattcttctttatggctgagtaatagtccatggtacatatataccacattttctttatccacttatcagttgatggacacttaggttaattccattcaatttcattcaatttaagtatatttgtaaggagctaaagctgaaaattaaattttagatctttcaatactcttaaattttatatgtaagtggtttttatattttcacatttgaaataaagtaatttttataaccttgatattgtatgactattcttttagtaatgtaaagcctacagactcctacatttggaaccactagtgtgttgtttcaccccttgttatactatcaggatcctcga

SEQ ID NO: 43 - DNA, 2396 bp, Mus musculus
tttctagttgcttttagccaatgtcggatcaggtttttcaagcgacaaagagatactgagatcctgggcagaggacatcctagctcggtcagatttgggcaggctcaagtgaccagtgtcttaaggcagaagggagtcggggtagggtctggctgaaccctcaaccggggcttttaactcagggtctagtcctggcgccaaatggatgggacctagaaaaggtgacagagtgcgcaggacaccaggaagctggtcccacccctgcgcggctcccgggcgctccctccccaggcctccgaggatcttggattctggccacctccgcaccctttggatgggtgtggatgatttcaaaagtggacgtgaccgcggcggaggggaaagccagcacggaaatgaaagagagcgaggaggggagggcggggaggggagggcgctagggagggactcccgggaggggtgggagggatggagcgctgtgggagggtactgagtcctggcgccagaggcgaagcaggaccggttgcagggggcttgagccagcgcgccggctgccccagctctcccggcagcgggcggtccagccaggtgggatgctgaggctgctgctgctgtggctctgggggccgctcggtgccctggcccagggcgcccccgcggggaccgcgccgaccgacgacgtggtagacttggagttttacaccaagcggccgctccgaagcgtgagtccctcgttcctgtccatcaccatcgacgccagcctggccaccgacccgcgcttcctcaccttcctgggctctccaaggctccgtgctctggctagaggcttatctcctgcatacttgagatttggcggcacaaagactgacttccttatttttgatccggacaaggaaccgacttccgaagaaagaagttactggaaatctcaagtcaaccatgatatttgcaggtctgagccggtctctgctgcggtgttgaggaaactccaggtggaatggcccttccaggagctgttgctgctccgagagcagtaccaaaaggagttcaagaacagcacctactcaagaagctcagtggacatgctctacagttttgccaagtgctcggggttagacctgatctttggtctaaatgcgttactacgaaccccagacttacggtggaacagctccaacgcccagcttctccttgactactgctcttccaagggttataacatctcctgggaactgggcaatgagcccaacagtttctggaagaaagctcacattctcatcgatgggttgcagttaggagaagactttgtggagttgcataaacttctacaaaggtcagctttccaaaatgcaaaactctatggtcctgacatcggtcagcctcgagggaagacagttaaactgctgaggagtttcctgaaggctggcggagaagtgatcgactctcttacatggcatcactattacttgaatggacgcatcgctaccaaagaagattttctgagctctgatgcgctggacacttttattctctctgtgcaaaaaattctgaaggtcactaaagagatcacacctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 44 - protein, 535 aa, Mus musculus
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDALDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI

SEQ ID NO: 45 - DNA, 2396 bp, Mus musculus; identical to SEQ ID NO: 43
  CDS (594)..(2198)

SEQ ID NO: 46 - DNA, 385 bp, Rattus norvegicus
cggccgctgctgctgctgtggctctgggggcggctccgtgccctgacccaaggcactccggcggggaccgcgccgaccaaagacgtggtggacttggagttttacaccaagaggctattccaaagcgtgagtccctcgttcctgtccatcaccatcgacgccagtctggccaccgaccctcggttcctcaccttcctgagctctccacggcttcgagccctgtctagaggcttatctcctgcgtacttgagatttggcggcaccaagactgacttccttatttttgatcccaacaacgaacccacctctgaagaaagaagttactggcaatctcaagacaacaatgatatttgcgggtctgaccgggtctccgctgacgtgttga

SEQ ID NO: 47 - DNA, 541 bp, Rattus norvegicus
  misc_feature (507)..(507): Any nucleotide
aaatcaggacatatccttcacttatttgcctcttggtcatattggaggcatttgtattcatttttaataaccctcaaaatagtgcatgcaaagtgctaagcgtcatttgccacatggtgccattaactgtcaccacctgcagtggtctacttagagaacaccgcactggatgttaacactgaagcgcgtgccccgccctcccgaggctctggatccagcgttgaagcttgccccgccctcccgaggctctggatccagcactggagcatgccccgccctcccgaggctctggagcttgctaaggagtccgctccctaccgctggggttttgctttattcttatgaatgacacccctgaccgctttcgtctcaggggtactgtaatgccttttattttcatatacaagctgcgattttggcatttcttatgacaaaaaacccataggaaaaggcgggcacgcttagtgagcttcctgcggggagaggtttttctgttagagctggcanggtctgctcatcgaccatcttcaggcctcgtgcc

SEQ ID NO: 48 - DNA, 20 bp, Artificial sequence
  Chimeric antisense oligonucleotides containing, 2′-O-methyl RNA“wings” (5 nucleotides on each end) with a phosphorothioate DNA center (10 nucleotides)
gaagaagtccaggtccacga

SEQ ID NO: 49 - DNA, 20 bp, Artificial sequence
  Chimeric antisense oligonucleotides containing, 2′-O-methyl RNA “wings” (5 nucleotides on each end) with a phosphorothioate DNA center (10 nucleotides)
gcaggagacaagcctcuggc

SEQ ID NO: 50 - DNA, 20 bp, Artificial sequence
  Chimeric antisense oligonucleotides containing, 2′-O-methyl RNA “wings” (5 nucleotides on each end) with a phosphorothioate DNA center (10 nucleotides)
guagugttctcggaguagca

SEQ ID NO: 51 - DNA, 20 bp, Artificial sequence
  Chimeric antisense oligonucleotides containing, 2′-O-methyl RNA “wings” (5 nucleotides on each end) with a phosphorothioate DNA center (10 nucleotides)
gaagagcagtagtccaggag

SEQ ID NO: 52 - DNA, 20 bp, Artificial sequence
  Chimeric antisense oligonucleotides containing, 2′-O-methyl RNA “wings” (5 nucleotides on each end) with a phosphorothioate DNA center (10 nucleotides)
gaaggtggactttctuagaa

SEQ ID NO: 53 - DNA, 20 bp, Artificial sequence
  Chimeric antisense oligonucleotides containing, 2′-O-methyl RNA “wings” (5 nucleotides on each end) with a phosphorothioate DNA center (10 nucleotides)
cuucuccaccagcctucagg

SEQ ID NO: 54 - protein, 15 aa, Artificial sequence
  Synthetic peptide
RPGKKVWLGETSSAY